(19) United States
(12) Patent Application Publication
(10) Pub. No.: US 2011/0257170 A1
(43) Pub. Date: Oct. 20, 2011

(54) 4-MORPHOLINO-PYRIDO[3,2-D]PYRIMIDINES

(75) Inventors: Vincent Pomel, Groisy (FR); Pascale Gaillard, Collonges-sous-Saleve (FR); Gwenaelle Desforges, Annemasse (FR); Anna Quattropani, Geneva (CH); Cyril Montagne, Saint-Genis-Pouilly (FR)

(73) Assignee: MERCK SERONO SA, COINSINS, VAUD (CH)

(21) Appl. No.: 13/122,799

(22) PCT Filed: Sep. 30, 2009
(86) PCT No.: PCT/EP2009/062664

§ 371 Date: Apr. 6, 2011

Related U.S. Application Data
(60) Provisional application No. 61/195,408, filed on Oct. 7, 2008.

(30) Foreign Application Priority Data
Oct. 3, 2008 (EP) 08165839.5

(51) Int. Cl. A61K 31/5377; A61P 35/00; A61P 25/28; A61P 37/06; A61P 29/00; A61P 1/00; A61P 17/06; A61P 27/02; A61P 17/00; A61P 35/02; A61P 9/10; A61P 35/04; A61P 1/16; A61P 25/16; C07D 471/04

(52) U.S. Cl. 514/232.5; 544/117; 544/80; 514/234.2

(57) ABSTRACT

This invention relates to compounds of Formula (I) as Pi3k inhibitors for treating autoimmune diseases, inflammatory disorders, multiple sclerosis and other diseases like cancers.

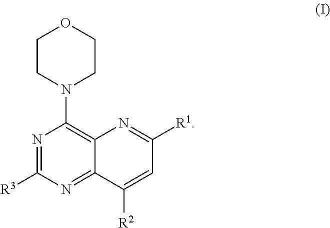

The invention relates to compounds of formula (I) and related formulae, their use as medicament and their use for treating autoimmune diseases, inflammatory disorders, multiple sclerosis and other diseases like cancers.

### BACKGROUND OF THE INVENTION

Phosphoinositide 3-kinases (PI3Ks) have a critical signalling role in cell proliferation, cell survival, vascularization, membrane trafficking, glucose transport, neurite outgrowth, membrane ruffling, superoxide production, actin reorganization and chemotaxis (Cantley, 2000, Science, 296, 1655-1657).

The term PI3K is given to a family of lipid kinases which, in mammals, consists in eight identified PI3Ks that are divided into three sub-families according to their structure and their substrate specificity.

Class I group of PI3Ks consists in two sub-groups, Class IA and Class IB.

Class IA are a family of heterodimeric lipid kinases consisting in a 85 kDa regulatory unit (responsible for protein-protein interactions via the interaction of Src homology 2 (SH2) domain with phosphotyrosine residues of other proteins) and a catalytic sub-unit of 110 kDa that generate second messenger signals downstream of tyrosine kinases, thereby controlling cell metabolism, growth, proliferation, differentiation, motility and survival. Three catalytic forms (p110α, p110β and p110δ) and five regulatory isoforms (p85α, p85β, p55γ, p55α and p50α) exist for this class.

Class IB are stimulated by G protein bg sub-units of heterodimeric G proteins. The only characterized member of Class IB is PI3Kγ (p110γ catalytic sub-unit complex with a 101-kDa regulatory protein, p101).

Class 1A PI3Ks comprises α, β and γ isoforms, which are approximately of 170 kDa and characterized by the presence of a C-terminal C2 domain.

Class III PI3Ks includes the phosphatidylinositol specific 3-kinases.

The evolutionary conserved isoforms p110α and β are ubiquitously expressed, while δ and γ are more specifically expressed in the haematopoetic cell system, smooth muscle cells, myocytes and endothelial cells (Vanhaesebroeck et al., 2001, Annu. Rev. Biochem., 70, 535-602). Their expression might also be regulated in an inducible manner depending on the cellular-, tissue type and stimuli as well as disease context.

PI3Ks are enzymes involved in phospholipid signalling and are activated in response to a variety of extra-cellular signals such as growth factors, mitogens, integrins (cell-cell interactions) hormones, cytokines, viruses and neurotransmitters and also by intra-cellular cross regulation by other signalling molecules (cross-talk, where the original signal can activate some parallel pathways that in a second step transmit signals to PI3Ks by intra-cellular signalling events), such as small GTPases, kinases or phosphatases for example. Phosphatidylinositol (Ptdlns) is the basic building block for the intracellular inositol lipids in eukaryotic cells, consisting of D-myo-inositol-1-phosphate (Ins1P) linked via its phosphate group to diacylglycerol. The inositol head group of Ptdlns has five free hydroxy groups and three of these are found to be phosphorylated in cells in different combinations. Ptdlns and its phosphorylated derivatives are collectively referred as inositol phospholipids or phosphoinositides (PIs). Eight PI species have been documented in eukaryotic cells (Vanhaesebroeck et al., 2001, above). PIs all reside in membranes and are substrates for kinases, phosphatases and lipases.

In vitro, PI3Ks phosphorylate the 3-hydroxyl group of the inositol ring in three different substrates: phosphatidylinositol (Ptdlns), phosphatidylinositol-4-phosphate (Pl(4)P) and phosphatidylinositol-4,5-biphosphate (Pl(4,5)P2), respectively generating three lipid products, namely phosphatidylinositol 3-monophosphate (Pl(3)P), phosphatidylinositol 3,4-bisphosphate (Pl(3,4)P2) and phosphatidylinositol 3,4,5-trisphosphate (Pl(3,4,5)P3.

The preferred substrate for Class I PI3Ks is Pl(4,5)P2. Class II PIKs have a strong preference for Ptdlns as substrate over Pl(4)P and Pl(4,5)P2. Class III PI3Ks can only use Ptdlns as substrate in vivo and are likely to be responsible for the generation of most Pl(3)P in cells (Vanhaesebroeck et al., 2001, above).

The phosphoinositides intracellular signalling pathway begins with the binding of a signalling molecule (extracellular ligands, stimuli, receptor dimidiation, transactivation by heterologous receptor (e.g. receptor tyrosine kinase)) to a G-protein linked transmembrane receptor integrated into the plasma membrane resulting in the activation of PI3Ks.

Once activated, PI3Ks convert the membrane phospholipid Pl(4,5)P2 into Pl(3,4,5)P3 which in turn can be further converted into another 3′ phosphorylated form of phosphoinositides by 5′-specific phosphoinositide phosphatases, thus PI3K enzymatic activity results either directly or indirectly in the generation of two 3′-phosphoinositide sub-types that function as second messengers in intra-cellular signal transduction (Toker et al., 2002, Cell Mol. Life. Sci. 59(5) 761-79).

The role as second messengers of phosphorylated products of Ptdlns act is involved in a variety of signal transduction pathways, including those essential to cell proliferation, cell differentiation, cell growth, cell size, cell survival, apoptosis, adhesion, cell motility, cell migration, chemotaxis, invasion, cytoskeletal rearrangement, cell shape changes, vesicle trafficking and metabolic pathway (Stein, 2000, Mol. Med. Today 6(9) 347-57). Chemotaxis—the directed movement of cells toward a concentration gradient of chemical attractants, also called chemokines is involved in many important diseases such as inflammation/auto-immunity, neurodegeneration, angiogenesis, invasion/metastasis and wound healing (Wyman et al., 2000, Immunol Today 21(6) 260-4 and Gerard et al., 2001, Nat. Immunol. 2(2) 108-15).

PI3-kinase activation, is therefore believed to be involved in a range of cellular responses including cell growth, differentiation, migration and apoptosis (Parker et al., 1995, Current Biology, 5, 577-99; Yao et al., 1995, Science, 267, 2003-05).

Recent biochemical studies revealed that, Class I PI3Ks (e.g. Class IB isoform PI3Kγ) are dual-specific kinase enzymes, i.e. they display both lipid kinase activity (phosphorylation of phospho-inositides) as well as protein kinase activity, as they are able to induce the phosphorylation of other protein as substrates, including auto-phosphorylation as intra-molecular regulatory mechanism.

PI3Ks appear to be involved in a number of aspects of leukocyte activation. A p85-associated PI3-kinase activity has been shown to physically associate with the cytoplasmic domain of CD28, which is an important co-stimulatory molecule for the activation of T-cells in response to antigen. These effects are linked to increases in the transcription of a number of genes including interleukin-2 (IL-2), an important T cell growth factor (Fraser et al., 1991, Science, 251, 313-16). Mutation of CD28 such that it can longer interact with PI3-kinase leads to a failure to initiate IL-2 production, suggesting a critical role for PI3-kinase in T cell activation.

Cellular processes in which PI3Ks play an essential role include suppression of apoptosis, reorganization of the actin skeleton, cardiac myocyte growth, glycogen synthase stimulation by insulin, TNFa-mediated neutrophil priming and superoxide generation, and leukocyte migration and adhesion to endothelial cells.

Recently, it has been described that PI3Kγ relays inflammatory signals through various G(i)-coupled receptors (Laffargue et al., 2002, Immunity 16(3) 441-51) and its central to mast cell function, stimuli in context of leukocytes, immunology includes cytokines, chemokines, adenosines, antibodies, integrins, aggregation factors, growth factors, viruses or hormones for example (Lawlor et al., 2001, J. Cell. Sci., 114 (Pt 16) 2903-10).

Two compounds, LY294002 and Wortmannin (cf. hereinafter), have been widely used as PI3-kinase inhibitors. These compounds are non-specific PI3K inhibitors, as they do not distinguish among the four members of Class I PI3-kinases.

IC50 values of Wortmannin against each of the various Class I PI3-kinases are in the range of 1-10 nM and IC50 values for LY294002 against each of these PI3-kinases are about 15-20 μM (Fruman et al., 1998, Ann. Rev. Biochem., 67, 481-507), also 5-10 mM on CK2 protein kinase and some inhibitory activity on phospholipases.

Wortmannin is a fungal metabolite which irreversibly inhibits PI3K activity by binding covalently to the catalytic domain of this enzyme. Inhibition of PI3K activity by wortmannin eliminates the subsequent cellular response to the extracellular factor (Thelen et al., 1994, Proc. Natl. Acad. Sci. USA, 91, 4960-64). Experiments with wortmannin, show that PI3K activity in cells of hematopoietic lineage, particularly neutrophils, monocytes, and other types of leukocytes, is involved in many of the non-memory immune response associated with acute and chronic inflammation.

Based on studies using Wortmannin, there is evidence that PI3-kinase function is also required for some aspects of leukocyte signalling through G-protein coupled receptors (Thelen et al., 1994, above). Moreover, it has been shown that Wortmannin and LY294002 block neutrophil migration and superoxide release.

Some results have indicated that PI3K inhibitors, for example, LY294002, can increase the in vivo antitumor activity of certain cytotoxic agents (e.g. paclitaxel) (Grant, 2003, Current Drugs, 6(10), 946-948).

However, in as much as these compounds do not distinguish among the various isoforms of PI3K, it remains unclear which particular PI3K isoform or isoforms are involved in these phenomena. Specific inhibitors against individual members of a family of enzymes provide valuable tools for deciphering functions of each enzyme as depending on the disease application, varying the degree of selectivity for PI3K isoforms can be of interest.

p110δ is expressed predominantly in cells of hemopoeitic origin such as leukocytes.

To assess the role of the d isoform of the p110 catalytic subunit of PI3Ks, PI3Kδ-null mice have been recently developed (Jou et al., 2002, Molecular and Cellular biology, 22(4), 8580-8591) and their specific immunological phenotype has been well characterized (Vanhaesebroeck et al., 2005, Trends in Biochemical Sciences, 30(4), 194-204). These experiments show that the PI3Kδ-null animals are viable and that a deficiency in PI3Kδ results in a very specific loss of the function of the B-cell antigen specific receptor complex, while signalling through the cytokine receptor complexes is unaffected (Jou et al., 2002, above).

It has been also shown that the inactivation of the p110δ isoform of PI3K in mast cells leads to defective stem cell factor-mediated in vitro proliferation, adhesion and migration and to impaired allergen-IgE-induced degranulation and cytokine release. Inactivation of p110δ protects mice against anaphylactic allergic responses, suggesting p110δ as a target for therapeutic intervention in allergy and mast-cell-related pathologies (Ali. et al., 2004, Nature, 431, 1007-1010).

Mast cells have emerged as a unique immune cell that could participate in a variety of inflammatory diseases in the nervous system (e.g. multiple sclerosis), skin, joints as well as cardiopulmonary, intestinal and urinary systems (Theoharides et al., 2004, J. of Neuroimmunology, 146, 1-12).

The high relevance of the PI3K pathway in some widely spread diseases stresses the need to develop inhibitors, including selective inhibitors, of PI3K isozymes, in order that the functions of each isozyme can be better characterized.

Recently, PI3K inhibitors have been developed: thiazole derivatives (WO 2005/021519; and WO 04/078754), thiazolidine derivatives (WO 2004/007491 and WO 2004/056820) and Quinazolinones derivatives (WO 03/035075).

Pyrido[3,2-d]pyrimidine derivatives with particular substitution pattern have been studied. EP 1277738 describes 4-morpholino-pyrido[3,2-d]pyrimidine derivatives substituted in positions 2, 6 and 7, involved in the Pi3K inhibition for the treatment of cancer. No indication is provided regarding the selectivity of these compounds. WO2008/023161 provides methylmorpholino pyrido[3,2-d]pyrimidine derivatives as MTOR inhibitors. The patent WO2006/069805 discloses pyrido[3,2-d]pyrimidine derivatives substituted in position 2, 4, 6 and/or 7 for the treatment of disorder of central nervous system and autoimmune disorder. WO 2006/087229 provide 2,4,6-trisubstituted pyrido[3,2-d]pyrimidine derivatives active against autoimmune and central nervous system disorders and cardiovascular diseases. The present invention provides morpholino pyrido[3,2-d]pyrimidine derivatives substituted in position 2, 6 and 8, and used as Pi3K modulators.

### SUMMARY OF THE INVENTION

According to one aspect of the invention, are provided pyridopyrimidine compounds. According to another aspect of the invention, are provided pyrimidopyrimidine compounds which are suitable for the treatment and/or prevention of disorders related to phosphoinositide-3-kinases, PI3Ks, such as PI3K alpha or PI3K gamma or PI3K delta or PI3K beta.

According to another aspect of the invention, are provided pyridopyrimidine compounds, which are able to modulate, especially inhibit the activity or function of phosphoinositide-3-kinases, PI3Ks in disease states in mammals, especially in humans.

According to another aspect of the invention, are provided methods for the treatment and/or prevention of disorders selected from auto-immune, inflammatory disorders, cardiovascular diseases, neurodegenerative disorders, bacterial and viral infections, allergy, asthma, pancreatitis, multi-organ failure, kidney diseases, platelet aggregation, cancer, transplantation, sperm motility, erythrocyte deficiency, graft rejection, lung injuries, respiratory diseases and ischemic conditions.

According to another aspect of the invention, is provided a process for the synthesis of pyridopyrimidine compounds

### DETAILED DESCRIPTION OF THE INVENTION

In one embodiment, the invention provides pyridopyrimidine compounds of Formula (I):

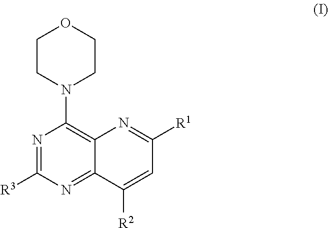

### Wherein

R¹ denotes H, perfluoroalkyl, —NH₂, —NA₂, A, —NH-A, —NH—(CH₂)ₚ-A, —SO-A, SO₂-A, —COOR^{T}, —(CH₂)ₚ—OR^{T}, —(CH₂)ₚ—SR^{T}, —COA, —CO-Het, —CO—N(H)₂₋ₘ(A)ₘ; —SO—N(H)₂₋ₘ(A)ₘ, SO₂—N(H)₂₋ₘ(A)ₘ, —(CH₂)ₚ—N(H)₂₋ₘ(A)ₘ, —CO—NH—(CH₂)ₚ—N(H)₂₋ₘ(A)ₘ, —(CH₂)ₚ—NH—(CH₂)ₚ—N(H)₂₋ₘ(A)ₘ, Ar, Het,
R² denotes H, Hal, CF₃, A, Ar, Het, SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚHet, —(CH₂)ₚ—N(H)₂₋ₘ(A)ₘ,
R³ denotes Hal, Ar, OA, SA, —SOA, —SO₂A, —NH—SO₂A, CF₃, —CN, A, —NH—SO₂Ar, or if at least one of R¹ or R² are different from H, R³ also denotes Het.
R^{T} denotes H, A, Ar, Het,
Ar denotes a monocyclic or bicyclic, aromatic carbocyclic ring having 6 to 14 carbon atoms, which is unsubstituted or monosubstituted, disubstituted or trisubstituted by, Hal, CF₃, OCF₃, NO₂, CN, perfluoroalkyl, A, OA, NH₂, COH, CONH₂, —NHCOA, —NHSO₂A, —NHSO₂—N(H)₂₋ₘ(A)ₘ, N(H)_{1-q}A_{q}COA, N(H)_{1-q}A_{q}SO₂—N(H)₂₋ₘ(A)ₘ, —N(H)_{1-q}A_{q}CON(H)₂₋ₘ(A)ₘ, —COOA, —SO₂A, —SO₂N(H)₂₋ₘ(A)ₘ, —SO₂Het, —(CH₂)ₚ—N(H)₂₋ₘ(A)ₘ, —(CH₂)—OR^{T}, or disubstituted or trisubstituted by OH and 1 or 2 of above described substituents.
Het denotes a monocyclic or bicyclic saturated, unsaturated or aromatic heterocyclic ring having 1, 2, 3 or 4 N, O and/or S atoms which is unsubstituted or monosubstituted, disubstituted or trisubstituted by alkyl having 1 to 8 carbon atoms, alkoxy having 1 to 8 carbon atoms, Hal, CF₃, OCF₃, NO₂, CN, perfluoroalkyl, A, OA, OH, NH₂, COH, CONH₂, —NHCOA, —NHSO₂A, —NHSO₂—N(H)₂₋ₘ(A)ₘ, N(H)_{1-q}A_{q}COA, N(H)_{1-q}A_{q}SO₂—N(H)₂₋ₘ(A)ₘ, —N(H)_{1-q}A_{q}CON(H)₂₋ₘ(A)ₘ, —COOA, —SO₂A, —SO₂N(H)₂₋ₘ(A)ₘ, —SO₂Het, —(CH₂)ₚ—N(H)₂₋ₘ(A)ₘ, —(CH₂)ₚ—OR^{T},
m denotes 0, 1 or 2;
p denotes 0, 1, 2, 3 or 4;
q denotes 0 or 1;
A is a branched or linear alkyl having 1 to 12 C-atoms, wherein one or more, preferably 1 to 7 H-atoms may be replaced by Hal, Ar, Het, OR⁶, —CN, —COOalkyl or N(R⁶)₂ and wherein one or more, preferably 1 to 7 non-adjacent CH₂-groups, excluding the carbon atom which is linked to the rest of the molecule, may be replaced by O, NR⁶ or S and/or by —CH═CH— or —C≡C— groups, or denotes cycloalkyl or cycloalkylalkylen having 3-7 ring C atoms;
R⁶ is H, A, —(CH₂)ₚ—N(H)₂₋ₘ(A)ₘ, —(CH₂)ₚ—OA; CH₂NH₂,
   and pharmaceutically acceptable derivatives, solvates, tautomers, salts and stereoisomers thereof, including mixtures thereof in all ratios.

The following compound is used as an intermediate in the patent application DE 2208534 and is therefore excluded from the scope of the current subject matter.

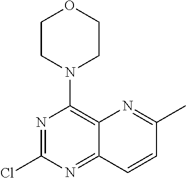

In a second embodiment, the invention relates to compounds of formulae (I-a)

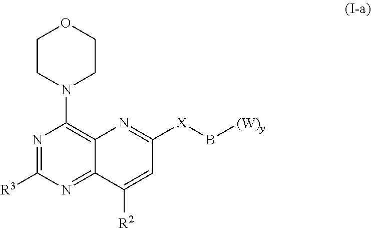

### Wherein

R², R³, m and p are as defined above
X denotes CO, CS, or CH₂,
B denotes O, N, S, SO, SO₂ or a bond,
W denotes H, A, —(CH₂)ₚ—N(H)₂₋ₘ(A)ₘ, —(CH₂)ₚ—OA;
y is 1 or 2
   and pharmaceutically acceptable derivatives, solvates, tautomers, salts and stereoisomers thereof, including mixtures thereof in all ratios.

In another embodiment, the invention relates to the compounds of formula (I-b), (I-c) or (I-d)

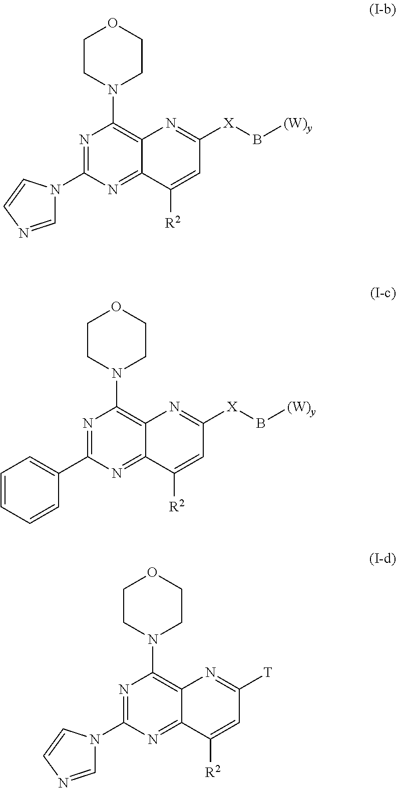

### Wherein

R², X, B, W, and y are as defined above
And T denotes Het.

In another embodiment, the invention relates to compounds of Formula (I-e):

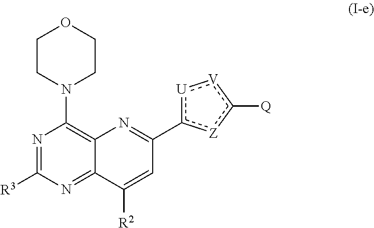

wherein R² is as defined above,

### R³ is Het

U, V and Z are independently of one another CH, O, S or N [symbol]
is a single or a double bond
Q is H, Hal, CF₃, (C₁-C₈)alkyl, SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚHet, —(CH₂)ₚ—OR^{T}, —(CH₂)ₚ—NR^{T},
Wherein R^{T}, m and p are as above defined,
and pharmaceutically acceptable derivatives, solvates, tautomers, salts and stereoisomers thereof, including mixtures thereof in all ratios.

Most preferably the invention relates to compounds of formula (I-f)

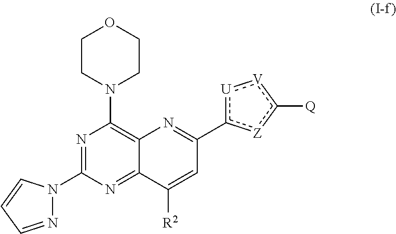

wherein V is as defined above,
R² denotes H, SA, N(H)₂₋ₘ(A)ₘ, Cl, N(A)-(CH₂)ₚ—OR^{T},
Q denotes (C₁-C₈)alkyl, —(CH₂)ₚ—N(H)₂₋ₘ(A)ₘ, —(CH₂)ₚ—OR^{T}, NH₂,
Z denotes N or O,
U denotes O, CH or N
And wherein R^{T}, m and p are as above defined,
and pharmaceutically acceptable derivatives, solvates, tautomers, salts and stereoisomers thereof, including mixtures thereof in all ratios.

Most preferably the invention relates to compounds of formula (I-g)

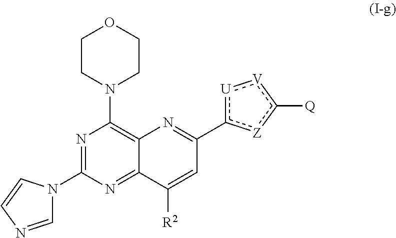

wherein V is as defined above,
R² denotes H, SA, N(H)₂₋ₘ(A)ₘ, Cl, N(A)-(CH₂)ₚ—OR^{T},
Q denotes (C1-C8)alkyl, —(CH₂)ₚ—N(H)₂₋ₘ(A)ₘ, —(CH₂)ₚ—OR^{T}, NH₂,
Z denotes N or O,
U denotes O, CH or N
And wherein R^{T}, m and p are as above defined,
and pharmaceutically acceptable derivatives, solvates, tautomers, salts and stereoisomers thereof, including mixtures thereof in all ratios.

Above and below, Me refers to a methyl group, Et refers to a ethyl group.

The formula (I) and related formulae also encompasses mixtures of the compounds of the formula (I), for example mixtures of two diastereomers, for example in the ratio 1:1, 1:2, 1:3, 1:4, 1:5, 1:10, 1:100 or 1:1000.

These are particularly preferably mixtures of stereoisomeric compounds.

Very particularly, compounds of formula (I) are the more preferred, the more preferred substituents they carry.

R¹ preferably denotes H, —CH₃, Et, —CH₂OH, —CH₂OMe, —CH₂OCH(CH₃)₂, —CH₂NMe₂, —CH₂NHMe, —CH₂SMe, —CH₂SO₂Me, —CH₂—(NH)—(CH₂)₂—NMe₂, —CO—NH—(CH₂)₂—NMe₂, —CONMe₂, —CONHMe, —CONH₂, —CO₂Me, —CO₂Et, —CO₂H,

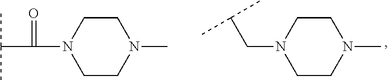

or a group

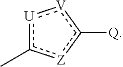

If R¹ is

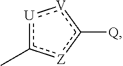

then it has preferrably one of the following meanings:

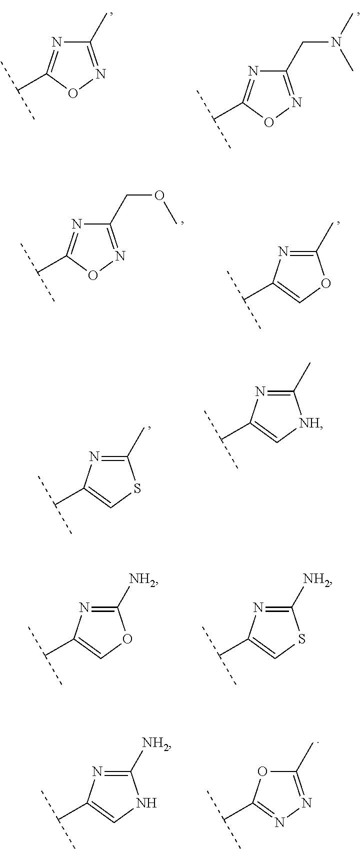

R² preferably denotes H, NH—(CH₂)₂—NMe₂, —NMe₂, —NMe(CH₂)₂OMe, Cl, —SMe, —SO₂Me, Ph, —CH₂—NH—(CH₂)₂—NMe₂, —NH—(CH₂)₂—OMe, —CH₂—NMe₂,

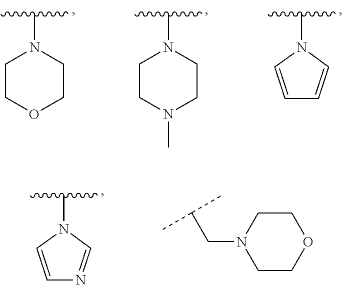

R³ preferably denotes Ar or Het,

and more preferably one of the following groups: methyl, NMe₂, NEt₂, —NH(CH₂)₃—CH₃, —O(CH₂)₂—NMe₂, SMe, OMe, CN, Cl,

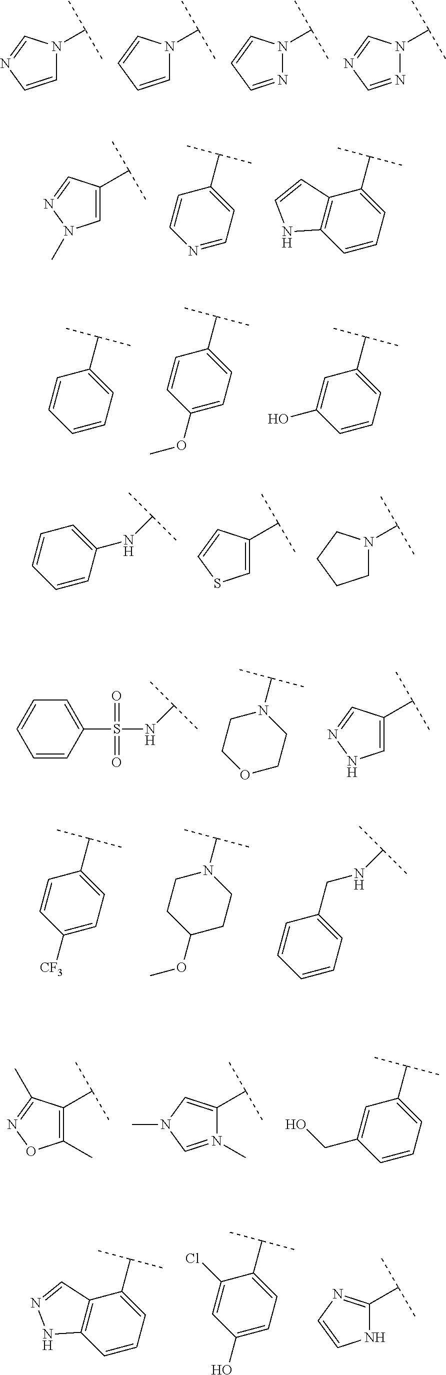

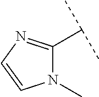

Alkyl denotes a carbon chain having 1, 2, 3, 4, 5, 6, 7, 8, 9, 10, 11, or 12 carbon atoms. Alkyl preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethylpropyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-tri-methylpropyl, furthermore preferably, for example, trifluoromethyl, pentafluoroethyl or 1,1,1-trifluoroethyl.

Cycloalkyl are cyclic alkyl containing 3 to 12 carbon atomes.

Cycloalkyl preferably denotes cyclopropyl, cyclobutyl, cyclopentyl, cyclohexyl or cycloheptyl.

Cycloalkylalkylene is a cycloalkyl group bond to the rest of the molecule via a carbon chain and having 1, 2, 3, 4, 5, 6, 7, 8, 9, 10, 11, 12, 13, 14, 15, 16, 17, or 18 carbon atoms. Cycloalkylalkylene preferably denotes cyclopropylmethylene, cyclobutylmethylene, cyclopentylmethylene, cyclohexylmethylene or cycloheptylmethylene.

Alkylene is a bivalent carbon chain having 1, 2, 3, 4, 5, 6, 7, 8, 9, 10, 11, or 12 carbon atoms. Alkylene is preferably methylene, ethylene, propylene, butylene, pentylene or hexylene, furthermore branched alkylene.

Perfluoroalkyl denotes an alkyl chain having 1, 2, 3, 4, 5, 6, 7, 8, 9, 10, 11, or 12 carbon atoms and wherein all the hydrogen atoms are replaced by F atoms, preferably denotes CF₃.

Fluoroalkyl denotes an alkyl chain having 1, 2, 3, 4, 5, 6, 7, 8, 9, 10, 11, or 12 carbon atoms and wherein one or more of the hydrogen atoms are replaced by F atoms.

Hal denotes Cl, Br, I, F and preferably F, Cl or Br.

Alkoxy is branched or linear and preferably denotes a group —O—(CH₂)ₚ—CH₃ wherein p is as above defined. Most preferably alkoxy is Methoxy or Ethoxy.

Carboxy denotes a group —COOH.

Hydroxy denotes an —OH group

Carboxyalkyl denotes an ester group, preferably an alkyl ester, such as COOMe or COOEt.

Sulfonyl denotes a group —SO₂—

Alkylsulfonyl denotes a group —SO₂-alkyl, preferably methylsulfonyl or ethylsulfonyl.

Acyl denotes a group —C(O)R, wherein R can be A, Ar, Het as defined above. Preferably Acyl denotes acetyl (—C(O)CH₃).

Amino denotes the group —NR′R″ where each R′, R″ is independently hydrogen, (C1-C8)alkyl, Ar, Het or A. R′ and R″, together with the nitrogen atom to which they are attached, can optionally form a Het group. R′ and R″, together with the nitrogen atom to which they are attached, preferrably form a 5-membered unsaturated or aromatic heterocyclic ring having 1, 2, 3, 4, heteroatoms selected in the group of N, O, and S.

Alkylamine denotes the group —(CH₂)ₚ—NR′R″ wherein each R′, R″ is independently hydrogen, alkyl, Ar, Het or A, and wherein p is as defined above. R′ and R″, together with the nitrogen atom to which they are attached, can optionally form a Het group. R′ and R″, together with the nitrogen atom to which they are attached, preferrably form a 5-membered unsaturated or aromatic heterocyclic ring having 1, 2, 3, 4, heteroatoms selected in the group of N, O, and S.

Amido refers to the group —C(O)NR′R″ where each R′, R″ is independently hydrogen, alkyl, Ar, Het or A, and where R′ and R″, together with the nitrogen atom to which they are attached, can optionally form a Het group. R′ and R″, together with the nitrogen atom to which they are attached, preferrably form a 5-membered unsaturated or aromatic heterocyclic ring having 1, 2, 3, 4, heteroatoms selected in the group of N, O, and S.

Ar denotes preferably a monocyclic or bicyclic, aromatic carbocyclic ring having 6 to 14 carbon atoms, which is unsubstituted or monosubstituted, disubstituted or trisubstituted by alkyl having 1 to 8 carbon atoms, alkoxy having 1 to 8 carbon atoms, Hal, CF₃, OCF₃, NO₂, CN, perfluoroalkyl, A, OA, amino, CONH₂, —NHCOA, —NHSO₂—N(H)₂₋ₘ(A)ₘ, —COOA, —SO₂A, —SO₂N(H)₂₋ₘ(A)ₘ, —SO₂Het

More particulary, Ar is unsubtituted or:

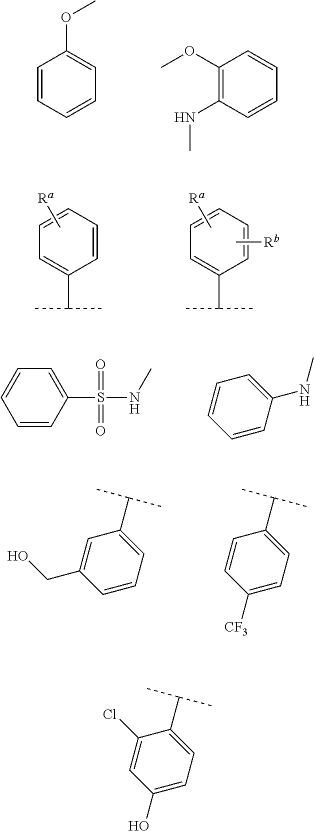

wherein R^{a} and R^{b} denote independently each other Ar, Het, OA or A. R^{a} preferably denotes OA, —SO₂NHA, —SO₂N(H)₂₋ₘ(A)ₘ, NHSO₂A, or —SO₂-A, NHA, and R^{b} is preferably —CH₂OH, Cl or CF₃.

Het is preferably a 6 to 14 membered ring system having 1, 2, 3 or 4 heteroatoms and denotes, not withstanding further substitutions, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, indazolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-iso-quinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxane-6-yl, 2,1,3-benzothiadiazol-4- or -5-yl or 2,1,3-benzoxadiazol-5-yl. The heterocyclic radicals may also be partially or fully hydrogenated.

Het can thus also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxaneyl, 1,3-dioxane-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

Het very particularly denotes one of the following groups:

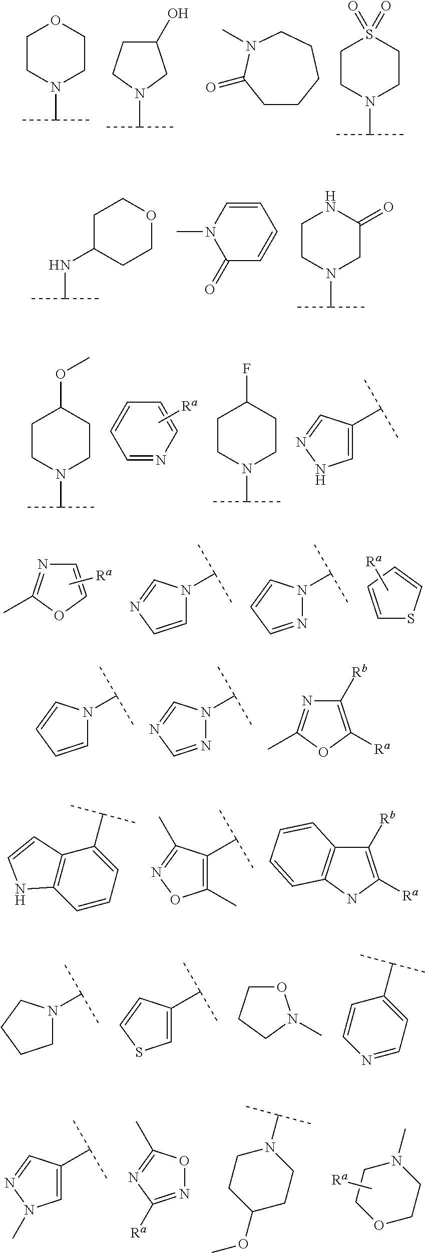

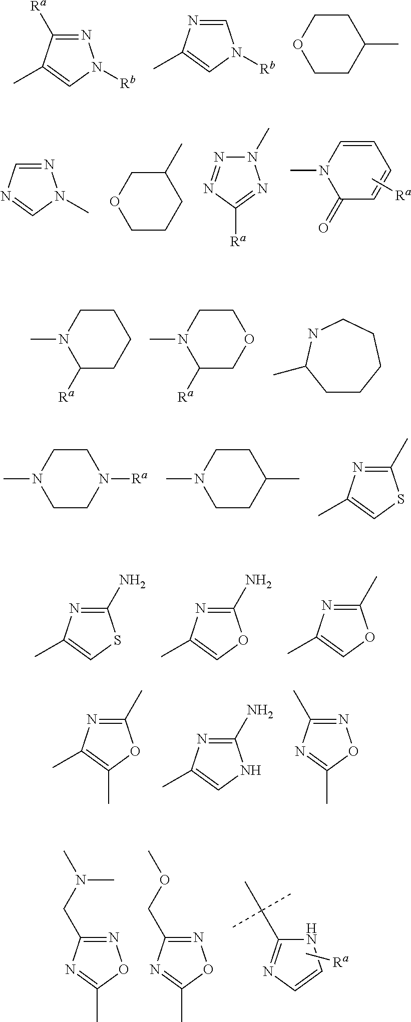

Wherein R^{a} and R^{b} denote independently from one another H, Ar, Het, OA or A. R^{a} preferably denotes OH, Cl or CF₃ and R^{b} is preferably OA, —SO₂NHA, —SO₂N(A)₂, NHSO₂A, or —SO₂-A

In one embodiment, the group A denotes a branched or linear alkyl having 1 to 12 C-atoms, wherein one or more, preferably 1 to 7 H-atoms may be replaced by Hal, Ar, Het, OR⁶, —CN, —COOalkyl or N(R⁶)₂ and wherein one or more, preferably 1 to 7 non-adjacent CH₂-groups may be replaced by O, NR⁶ or S and/or by —CH═CH— or —C≡C— groups, or denotes cycloalkyl or cycloalkylalkylen having 3-7 ring C atoms;

In another embodiment, the group A denotes a branched or linear alkyl having 1 to 12 C-atoms, wherein one or more, preferably 1 to 7 H-atoms are replaced by Hal, Ar, Het, OR⁶, —CN, —COOalkyl or N(R⁶)₂ or wherein one or more, preferably 1 to 7 non-adjacent CH₂-groups are replaced by O, NR⁶ or S and/or by —CH═CH— or —C≡C— groups, or denotes cycloalkyl or cycloalkylalkylen having 3-7 ring C atoms;

In a specific embodiment, the present invention provides compounds of formula (I)

### Wherein

R¹ denotes H, perfluoroalkyl, —NH₂, —NA₂, A, —NH-A, —NH—(CH₂)ₚ-A, —SO-A, SO₂-A, —COOR^{T}, —(CH₂)ₚ—OR^{T}, —(CH₂)—SR^{T}, —COA, —CO-Het, —CO—N(H)₂₋ₘ(A)ₘ; —SO—N(H)₂₋ₘ(A)ₘ, SO₂—N(H)₂₋ₘ(A)ₘ, —(CH₂)ₚ—N(H)₂₋ₘ(A)ₘ, —CO—NH—(CH₂)ₚ—N(H)₂₋ₘ(A)ₘ, —(CH₂)ₚ—NH—(CH₂)ₚ—N(H)₂₋ₘ(A)ₘ, Ar, Het
R² denotes H, Hal, CF₃, A, Ar, Het, SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚHet, —(CH₂)ₚ—N(H)₂₋ₘ(A)ₘ,
And wherein R³, R^{T}, m, p, and q are as above defined.

In another specific embodiment, the present invention provides compounds of formula (I) wherein

R¹ denotes perfluoroalkyl, —NH₂, —NA₂, A, —NH-A, —NH—(CH₂)-A, —SO-A, SO₂-A, —COOR^{T}, —(CH₂)ₚ—OR^{T}, —(CH₂)ₚ—SR^{T}, —COA, —CO—N(H)₂₋ₘ(A)ₘ; —SO—N(H)₂₋ₘ(A)ₘ, SO₂—N(H)₂₋ₘ(A)ₘ, —(CH₂)ₚ—N(H)₂₋ₘ(A)ₘ, —CO—NH—(CH₂)ₚ—N(H)₂₋ₘ(A)ₘ, —(CH₂)ₚ—NH—(CH₂)ₚ—N(H)₂₋ₘ(A)ₘ, Ar, Het,
or, when R³ is Het, OA, SA, —SOA, —SO₂A, —NH—SO₂A, CF₃, —CN, A, —NH—SO₂Ar, R¹ also denotes H,
or, if R³ is Ar, Het, OA, SA, —SOA, —SO₂A, —NH—SO₂A, CF₃, —CN, A, —NH—SO₂Ar, R¹ also denotes —CO-Het,
   R² denotes Hal, CF₃, A, Ar, Het, SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚHet, —(CH₂)ₚ—N(H)₂₋ₘ(A)ₘ, or if R³ is Ar, Het, OA, SA, —SOA, —SO₂A, —NH—SO₂A, CF₃, —CN, A, —NH—SO₂Ar, R² also denotes H, and wherein R³, R^{T}, m, p, and q are as above defined.

In another specific embodiment, the invention provides compounds of formula (I) wherein
R¹ denotes A, COA, COOA, CSA, CO—NH-A, wherein A is as above defined,
R² denotes H, Hal, Ar, Het, NMe₂, OA, SA, SO₂A, —NA-(CH₂)ₚ—NR^{T}₂, —NA-(CH₂)—OR^{T},
wherein p and R^{T} are as above defined
R³ denotes Cl, Ar, Het;

In another specific embodiment the invention provides compounds of formula (I) wherein
R¹ denotes A, COA, COOA, CO—NH-A, wherein A is as above defined,
R² denotes H, Cl, Het, NMe₂, OA, SA, —NH—(CH₂)ₚ—NR^{T}₂, —NA-(CH₂)ₚ—OR^{T}, wherein p and R^{T} are as above defined.
R³ denotes Cl, Ar, Het;

In another specific embodiment the invention provides compounds of formula (I) wherein
R¹ denotes A, COA, COOA, CO—NH-A, wherein A is as above defined,
R² denotes H, Cl, Het, NMe₂, OMe, SMe, —NH—(CH₂)₂—NR^{T}₂, —NA-(CH₂)₂—OR^{T}, wherein
R^{T} is H or (C1-C8)alkyl,
R³ denotes Ar, Het wherein Ar and Het are unsubstituted.

In another specific embodiment the invention provides compounds of formula (I) wherein
R¹ denotes A, COOMe, CO—NHMe, CO—NMe₂, CH₂OH, CH₂OMe,
R² denotes H, Cl, morpholine, N-methyl-piperazine, NMe₂, OMe, SMe, —NH—(CH₂)₂—NR^{T}₂, —NMe-(CH₂)₂—OR^{T}, wherein R^{T} is H or (C1-C8)alkyl,
R³ denotes Ar, Het wherein Ar is unsubstituted phenyl and Het is unsubstituted imidazolyl, pyrazoline, pyrrolidine, pyridinyl or morpholine.

In another specific embodiment, the invention provides compounds of formula (I-a) wherein
R² denotes H, Hal, Ar, Het, NMe₂, OA, SA, SO₂A, —NA-(CH₂)ₚ—NR^{T}₂, —NA-(CH₂)ₚ—OR^{T},
wherein A and p are as above defined,
R³ is Hal, Ar, Het,
B is O, N, or S,

In another specific embodiment, the invention provides compounds of Formula (I-a) wherein
R² denotes H, Cl, Het, NMe₂, OA, SA, —NH—(CH₂)ₚ—NR^{T}₂, —NA-(CH₂)ₚ—OR^{T}, wherein p is as above defined.
R³ is Hal, Ar, Het,
B is O, N, or S,

In another specific embodiment, the invention provides compounds of Formula (I-a) wherein
R² denotes H, Cl, Het, NMe₂, OMe, SMe, —NH—(CH₂)₂—NR^{T}₂, —NA-(CH₂)₂—OR^{T}, wherein
R^{T} is H or (C1-C8)alkyl,
R³ is Hal, Ar, Het,
B is O, N, or S,

In another specific embodiment, the invention provides compounds of Formula (I-a) Wherein
R² denotes H, Cl, morpholine, N-methyl-piperazine, NMe₂, OMe, SMe, —NH—(CH₂)₂—NR^{T}₂, —NMe-(CH₂)₂—OR^{T}, wherein R^{T} is H or (C1-C8)alkyl,
R³ is Cl, Ar, Het,
B is O or N
W is H or (C1-C8)alkyl

In another specific embodiment, the invention provides compounds of Formula (I-e) wherein
R² denotes H, Hal, Ar, Het, NMe₂, OA, SA, SO₂A, —NA-(CH₂)ₚ—NR^{T}₂, —NA-(CH₂)—OR^{T}
R³ is Het
U, V and Z are independently of one another CH, O, S or N
Q is H, (C1-C8)alkyl; OA, OH, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚ—OR^{T}, —(CH₂)—NR^{T},

In another specific embodiment, the invention provides compounds of Formula (I-e) wherein
R² denotes H, Cl, Het, NMe₂, OMe, SMe, —NH—(CH₂)₂—NR^{T}₂, —NA-(CH₂)₂—OR^{T}, wherein
R^{T} is H or (C1-C8)alkyl,
R³ is pyrazoline, imidazolyl, methyl-imidazolyl,
U, V and Z are independently of one another CH, O, or N
Q is H, (C1-C8)alkyl, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚ—OR^{T}, —(CH₂)—NR^{T}, wherein R^{T} is H or (C1-C8)alkyl

Preference is given to the compounds of the present invention selected from the following group E-1 to E-72:

| Example | Structure |
|---|---|
| E-1 | 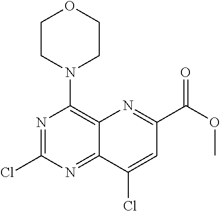 |
| E-2 | 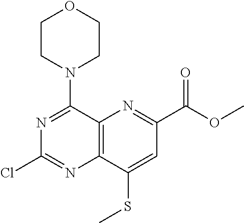 |
| E-3 | 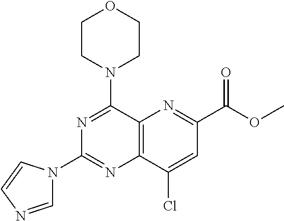 |
| E-4 | 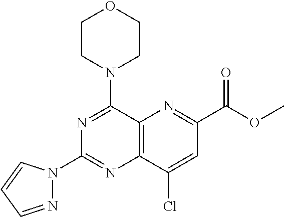 |
| E-5 | 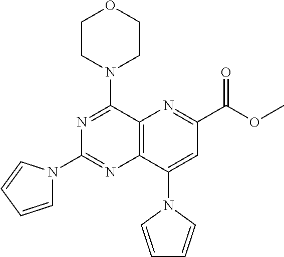 |
| E-6 | 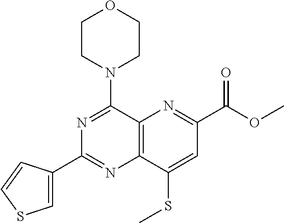 |
| E-7 | 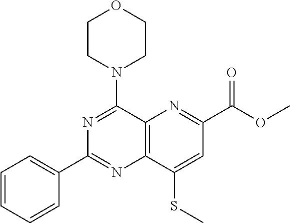 |
| E-8 | 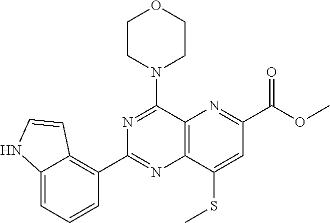 |
| E-9 | 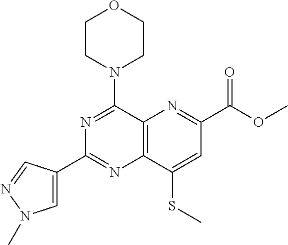 |
| E-10 | 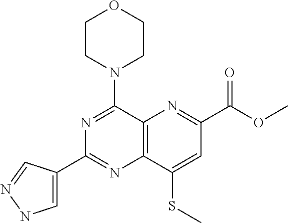 |
| E-11 | 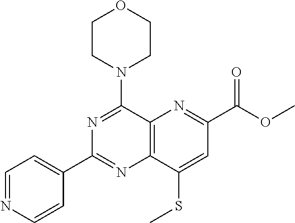 |
| E-12 | 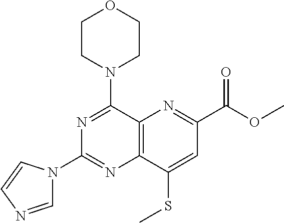 |
| E-13 | 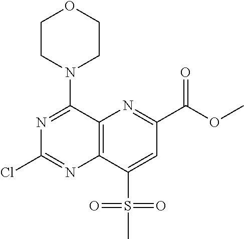 |
| E-14 | 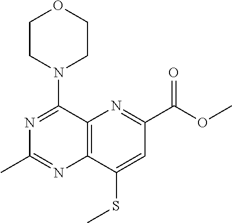 |
| E-15 | 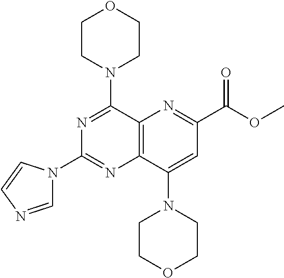 |
| E-16 | 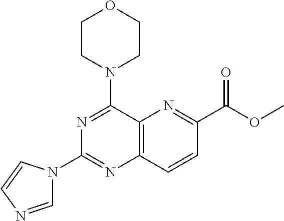 |
| E-17 | 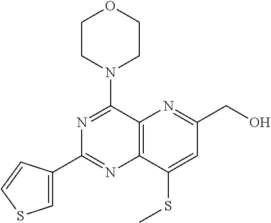 |
| E-18 | 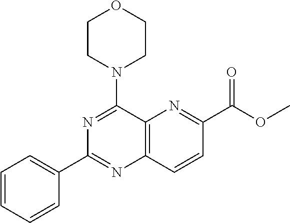 |
| E-19 | 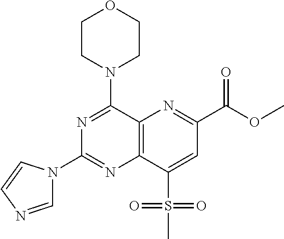 |
| E-20 | 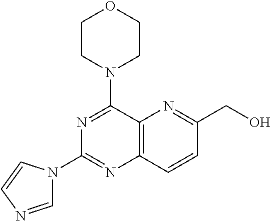 |
| E-21 | 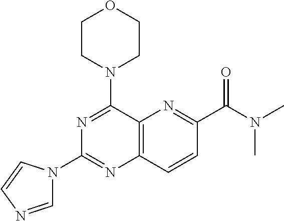 |
| E-22 | 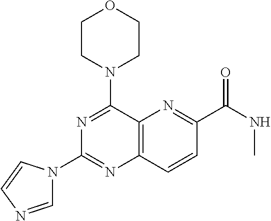 |
| E-23 | 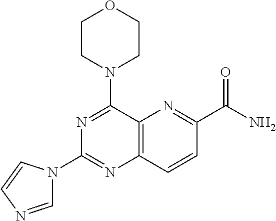 |
| E-24 | 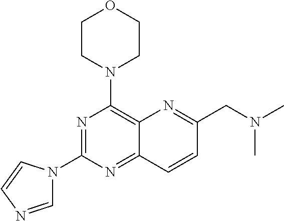 |
| E-25 | 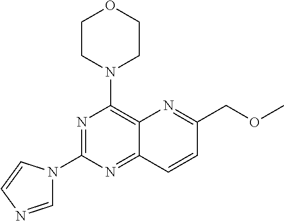 |
| E-26 | 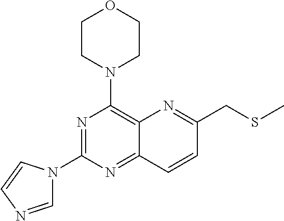 |
| E-27 | 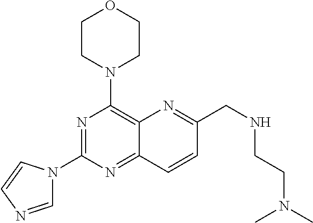 |
| E-28 | 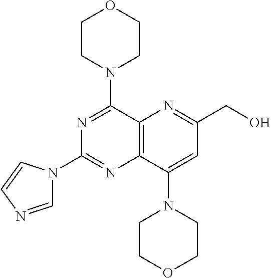 |
| E-29 | 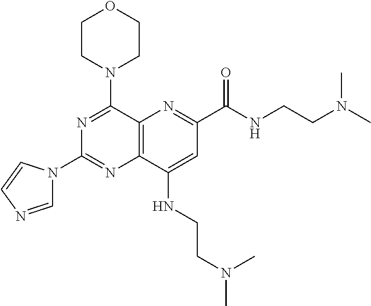 |
| E-30 | 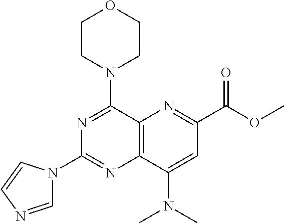 |
| E-31 | 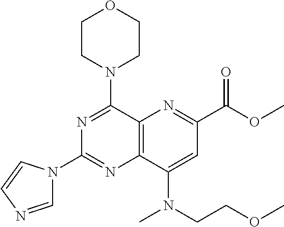 |
| E-32 | 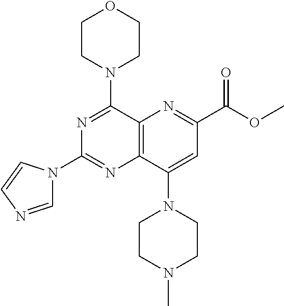 |
| E-33 | 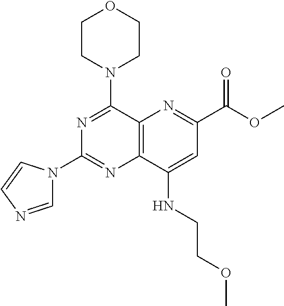 |
| E-35 | 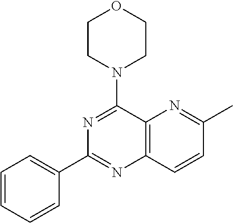 |
| E-36 | 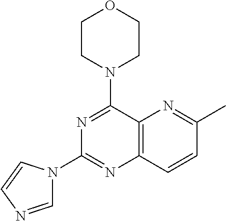 |
| E-37 | 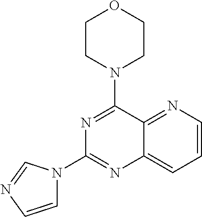 |
| E-38 | 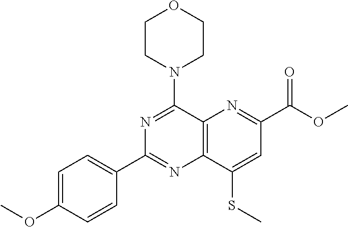 |
| E-39 | 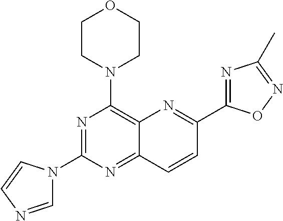 |
| E-40 | 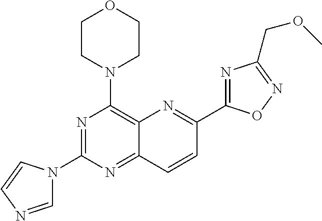 |
| E-41 | 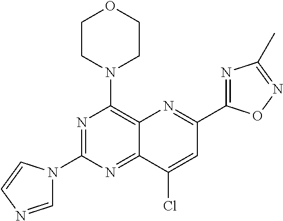 |
| E-42 | 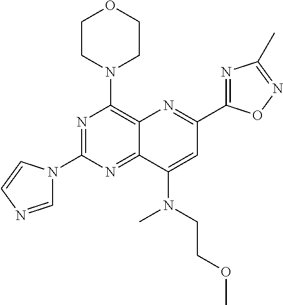 |
| E-43 | 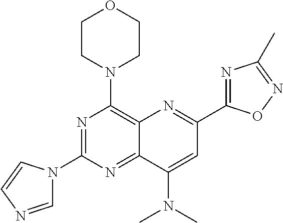 |
| E-44 | 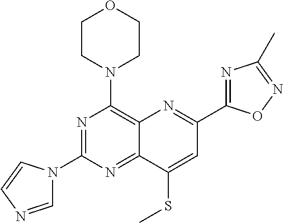 |
| E-45 | 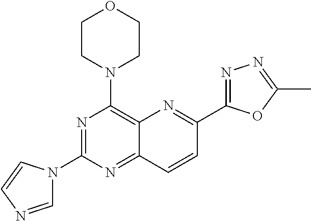 |
| E-46 | 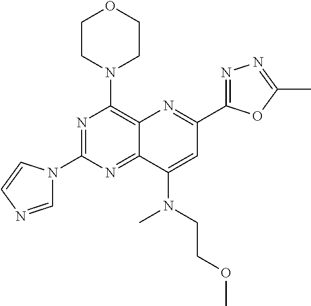 |
| E-47 | 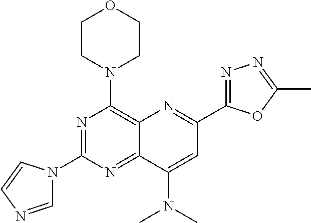 |
| E-48 | 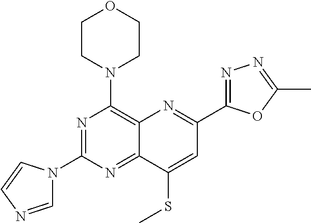 |
| E-49 | 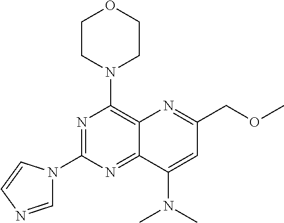 |
| E-50 | 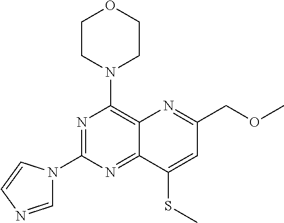 |
| E-51 | 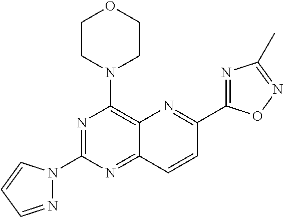 |
| E-52 | 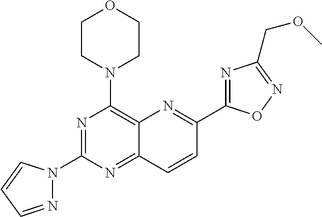 |
| E-53 | 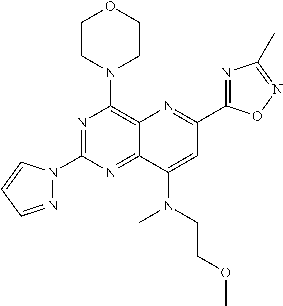 |
| E-54 | 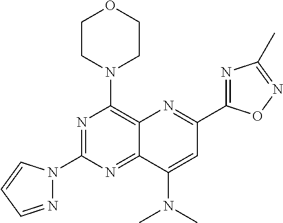 |
| E-55 | 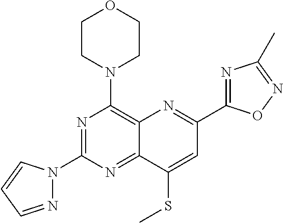 |
| E-56 | 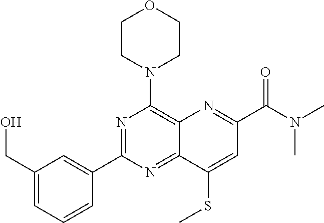 |
| E-57 | 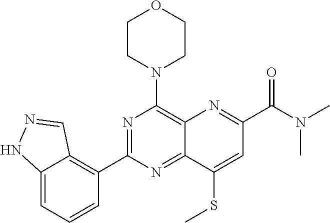 |
| E-58 | 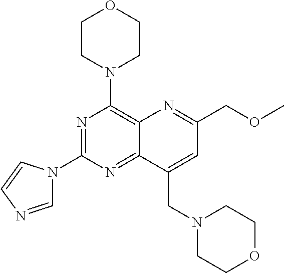 |
| E-59 | 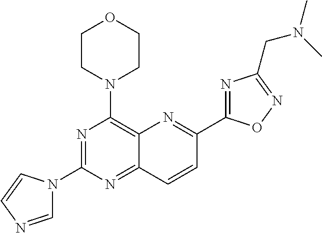 |
| E-60 | 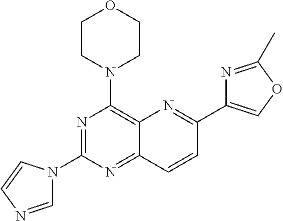 |
| E-61 | 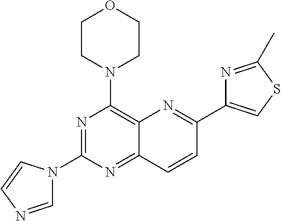 |
| E-62 | 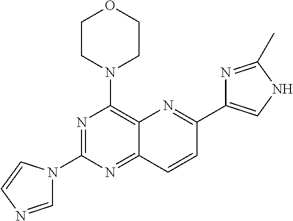 |
| E-63 | 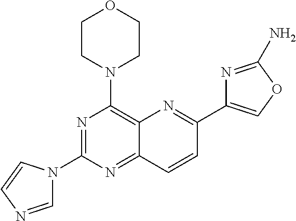 |
| E-64 | 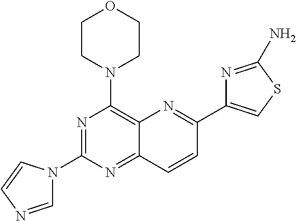 |
| E-65 | 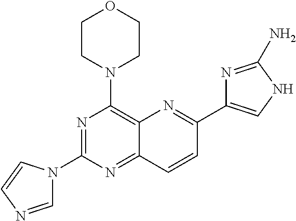 |
| E-66 | 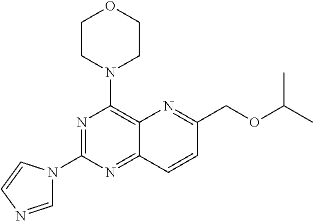 |
| E-67 | 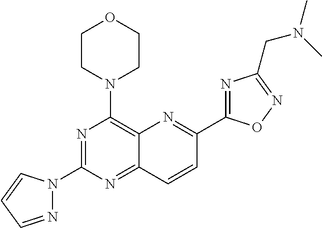 |
| E-68 | 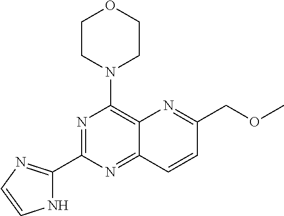 |
| E-69 | 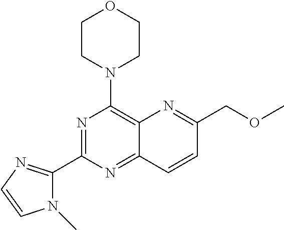 |
| E-70 | 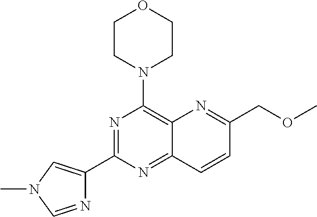 |
| E-71 | 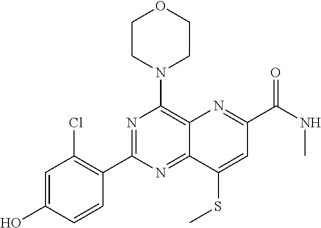 |
| E-72 | 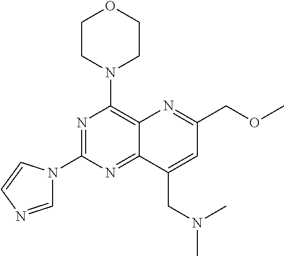 |

and pharmaceutically acceptable derivatives, solvates, tautomers, salts and stereoisomers thereof, including mixtures thereof in all ratios.

### Synthesis of Compounds of the Invention

The pyridopyrimidine compounds according to formula (I) may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

The following abbreviations refer respectively to the definitions below:

aq. (aqueous), g (gram), L (liter), mg (milligram), MHz (Megahertz), μM (micromolar) min (minute), mm (millimeter), mmol (millimole), mM (millimolar), m.p. (melting point), eq. (equivalent), mL (milliliter), μL (microliter), ACN (acetonitrile), br s (broad singlet), CDI (1,1′-carbonyldiimidazole), d (doublet), dba (dibenzylideneacetone), DCM (dichloromethane), DIBAL (di-isobutyl aluminum hydride), DIEA (diisopropylethyl-amine), DMF (dimethylformamide), DMSO (dimethylsulfoxide), DMSO-d₆ (deuterated dimethylsulfoxide), EDC (1-(3-dimethyl-amino-propyl)-3-ethylcarbodiimide hydrochloride), ESI (electro-spray ionization), Et₂O (diethyl ether), EtOH (ethanol), HATU ((2-(7-Aza-1H-benzotriazole-1-yl)-1,1,3,3-tetramethyluronium hexafluorophosphate)), HMDS (hexamethyldisilazane), HPLC (high performance liquid chromatography), LC (liquid chromatography), LDA (lithium di-isopropyl amide), LG (leaving group), m (mutilplet), MeOH (methanol), m-CPBA (3-chloroperbenzoic acid), MS (mass spectrometry), NMR (nuclear magnetic resonance), Ph (phenyl), q (quadruplet), quint (quintuplet), RT (retention time), s (singlet), SPE (solid phase extraction), t (triplet), TBTU (O-benzotriazole-1-yl-N,N,N′,N′-tetramethyluronium tetrafluoroborate), TEA (triethylamine), TFA (trifluoroacetic acid), THF (tetrahydrofuran), UV (ultraviolet).

Depending on the nature of R¹, R² and R³ different synthetic strategies may be selected for the synthesis of compounds of Formula (I). In the process illustrated in the following schemes R¹, R² and R³ areas above-defined in the description.

In general, the pyridopyrimidine compounds according to Formula (I) of this invention may be prepared from readily available starting materials. If such starting materials are not commercially available they may be prepared by standard synthetic techniques. The following general methods and procedures described hereinafter in the examples may be employed to prepare compounds of Formula (I).

Generally, compounds of Formula (I-a) wherein R² and R³ are as above defined, and X is —CH₂— and B is O, N or S can be prepared in two steps from the corresponding alcohols of Formula A, wherein R², R³ are as above defined and X is —CH₂—, by transforming the hydroxyl function into a leaving group (LG) such as an alkylsulfonate, an arylsulfonate or an halogen and further reacting this intermediate with an alcohol (or a salt thereof), an amine or a thiol (or a salt thereof) in the presence or the absence of a base such as TEA or DIEA in an appropriate solvent such as DCM, THF, dioxane, DMF, DMA or a mixture thereof (Scheme 1).

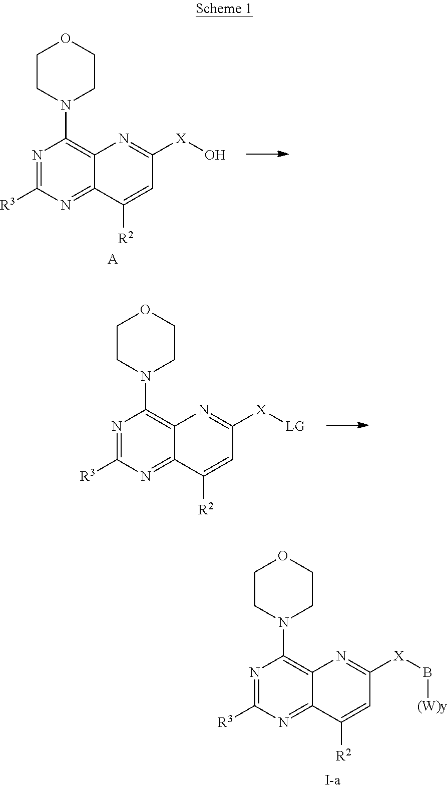

Preferrably, the method can be used for preparing the following compounds of Formula (I-a) selected below:
1-[2-(1H-Imidazol-1-yl)-4-morpholin-4-ylpyrido[3,2-d]pyrimidin-6-yl]-N,N-dimethylmethanamine
2-(1H-Imidazol-1-yl)-6-(methoxymethyl)-4-morpholin-4-ylpyrido[3,2-c]pyrimidine
2-(1H-Imidazol-1-yl)-6-[(methylthio)methyl]-4-morpholin-4-ylpyrido[3,2-d]pyrimidine
N′-{[2-(1H-Imidazol-1-yl)-4-morpholin-4-ylpyrido[3,2-d]pyrimidin-6-yl]methyl}-N,N-dimethylethane-1,2-diamine

Alternatively, compounds of Formula (I-a) wherein R² and R³ are as above defined, and X is —CH₂— and B is O can be prepared from the corresponding alcohols of Formula A, wherein R², R³ are as above defined and X is —CH₂—, by deprotonation of the hydroxyl function using a base such as sodium hydride, LDA, lithium or potassium HMDS and by further reacting this intermediate with a group W-(LG) wherein LG is a leaving group. Preferably LG is an halide or a sulfonate. The reaction is preferably performed in DCM, THF, dioxane, DMF, DMA or a mixture thereof (Scheme 2).

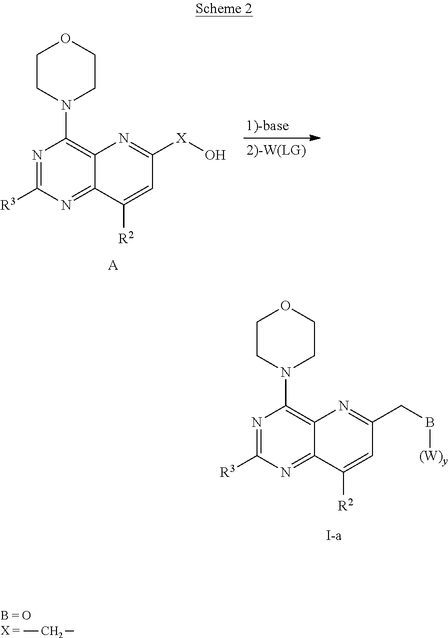

Compounds of formula (I-e) where U is O, V is N and Z is N can be prepared from compounds of Formula (II-a) where R² and R³ are as above defined, in a 2-step protocol as outlined in Scheme 3. The first step consists in the coupling of a carboxylic acid of formula (II-a) with an amidoxime of formula Q-C(NH₂)═NHOH. General protocols for such coupling are given below in the examples, using conditions and methods well known to those skilled in the art to prepare an O-substituted amidoximes from a carboxylic acid and an amidoxime, with standard coupling agents, such as but not limited to CDI, EDC, HATU, TBTU, in the presence or absence of bases such as TEA, DIEA, NMM in a suitable solvent such as DCM, ACN, THF or DMF, at a temperature rising from about 20° C. to about 50° C., preferably at RT, for a few hours, e.g. one hour to 24 h. Alternatively, a carboxylic acid derivative (e.g. acyl chloride) may be coupled with the amidoxime, using conditions and methods well known to those skilled in the art, in the presence of bases such as TEA, DIEA, NMM in a suitable solvent such as DCM, THF or DMF, at a temperature rising from about 20° C. to about 50° C., preferably at RT, for a few hours, e.g. one hour to 24 h (Scheme 3). The second step consists of the cyclization and dehydration of the O-substituted amidoximes to form the oxadiazole (I-e). Conditions are given below in the examples, using methods well known to those skilled in the art to prepare oxadiazole, such as heating at temperature rising from RT to about 150° C., typically 110° C., using possibly a microwave oven, for a time comprised between 15 minutes and 24 hours, preferably for 30 min, in a suitable solvent or mixture of solvents such as toluene, ACN, THF, Pyridine, DMF, in the presence or absence of a base such as DIEA, TEA or pyridine.

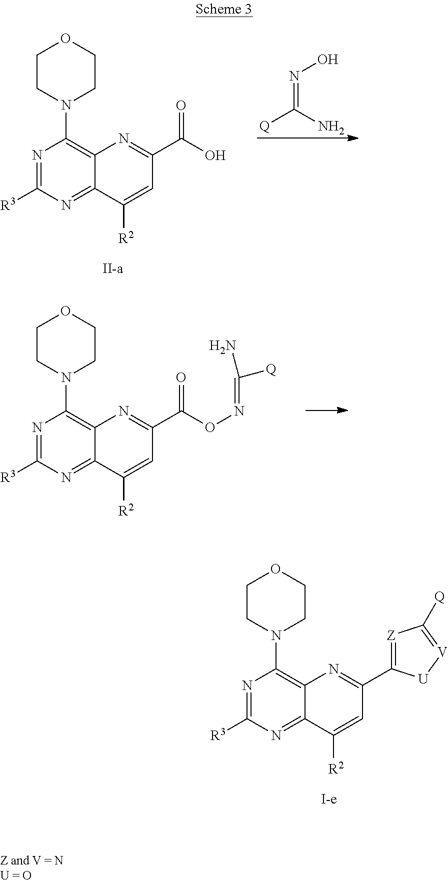

Preferrably, the method can be used for preparing the following compounds of Formula (I-e) selected below:
2-Imidazol-1-yl-6-(3-methyl-[1,2,4]oxadiazol-5-yl)-4-morpholin-4-yl-pyrido[3,2-d]pyrimidine
2-Imidazol-1-yl-6-(3-methoxymethyl-[1,2,4]oxadiazol-5-yl)-4-morpholin-4-yl-pyrido[3,2-d]pyrimidine
8-Chloro-2-imidazol-1-yl-6-(3-methyl-[1,2,4]oxadiazol-5-yl)-4-morpholin-4-yl-pyrido[3,2-d]pyrimidine
6-(3-Methyl-1,2,4-oxadiazol-5-yl)-4-morpholin-4-yl-2-(1H-pyrazol-1-yl)pyrido[3,2-d]pyrimidine
6-[3-(Methoxymethyl)-1,2,4-oxadiazol-5-yl]-4-morpholin-4-yl-2-(1H-pyrazol-1-yl)pyrido[3,2-d]pyrimidine

Compounds of formula (I-e) where U is N, V is N and Z is O can be prepared from compounds of Formula (II-a) where R² and R³ are as above defined, in a 2-step protocol as outlined in Scheme 4. The first step consists in the coupling of a carboxylic acid of formula (II-a) with an acyl hydrazide of Formula Q-C(O)—NHNH₂. General protocols for such coupling are given below in the examples, using conditions and methods well known to those skilled in the art to prepare an N,N′-bisacylated hydrazine from a carboxylic acid and an acetyl hydrazide, with standard coupling agents, such as but not limited to CDI, EDC, HATU, TBTU, in the presence or absence of bases such as TEA, DIEA, NMM in a suitable solvent such as DCM, ACN, THF or DMF, at a temperature rising from about 20° C. to about 50° C., preferably at RT, for a few hours, e.g. one hour to 24 h. Alternatively, a carboxylic acid derivative (e.g. acyl chloride) may be coupled with the amidoxime, using conditions and methods well known to those skilled in the art, in the presence of bases such as TEA, DIEA, NMM in a suitable solvent such as DCM, THF or DMF, at a temperature rising from about 20° C. to about 50° C., preferably at RT, for a few hours, e.g. one hour to 24 h (Scheme 4). The second step consists of the cyclization and dehydration of the N,N′-bisacylated hydrazine to form the oxadiazole (I-e). Conditions are given below in the examples, using methods well known to those skilled in the art to prepare oxadiazole, such as heating at temperature rising from RT to about 150° C., typically 100° C., using possibly a microwave oven, for a few hours, in the presence of POCl₃, or in a solvent such as sulphuric acid.

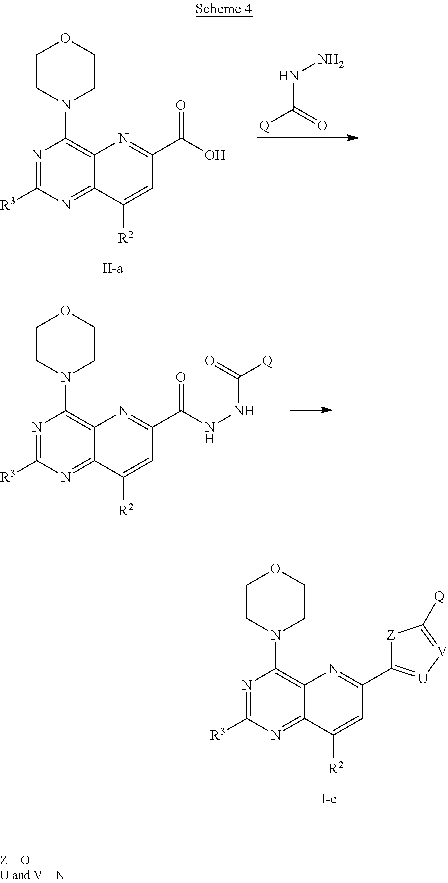

Preferrably, the method can be used for preparing the following compounds of Formula (I-e) selected below:
2-Imidazol-1-yl-6-(5-methyl-[1,3,4]oxadiazol-2-yl)-4-morpholin-4-yl-pyrido[3,2-d]pyrimidine

Compounds of Formula (I) wherein R¹ and R³ are as above defined and R² is —CH₂NH₂₋ₘAₘ can be prepared from the compounds of Formula E, wherein R¹ and R³ are as above defined, by reaction with a trifluoroborate salt of formula AₘNH₂₋ₘCH₂BF₃— (J. Org. Chem., 2008, 73, 2052-2057) in the presence of a palladium source such as palladium acetate, of a ligand such as X-Phos or S-Phos and in the presence or the absence of a base such as potassium carbonate, in a solvent such as THF, dioxane, water or a mixture thereof, at a temperature ranging from RT to 150° C., preferably 120° C. for a few hours (Scheme 5).

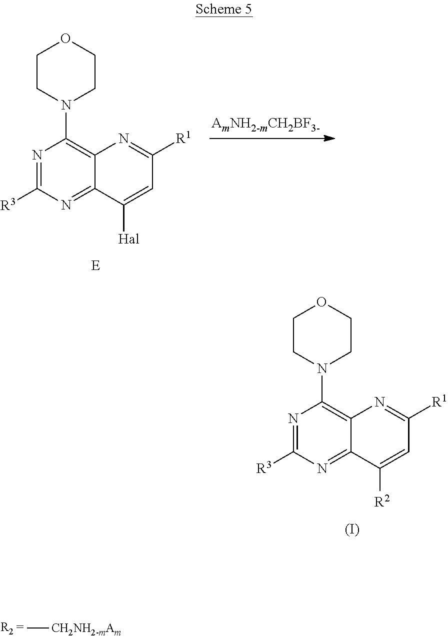

Preferrably, the method can be used for preparing the following compounds of Formula (I) selected below:
2-Imidazol-1-yl-6-methoxymethyl-4-morpholin-4-yl-8-morpholin-4-ylmethyl-pyrido[3,2-d]pyrimidine

Compounds of Formula (I-a) where X is CH₂, B is O and W is H can be prepared from the corresponding esters of Formula B, wherein R² and R³ are as above defined, by reaction with a reducing agent. Such reducing agent is preferrably LiBH₄, LAH, DIBAL, in a suitable solvent. Said solvent is preferably a lower alcohol, an ethereal solvent such as Et₂O, THF or dioxane, or mixture thereof (Scheme 6).

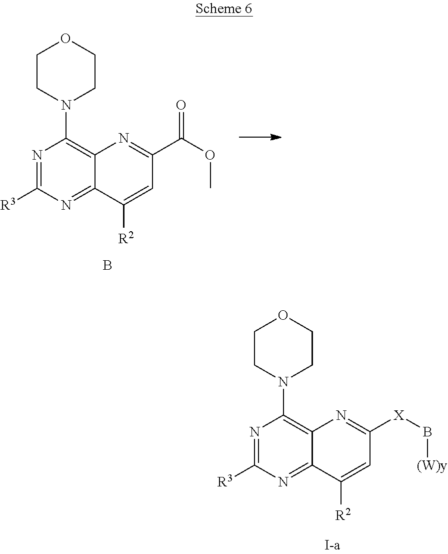

Preferrably, the method can be used for preparing the following compounds of Formula (I-a) selected below:
[8-(Methylthio)-4-morpholin-4-yl-2-(3-thienyl)pyrido[3,2-d]pyrimidin-6-yl]methanol
[2-(1H-Imidazol-1-yl)-4-morpholin-4-ylpyrido[3,2-d]pyrimidin-6-yl]methanol
[2-(1H-Imidazol-1-yl)-4,8-dimorpholin-4-ylpyrido[3,2-d]pyrimidin-6-yl]methanol

Compounds of Formula (I-a) where X is CO, B is N and W is (C1-C8)alkyl can be prepared from the esters C, wherein R² and R³ are as above defined, either directly by reaction with an amine with heating, or via the formation of the corresponding acids of Formula (II-a) and subsequent coupling with an amine of Formula N(w)_{y} wherein W and y are as above defined. In the case of the formation of the acid (II-a), compounds of Formula (I-a) can be obtained using usual conditions for the formation of an amide starting from a carboxylic acid and an amine by using a coupling agent such as HOBt, EDC, HATU or via the formation of an acid chloride or an activated ester. The carboxylic acids of Formula (II-a) can be obtained by hydrolysis of the esters C using reagents such as, but not limited to, LiOH, NaOH or KOH in solvents such water, a lower alcohol, THF, dioxane, or mixture thereof (Scheme 7).

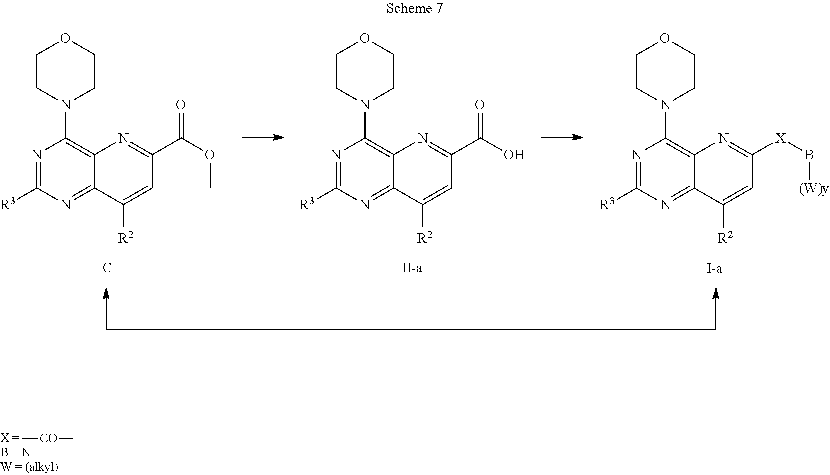

Preferrably, the method can be used for preparing the following compounds of Formula (I-a) selected below:
2-(1H-Imidazol-1-yl)-N,N-dimethyl-4-morpholin-4-ylpyrido[3,2-d]pyrimidine-6-carboxamide
2-(1H-Imidazol-1-yl)-N-methyl-4-morpholin-4-ylpyrido[3,2-d]pyrimidine-6-carboxamide
2-(1H-Imidazol-1-yl)-4-morpholin-4-ylpyrido[3,2-c]pyrimidine-6-carboxamide
2-(3-Hydroxymethyl-phenyl)-8-thiomethyl-4-morpholin-4-yl-pyrido[3,2-d]pyrimidine-6-carboxylic acid dimethylamide
2-(1H-Indazol-4-yl)-8-thiomethyl-4-morpholin-4-yl-pyrido[3,2-d]pyrimidine-6-carboxylic acid dimethylamide

Compounds of Formula (IV-a) wherein R³ is defined as above and W is N(H)₂₋ₘ(A)ₘ, (B(W)_{y} of the Formula (I-a) being equal to N(H)₂₋ₘ(A)ₘ) can be prepared from compounds of Formula D, wherein Hal is F, Cl, Br or I, preferably Cl or Br, by reaction with an amine with heating, either neat, or in the presence or the absence of a base such as, but not limited to, TEA or DIEA, in a solvent such as THF, dioxane, DMA, DMF, ACN or mixture thereof (Scheme 8)

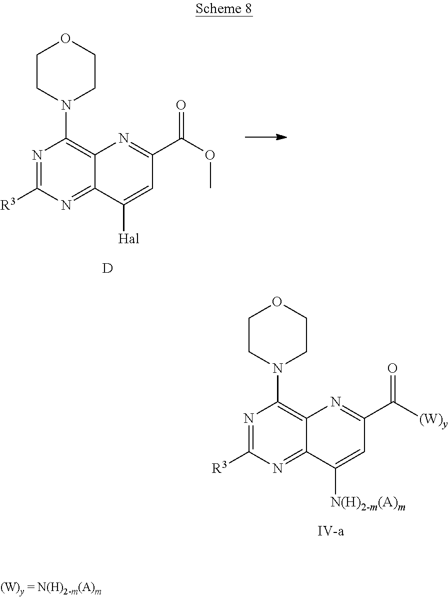

Preferrably, the method can be used for preparing the following compounds of Formula (I-a) selected below:
N-[2-(Dimethylamino)ethyl]-8-{[2-(dimethylamino)ethyl]amino}-2-(1H-imidazol-1-yl)-4-morpholin-4-ylpyrido[3,2-d]pyrimidine-6-carboxamide

Compounds of Formula (I) wherein R¹ and R³ are as above defined and R² is H can be prepared from the compounds of Formula E, wherein R¹ and R³ are as above defined and R² is Hal or SA, by reaction with a reducing agent. Such reducing agent can be Raney Nickel with or without hydrogen. The reaction can also be performed in the presence of hydrogen, which can be generated or not from a salt such as ammonium formate, and a metal catalyst such as Pd/C (Scheme 9).

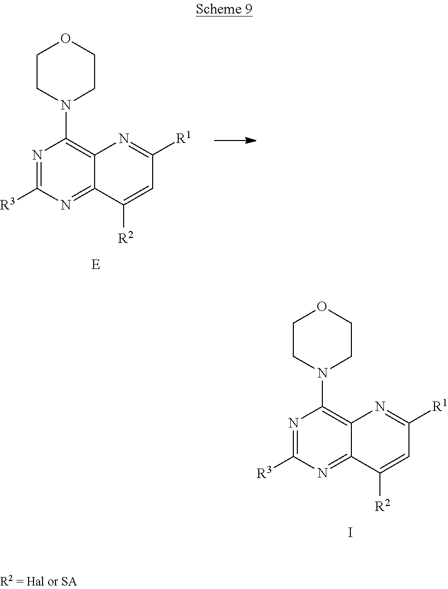

Preferrably, the method can be used for preparing the following compounds of Formula (I) selected below:
Methyl 2-(1H-imidazol-1-yl)-4-morpholin-4-ylpyrido[3,2-d]pyrimidine-6-carboxylate
Methyl 4-morpholin-4-yl-2-phenylpyrido[3,2-d]pyrimidine-6-carboxylate

Compounds of Formula (I) wherein R¹ is as defined above, and R² and R³ are equal and are Het linked through a C—N bond, can be prepared from compounds of Formula F wherein Hal is F, Cl, Br or I, preferably Cl or Br, by reaction with Het-H in the presence of a base such as Cs₂CO₃, K₂CO₃ or NaH, in a solvent such as THF, dioxane, DMF, DMA or a mixture thereof (Scheme 10).

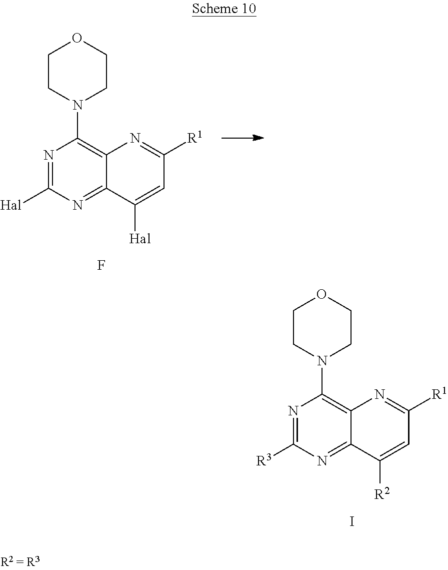

Preferrably, the method can be used for preparing the following compounds of Formula (I) selected below:
Methyl 4-morpholin-4-yl-2,8-di-1H-pyrrol-1-ylpyrido[3,2-d]pyrimidine-6-carboxylate

Compounds of Formula (V-a) wherein R¹ and R³ are as above defined can be prepared from the compounds of Formula (I) wherein R¹ and R³ are as above defined and R² is Hal, preferably Cl or Br, or SO₂A, by reaction with an amine of Formula HN(H)₂₋ₘ(A)ₘ (or a salt thereof), in the presence or the absence of a base such as TEA or DIEA in a solvent such as THF, dioxane, DMF, DMA or DMSO (Scheme 11).

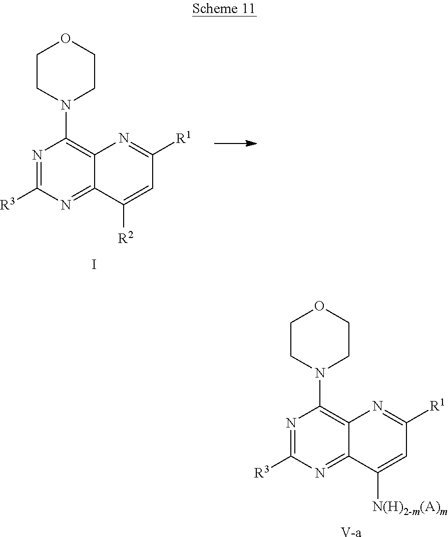

Preferrably, the method can be used for preparing the following compounds of Formula (I) selected below:
Methyl 2-(1H-imidazol-1-yl)-4,8-dimorpholin-4-ylpyrido[3,2-d]pyrimidine-6-carboxylate
Methyl 8-(dimethylamino)-2-(1H-imidazol-1-yl)-4-morpholin-4-ylpyrido[3,2-d]pyrimidine-6-carboxylate
Methyl 2-(1H-Imidazol-1-yl)-8-[(2-methoxyethyl)(methyl)amino]-4-morpholin-4-ylpyrido[3,2-d]pyrimidine-6-carboxylate
Methyl 2-(1H-imidazol-1-yl)-8-(4-methylpiperazin-1-yl)-4-morpholin-4-ylpyrido[3,2-d]pyrimidine-6-carboxylate
Methyl 2-(1H-imidazol-1-yl)-8-[(2-methoxyethyl)amino]-4-morpholin-4-ylpyrido[3,2-d]pyrimidine-6-carboxylate
2-Imidazol-1-yl-6-(3-methyl-[1,2,4]oxadiazol-5-yl)-4-morpholin-4-yl-8-(2-methoxy-ethyl)methylamino)-pyrido[3,2-d]pyrimidine
8-Dimethylamino-2-Imidazol-1-yl-6-(3-methyl-[1,2,4]oxadiazol-5-yl)-4-morpholin-4-yl-pyrido[3,2-d]pyrimidine
2-Imidazol-1-yl-6-(5-methyl-[1,3,4]oxadiazol-2-yl)-4-morpholin-4-yl-8-[(2-methoxy-ethyl)methylamino]-pyrido[3,2-d]pyrimidine
2-Imidazol-1-yl-6-(5-methyl-[1,3,4]oxadiazol-2-yl)-4-morpholin-4-yl-8-dimethyl amino-pyrido[3,2-d]pyrimidine
8-Dimethylamino-2-Imidazol-1-yl-6-methoxymethyl-4-morpholin-4-yl-pyrido[3,2-d]pyrimidine
8-[(2-Methoxy-ethyl)-methylamino]-6-(3-methyl-[1,2,4]oxadiazol-5-yl)-4-morpholin-4-yl-2-pyrazol-1-yl-pyrido[3,2-d]pyrimidine
8-Dimethylamino-[6-(3-methyl-[1,2,4]oxadiazol-5-yl)-4-morpholin-4-yl-2-pyrazol-1-yl-pyrido[3,2-d]pyrimidine

Compounds of Formula (VI-a) and (VI-b) wherein R¹ and R³ are as above defined can be prepared from the compounds of Formula H wherein R¹ and R³ are as above defined, by reaction with an oxidant such as m-CPBA in a solvent such as DCM or DCE, in the presence or the absence of a base such as sodium hydrogenocarbonate (Scheme 12)

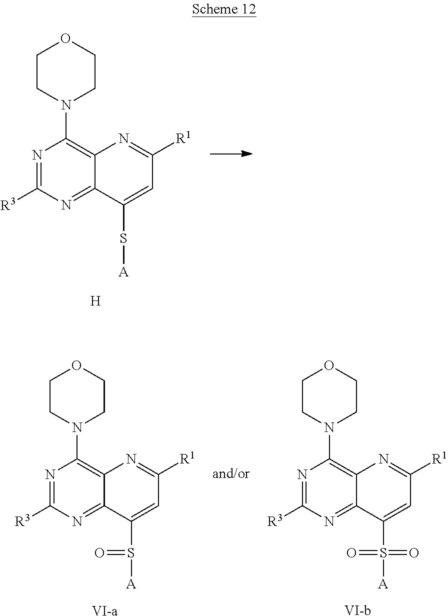

Preferrably, the method can be used for preparing the following compounds of Formula (I) selected below:
Methyl 2-chloro-8-(methylsulfonyl)-4-morpholin-4-ylpyrido[3,2-d]pyrimidine-6-carboxylate

Compounds of Formula H wherein R¹ and R³ are as above defined, can be prepared from the compounds of Formula J wherein Hal is F, Cl, Br or I, preferably Cl or Br, by reaction with a thiol or a salt thereof, in the presence or the absence of a base such as TEA or DIEA, in a solvent such as THF, dioxane, ACN, DMF, DMA (Scheme 13).

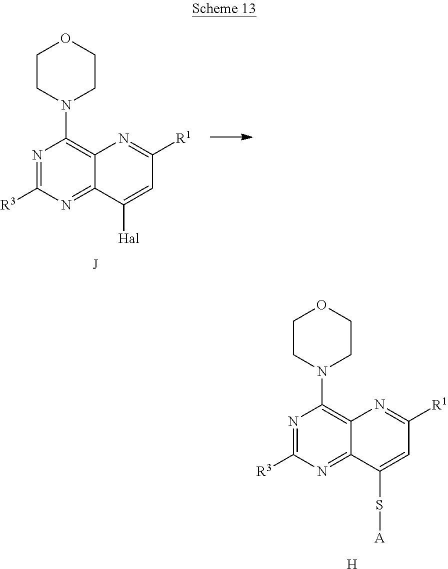

Preferrably, the method can be used for preparing the following compounds of Formula (I) selected below:
Methyl 2-chloro-8-methylsulfanyl-4-morpholin-4-yl-pyrido[3,2-d]pyrimidine-6-carboxylate
2-Imidazol-1-yl-6-(3-methyl-[1,2,4]oxadiazol-5-yl)-8-thiomethyl-4-morpholin-4-yl-pyrido[3,2-d]pyrimidine
2-Imidazol-1-yl-6-(5-methyl-[1,3,4]oxadiazol-2-yl)-4-morpholin-4-yl-8-thiomethyl-pyrido[3,2-d]pyrimidine
2-Imidazol-1-yl-6-methoxymethyl-8-thiomethyl-4-morpholin-4-yl-pyrido[3,2-d]pyrimidine
6-(3-Methyl-[1,2,4]oxadiazol-5-yl)-8-thiomethyl-4-morpholin-4-yl-2-pyrazol-1-yl-pyrido[3,2-d]pyrimidine

Compounds of Formula (I) where R¹ is as above defined, R² is SA or H, R³ is (C1-C8)alkyl, Ar or Het linked through a C—C bond, can be prepared from compounds of Formula K wherein R¹ is as above defined, R² is SA or H, via a cross coupling reaction with a boronic ester, a boronic acid or a trialkylstannane in the presence of a palladium source such as Pd(PPh₃)₄, as Pd(PPh₃)₂Cl₂, Pd₂(dba)₃, Pd(OAc)₂ or PdCl₂(ACN)₂, in the presence of a base such as TEA, DIEA, Cs₂CO₃, K₂CO₃ in solvent such as THF, dioxane, toluene, EtOH, DMF or DMA, or a mixture thereof (Scheme 14).

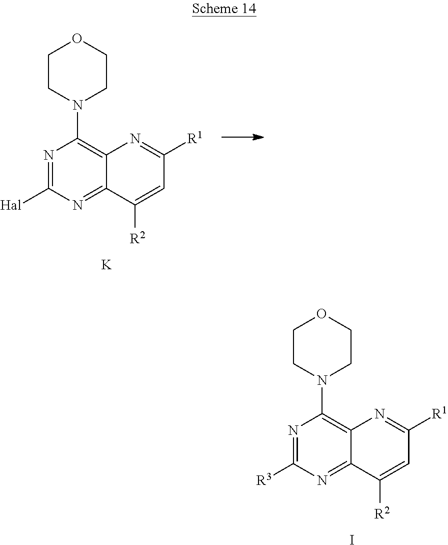

Preferrably, the method can be used for preparing the following compounds of Formula (I) selected below:
Methyl 8-(methylthio)-4-morpholin-4-yl-2-(3-thienyl)pyrido[3,2-d]pyrimidine-6-carboxylate
Methyl 8-(methylthio)-4-morpholin-4-yl-2-phenylpyrido[3,2-d]pyrimidine-6-carboxylate
Methyl 2-(1H-indol-4-yl)-8-(methylthio)-4-morpholin-4-ylpyrido[3,2-d]pyrimidine-6-carboxylate
Methyl 2-(1-methyl-1H-pyrazol-4-yl)-8-(methylthio)-4-morpholin-4-ylpyrido[3,2-d]pyrimidine-6-carboxylate
Methyl 8-(methylthio)-4-morpholin-4-yl-2-(1H-pyrazol-4-yl)pyrido[3,2-d]pyrimidine-6-carboxylate
Methyl 8-(methylthio)-4-morpholin-4-yl-2-pyridin-4-ylpyrido[3,2-d]pyrimidine-6-carboxylate
Methyl 2-methyl-8-(methylthio)-4-morpholin-4-ylpyrido[3,2-d]pyrimidine-6-carboxylate
6-Methyl-4-morpholin-4-yl-2-phenylpyrido[3,2-d]pyrimidine
Methyl 2-(4-methoxyphenyl)-8-(methylthio)-4-morpholin-4-ylpyrido[3,2-d]pyrimidine-6-carboxylate

Compounds of Formula (I) wherein R¹ and R² are as above defined and R³ is Het linked through a C—N bond can be prepared from compounds of Formula K wherein Hal is preferably Cl or Br, by reaction with Het-H in the presence of a base such as Cs₂CO₃, K₂CO₃ or NaH, in a solvent such as THF, dioxane, DMF, DMA or a mixture thereof (Scheme 15).

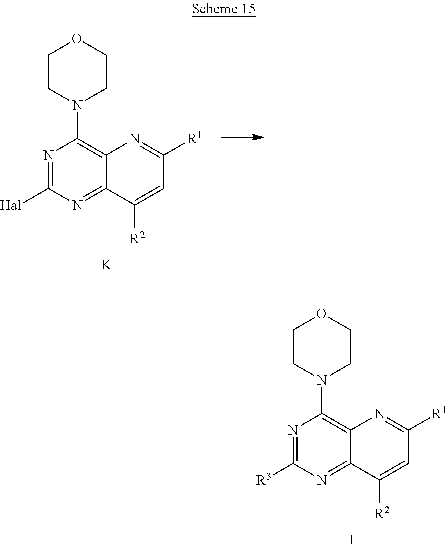

Preferrably, the method can be used for preparing the following compounds of Formula (I) selected below:
Methyl 8-chloro-2-(1H-imidazol-1-yl)-4-morpholin-4-ylpyrido[3,2-d]pyrimidine-6-carboxylate
Methyl 8-chloro-4-morpholin-4-yl-2-(1H-pyrazol-1-yl)pyrido[3,2-d]pyrimidine-6-carboxylate
Methyl 2-(1H-imidazol-1-yl)-8-(methylthio)-4-morpholin-4-ylpyrido[3,2-d]pyrimidine-6-carboxylate
2-(1H-Imidazol-1-yl)-6-methyl-4-morpholin-4-ylpyrido[3,2-d]pyrimidine
2-(1H-Imidazol-1-yl)-4-morpholin-4-ylpyrido[3,2-d]pyrimidine
Methyl 2-(1H-imidazol-1-yl)-8-(methylsulfonyl)-4-morpholin-4-ylpyrido[3,2-d]pyrimidine-6-carboxylate

Compounds of Formula (I) wherein R¹ is as above defined and R³ is Hal or H can be prepared from compounds of Formula (III-a), wherein R³ is Hal or H, by reaction with morpholine, in the presence or the absence of a base such as TEA or DIEA. The reaction is preferrably performed in a solvent such as ACN, THF, DCM, dioxane, DMF or DMA, or a mixture thereof (Scheme 16).

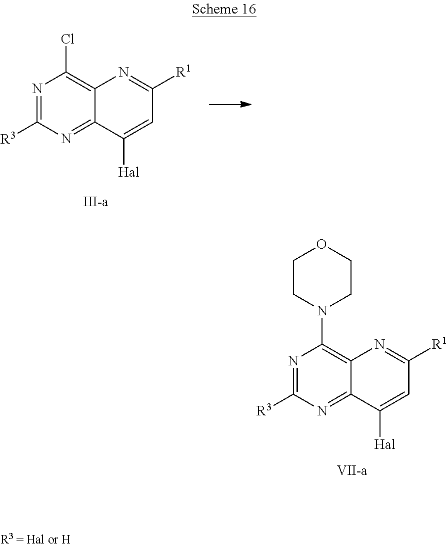

Preferrably, the method can be used for preparing the following compounds of Formula (I) selected below:
Methyl 2,8-dichloro-4-morpholin-4-yl-pyrido[3,2-c]pyrimidine-6-carboxylate
2-Chloro-6-methyl-4-morpholin-4-ylpyrido[3,2-d]pyrimidine
2-Chloro-4-morpholin-4-ylpyrido[3,2-d]pyrimidine

Compound of Formula (VIII-a) wherein R¹ is CO₂CH₃, R² and R³ are Cl can be prepared from 5-aminouracil in three steps as described in J. Org. Chem. 1979, 44, 435-440. The first step consists in a 1,4 addition of 5-aminouracil on dimethyl acetylenedicarboxylate. Heating of the obtained intermediate affords the corresponding cyclized product which affords compound of Formula (VIIIa) where R¹ is CO₂CH₃, for instance, by reaction with POCl₃ in the presence of N,N-diethylaniline (Scheme 17).

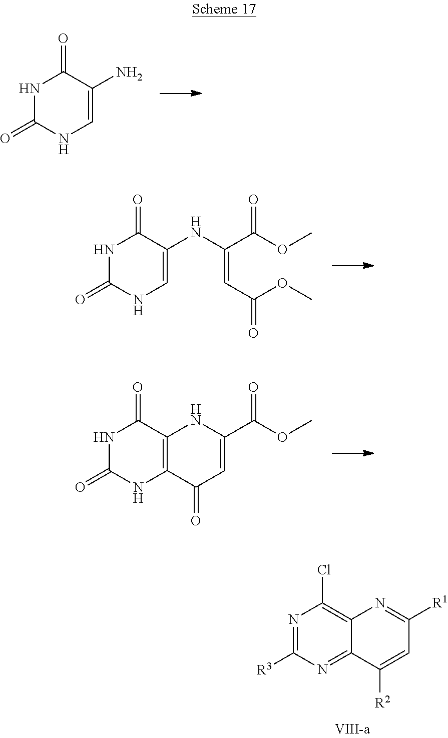

Compound of Formula (VIII-a) wherein R³ is Cl, R¹ and R² are H can be prepared from 2,3-pyridinecarboxylic anhydride as described in Synlett. 2006, 1938-1942. The first step consists the opening of the anhydride moiety with MeOH. The acid functionality of the intermediate formed can then be transformed into an isocyanate, which can be further reacted with 4-methoxybenzylamine and cyclized to give 3-(4-methoxybenzyl pyrido[3,2-d]pyrimidine-2,4(1H,3H)-dione. This intermediate can then be deprotected in the presence of a Lewis or Bronsted acid and further reacted with POCl₃ in the presence of PCl₅ (Scheme 18).

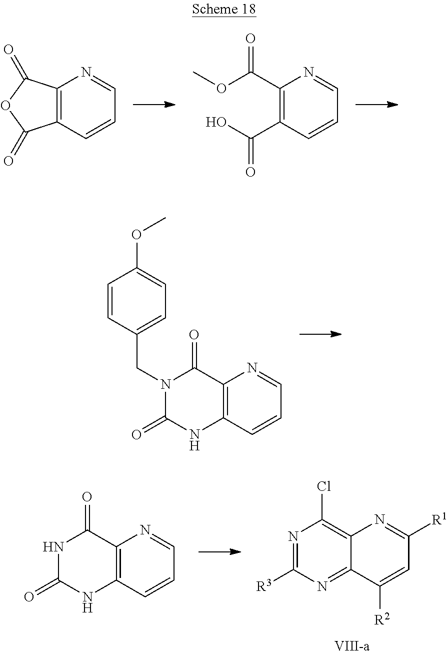

Compound of Formula (VIII-a) wherein R³ is Cl, R¹ is CH₃ and R² is H, can be prepared from 5-aminouracil in 2 steps as outlined in scheme 19. The first step consists in the condensation of 5-aminouracil with crotonaldehyde to give 6-methylpyrido[3,2-d]pyrimidine-2,4(1H,3H)-dione under acidic conditions. This intermediate is then reacted with POCl₃ in the presence of N,N-diethylaniline to afford compound of Formula (VIII-a) wherein R³ is Cl, R¹ is CH₃ and R² is H

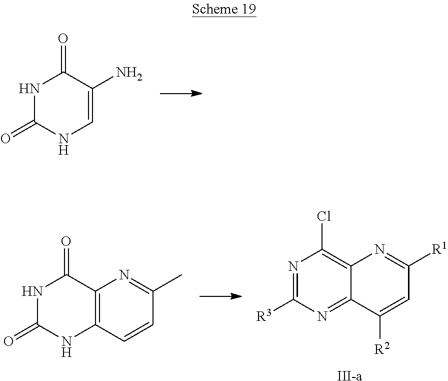

In a preferred embodiment the process of making compounds of Formula (I) includes the transformation of the hydroxy group of compounds of Formula A into a leaving group.

In another preferred embodiment, compounds of Formula (I) wherein R¹ is CO₂(C1-C8)alkyl or H and R² is Hal or H, may be obtained by reacting the intermediate M wherein R¹ is CO₂(C1-C8)alkyl or H and R² is Hal or H, with morpholine.

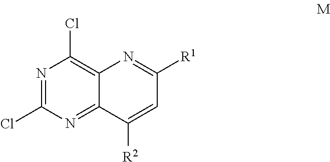

The compounds of the formula (I) and related formulae and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), under reaction conditions which are known and suitable for the said reactions. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3^{rd} Edition 1999.

Use can also be made here of variants which are known per se, but are not mentioned here in greater detail.

If desired, the starting materials can also be formed in situ so that they are not isolated from the reaction mixture, but instead are immediately converted further into the compounds of the formula (I).

The starting compounds for the preparation of compounds of formula (I) and related formulae are generally known. If they are novel, they can, however, be prepared by methods known per se.

The reactions are preferably carried out in an inert solvent.

Examples of suitable inert solvents are hydrocarbons, such as hexane, petroleum ether, benzene, toluene or xylene; chlorinated hydrocarbons, such as trichloroethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether or ethylene glycol dimethyl ether (diglyme); ketones, such as acetone or butanone; amides, such as acetamide, dimethylacetamide or dimethyl-formamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, or mixtures of the said solvents.

Accordingly, the invention relates, in particular, to the use of formula (I) and related formulae as defined above, as a medicament.

Accordingly, the invention relates, in particular, to the use of compounds of the formula (I) and related formulae as defined above, for the preparation of pharmaceutical formulations for the prevention and/or the treatment of multiple sclerosis, cancers and related disorders.

The said compounds of the formula (I) and related formulae can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula (I) are for the most part prepared by conventional methods. If the compound of the formula I and related formulae contains an acidic center, such as a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example sodium- or potassium methoxide and sodium or potassiumpropoxide, alkalihydrides, such as sodium- or potassiumhydride; and various organic bases, such as piperidine, diethanolamine and N-methyl-glutamine, benzathine, choline, diethanolamine, ethylenediamine, meglumine, benethamine, diethylamine, piperazine and tromethamine. The aluminium salts of the compounds of the formula (I) and related formulae are likewise included. In the case of certain compounds of the formula I and related formulae, which contain a basic center, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoaryl-sulfonates, such as ethanesulfonate, toluenesulfonate and benzene-sulfonate, and other organic acids and corresponding salts thereof, such as acetate, trifluoro-acetate, tartrate, maleate, succinate, citrate, benzoate, salicylate, ascorbate and the like. Accordingly, pharmaceutically acceptable acid-addition salts of the compounds of the formula I and related formulae include the following: acetate, adipate, alginate, arginate, aspartate, benzoate, benzene-sulfonate (besylate), bisulfate, bisulfite, bromide, butyrate, camphorate, camphor-sulfonate, caprylate, chloride, chlorobenzoate, citrate, cyclo-pentane-propionate, digluconate, dihydrogen-phosphate, dinitrobenzoate, dodecyl-sulfate, ethanesulfonate, fumarate, galacterate (from mucic acid), galacturonate, glucoheptanoate, gluco-nate, glutamate, glycerophosphate, hemi-succinate, hemisulfate, heptanoate, hexanoate, hippurate, hydro-chloride, hydrobromide, hydroiodide, 2-hydroxy-ethane-sulfonate, iodide, isethionate, isobutyrate, lactate, lactobionate, malate, maleate, malonate, mandelate, metaphosphate, methanesulfonate, methylbenzoate, mono-hydrogen-phosphate, 2-naphthalenesulfonate, nicotinate, nitrate, oxalate, oleate, palmo-ate, pectinate, persulfate, phenylacetate, 3-phenylpropionate, phosphate, phosphonate, phthalate, but this does not represent a restriction. Both types of salts may be formed or interconverted preferably using ion-exchange resin techniques.

Furthermore, the base salts of the compounds of the formula (I) and related formulae include aluminium, ammonium, calcium, copper, iron(III), iron(II), lithium, magne-sium, manganese(III), manganese(II), potassium, sodium and zink salts, but this is not intended to represent a restriction. Of the above-mentioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger resins, for example arginine, betaine, caffeine, chloroprocaine, choline, N,N′-dibenzyl-ethylen-ediamine (benzathine), dicyclohexylamine, diethanol-amine, diethyl-amine, 2-diethyl-amino-ethanol, 2-dimethyl-amino-ethanol, ethanolamine, ethylenediamine, N-ethylmorpholine, N-ethyl-piperidine, glucamine, glucosamine, histidine, hydrabamine, isopropyl-amine, lido-caine, lysine, meglumine (N-methyl-D-glucamine), morpholine, piperazine, piperidine, polyamine resins, procaine, purines, theobromine, triethanol-amine, triethylamine, trimethylamine, tripropyl-amine and tris(hydroxy-methyl)-methylamine (tromethamine), but this is not intended to represent a restriction.

Compounds of the formula (I) and related formulae of the present invention which contain basic nitrogen-containing groups can be quaternised using agents such as (C₁-C₄)-alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, do-decyl, lauryl, myristyl and stearyl chloride, bromide and iodide; and aryl-(C₁-C₄)alkyl halides, for example benzyl chloride and phenethyl bromide. Both water- and oil-soluble compounds of the formula I can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, me-glumine, nitrate, oleate, phosphonate, pivalate, sodium phosphate, stea-rate, sulfate, sulfosalicylate, tartrate, thiomalate, tosylate and tro-meth-amine, but this is not intended to represent a restriction.

The acid-addition salts of basic compounds of the formula (I) and related formulae are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts other-wise correspond to the respective free base forms thereof.

As mentioned, the pharmaceutically acceptable base-addition salts of the compounds of the formula (I) are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanol-amine, ethylenediamine, N-methyl-D-glucamine and procaine.

The base-addition salts of acidic compounds of the formula I and related formulae are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts other-wise correspond to the respective free acid forms thereof.

If a compound of the formula (I) and related formulae contains more than one group which is capable of forming pharmaceutically acceptable salts of this type, the formula (I) also encompasses multiple salts. Typical multiple salt forms include, for example, bitartrate, diacetate, difumarate, dimeglumine, di-phosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula (I) and related formulae in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

The term “leaving group” or “leaving groups” denotes an atom or a group of atoms easily cleaved, hydrolysed or substituted with a reagent. Preferred leaving groups are halogens, alkylsulfonates, arylsulfonates, alcoholates or activated esters.

The term “reducing agent” denotes a reagent able to donate electrons. Preferred reducing agents are Boranes, Catecholborane, Copper hydride, Copper (low valent), Chromium (low valent), Decaborane, DIBAL-H, Diborane, Diethyl 1,4-dihydro-2,6-dimethyl-3,5-pyridinedicarboxylate, Diisobutylaluminium hydride, Dimethylsulfide borane, DMSB, Fe, Formaldehyde, Formic acid, Hantzsch Ester, Hydrazine, Hydrogen, Indium (low valent), Iron, Isopropanol, LAH, Lithium, Lithium aluminum hydride, Lithium tetrahydridoaluminate, LiBH4, Magnesium, Manganese, 3-Mercaptopropionic acid, 3-MPA, Neodymium (low valent), Nickel, Nickel borohydride, Niobium (low valent), Phenylsilane, PMHS, Polymethylhydrosiloxane, Potassium, 2-Propanol, Red-Al, Rongalite, Samarium (low valent), Silanes, Sodium, Sodium bis(2-methoxyethoxy)aluminumhydride, Sodium borohydride, Sodium cyanoborohydride, Sodium dithionite, Sodium hydrosulfite, Sodium hydroxymethanesulfinate, Sodium tetrahydroborate, Sodium triacetoxyborohydride, Strontium, Tetramethyldisiloxane, Tin hydrides, Titanium (low valent), TMDSO, Tributylstannane, Tributyltin hydride, Trichlorosilane, Triphenylphosphine, Triphenylphosphite, Triethylsilane, Tris(trimethylsilyl)silane, TTMSS, Zinc.

The term “prodrug derivatives” or “prodrug” is taken to mean compounds of the formula (I) which have been modified with, for example, alkyl or acyl groups, sugars or oligopeptides and which are rapidly cleaved in the organism to form the active compounds.

These also include biodegradable polymer derivatives of the compounds according to the invention, as described, for example, in Int. J. Pharm. 115, 61-67 (1995).

Owing to their molecular structure, the compounds of the formula (I) and related formulae can be chiral and can accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the intermediates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitable N-protected amino acids (for example N-benzoylproline or N-benzenesulfonylproline), or the various optically active camphorsulfonic acids. Also advantageous is chromatographic enantiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or alcoholic solvent mixtures, such as, for example, hexane/isopropanol/acetonitrile, for example in the ratio 82:15:3.

The invention furthermore relates to the use of compounds of formula (I) and related formulae in combination with at least one further medicament active ingredient, preferably medicaments used in the treatment of multiple sclerosis such as cladribine or another co-agent, such as interferon, e.g. pegylated or non-pegylated interferons, preferably interferon beta and/or with compounds improving vascular function or in combination with immunomodulating agents for example Fingolimod; cyclosporins, rapamycins or ascomycins, or their immunosuppressive analogs, e.g. cyclosporin A, cyclosporin G, FK-506, ABT-281, ASM981, rapamycin, 40-O-(2-hydroxy)ethyl-rapamycin etc.; corticosteroids; cyclophosphamide; azathioprene; methotrexate; leflunomide; mizoribine; mycophenolic add; mycophenolate mofetil; 15-deoxyspergualine; diflucortolone valerate; difluprednate; Alclometasone dipropionate; amcinonide; amsacrine; asparaginase; azathioprine; basiliximab; beclometasone dipropionate; betamethasone; betamethasone acetate; betamethasone dipropionate; betamethasone phosphate sodique; betamethasone valerate; budesonide; captopril; chlormethine chlorhydrate; cladribine; clobetasol propionate; cortisone acetate; cortivazol; cyclophosphamide; cytarabine; daclizumab; dactinomycine; desonide; desoximetasone; dexamethasone; dexamethasone acetate; dexamethasone isonicotinate; dexamethasone metasulfobenzoate sodique; dexamethasone phosphate; dexamethasone tebutate; dichlorisone acetate; doxorubicine chlorhydrate; epirubicine chlorhydrate; fluclorolone acetonide; fludrocortisone acetate; fludroxycortide; flumetasone pivalate; flunisolide; fluocinolone acetonide; fluocinonide; fluocortolone; fluocortolone hexanoate; fluocortolone pivalate; fluorometholone; fluprednidene acetate; fluticasone propionate; gemcitabine chlorhydrate; halcinonide; hydrocortisone, hydrocortisone acetate, hydrocortisone butyrate, hydrocortisone hemisuccinate; melphalan; meprednisone; mercaptopurine; methylprednisolone; methylprednisolone acetate; methylprednisolone hemisuccinate; misoprostol; muromonab-cd3; mycophenolate mofetil; paramethasone acetate; prednazoline, prednisolone; prednisolone acetate; prednisolone caproate; prednisolone metasulfobenzoate sodique; prednisolone phosphate sodique; prednisone; prednylidene; rifampicine; rifampicine sodique; tacrolimus; thalidomide; thiotepa; tixocortol pivalate; triamcinolone; triamcinolone acetonide hemisuccinate; triamcinolone benetonide; triamcinolone diacetate; triamcinolone hexacetonide; immunosuppressive monoclonal antibodies, e.g., monoclonal antibodies to leukocyte receptors, e.g., MHC, CD2, CD3, CD4, CD7, CD25, CD28, B7, CD40, CD45 or CD58 or their ligands; or other immunomodulatory compounds, e.g. CTLA41g, or other adhesion molecule inhibitors, e.g. mAbs or low molecular weight inhibitors including Selectin antagonists and VLA-4 antagonists. A preferred composition is with Cyclosporin A, FK506, rapamycin or 40-(2-hydroxy)ethyl-rapamycin and Fingolimod. These further medicaments, such as interferon beta, may be administered concomitantly or sequentially, e.g. by subcutaneous, intramuscular or oral routes.

The invention furthermore relates to the use of compounds of formula I and related formulae in combination with at least one further medicament active ingredient, preferably medicaments used in the treatment of cancer wherein said antitumoral compounds are selected from those well known by the one skilled in the related art.

These compositions can be used as medicaments in human and veterinary medicine.

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medica-ment after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be formulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula (I) and related formulae and salts, solvates and physiologically functional derivatives thereof and the other active ingredients can also be administered in the form of liposome delivery systems, such as, for exam-ple, small unilamellar vesicles, large unilamellar vesicles and multilamellar vesicles. Liposomes can be formed from various phospholipids, such as, for example, cholesterol, stearylamine or phosphatidylcholines.

The compounds of the formula (I) and related formulae and the salts, solvates and physiologically functional derivatives thereof and the other active ingredients can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropyl-methacrylamidophenol, polyhydroxyethylaspartamido-phenol or polyethylene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, poly-orthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or sus-pended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insuf-flators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I and related formulae and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

The present invention furthermore relates to a method for treating a subject suffering from a PI3K related disorder, comprising administering to said subject an effective amount of a compound of formula I and related formulae. The present invention preferably relates to a method, wherein the PI3K associated disorder is an autoimmune disorder or condition associated with an overactive immune response or cancer. The present invention furthermore relates to a method of treating a subject suffering from an immunerogulatory abnomality, comprising administering to said subject a compound of formula (I) and related formulae in an amount that is effective for treating said immunoregulatory abnormality. The present invention preferably relates to a method wherein the immunoregulatory abnormality is an autoimmune or chronic inflammatory disease selected from the group consisting of: amyotrophic lateral sclerosis (ALS), systemic lupus erythematosus, chronic rheumatoid arthritis, type I diabetes mellitus, inflammatory bowel disease, biliary cirrhosis, uveitis, multiple sclerosis, Crohn's disease, ulcerative colitis, bullous pemphigoid, sarcoidosis, psoriasis, autoimmune myositis, Wegener's granulomatosis, ichthyosis, Graves ophthalmopathy and asthma. The present invention furthermore relates to a method wherein the immunoregulatory abnormality is bone marrow or organ transplant rejection or graft-versus-host disease. The present invention furthermore relates to a method wherein the immunoregulatory abnormality is selected from the group consisting of: transplantation of organs or tissue, graft-versus-host diseases brought about by transplantation, autoimmune syndromes including rheumatoid arthritis, systemic lupus erythematosus, Hashimoto's thyroiditis, multiple sclerosis, myasthenia gravis, type I diabetes, uveitis, posterior uveitis, allergic encephalomyelitis, glomerulonephritis, post-infectious autoimmune diseases including rheumatic fever and post-infectious glomerulonephritis, inflammatory and hyperproliferative skin diseases, psoriasis, atopic dermatitis, contact dermatitis, eczematous dermatitis, seborrhoeic dermatitis, lichen planus, pemphigus, bullous pemphigoid, epidermolysis bullosa, urticaria, angioedemas, vasculitis, erythema, cutaneous eosinophilia, lupus erythematosus, acne, alopecia greata, keratoconjunctivitis, vernal conjunctivitis, uveitis associated with Behcet's disease, keratitis, herpetic keratitis, conical cornea, dystrophia epithelialis corneae, corneal leukoma, ocular pemphigus, Mooren's ulcer, scleritis, Graves' opthalmopathy, Vogt-Koyanagi-Harada syndrome, sarcoidosis, pollen allergies, reversible obstructive airway disease, bronchial asthma, allergic asthma, intrinsic asthma, extrinsic asthma, dust asthma, chronic or inveterate asthma, late asthma and airway hyper-responsiveness, bronchitis, gastric ulcers, vascular damage caused by ischemic diseases and thrombosis, ischemic bowel diseases, inflammatory bowel diseases, necrotizing enterocolitis, intestinal lesions associated with thermal burns, coeliac diseases, proctitis, eosinophilic gastroenteritis, mastocytosis, Crohn's disease, ulcerative colitis, migraine, rhinitis, eczema, interstitial nephritis, Goodpasture's syndrome, hemolytic-uremic syndrome, diabetic nephropathy, multiple myositis, Guillain-Barre syndrome, Meniere's disease, polyneuritis, multiple neuritis, mononeuritis, radiculopathy, hyperthyroidism, Basedow's disease, pure red cell aplasia, aplastic anemia, hypoplastic anemia, idiopathic thrombocytopenic purpura, autoimmune hemolytic anemia, agranulocytosis, pernicious anemia, megaloblastic anemia, anerythroplasia, osteoporosis, sarcoidosis, fibroid lung, idiopathic interstitial pneumonia, dermatomyositis, leukoderma vulgaris, ichthyosis vulgaris, photoallergic sensitivity, cutaneous T cell lymphoma, chronic lymphocytic leukemia, arteriosclerosis, atherosclerosis, aortitis syndrome, polyarteritis nodosa, myocardosis, scleroderma, Wegener's granuloma, Sjogren's syndrome, adiposis, eosinophilic fascitis, lesions of gingiva, periodontium, alveolar bone, substantia ossea dentis, glomerulonephritis, male pattern alopecia or alopecia senilis by preventing epilation or providing hair germination and/or promoting hair generation and hair growth, muscular dystrophy, pyoderma and Sezary's syndrome, Addison's disease, ischemia-reperfusion injury of organs which occurs upon preservation, transplantation or ischemic disease, endotoxin-shock, pseudomembranous colitis, colitis caused by drug or radiation, ischemic acute renal insufficiency, chronic renal insufficiency, toxinosis caused by lung-oxygen or drugs, lung cancer, pulmonary emphysema, cataracta, siderosis, retinitis pigmentosa, senile macular degeneration, vitreal scarring, corneal alkali burn, dermatitis erythema multiforme, linear IgA ballous dermatitis and cement dermatitis, gingivitis, periodontitis, sepsis, pancreatitis, diseases caused by environmental pollution, aging, carcinogenesis, metastasis of carcinoma and hypobaropathy, disease caused by histamine or leukotriene-C₄ release, Behcet's disease, autoimmune hepatitis, primary biliary cirrhosis, sclerosing cholangitis, partial liver resection, acute liver necrosis, necrosis caused by toxin, viral hepatitis, shock, or anoxia, B-virus hepatitis, non-A/non-B hepatitis, cirrhosis, alcoholic cirrhosis, hepatic failure, fulminant hepatic failure, late-onset hepatic failure, “acute-on-chronic” liver failure, augmentation of chemotherapeutic effect, cytomegalovirus infection, HCMV infection, AIDS, cancer, senile dementia, parkison diseases, trauma, and chronic bacterial infection.

Preferred compounds of formula (I) and related formulae exhibit a IC₅₀ for the binding to PI3Kδ of less than about 5 μM, preferably less than about 1 μM and even more preferred less than about 0.010 μM.

Compounds according to formula formula (I) and related formulae may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimisation procedures.

In general, the synthesis pathways for any individual compound of formula (I) and related formulae will depend on the specific substitutents of each molecule and upon the ready availability of intermediates necessary; again such factors being appreciated by those of ordinary skill in the art.

Compounds of this invention can be isolated in association with solvent molecules by crystallization from evaporation of an appropriate solvent. The pharmaceutically acceptable acid addition salts of the compounds of formula (I) and related formulae which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of formula (I) and related formulae, which contain an acid center, with a suitable base. Both types of salts may be formed or interconverted using ion-exchange resin techniques.

The compounds of invention have been named according the standards used in the program “ACD/Name Batch” from Advanced Chemistry Development Inc., ACD/Labs (7.00 Release). Product version: 7.10, build: 15 Sep. 2003.

In the following the present invention shall be illustrated by means of some examples, which are not construed to be viewed as limiting the scope of the invention.

### Experimental Part

The commercially available starting materials used in the following experimental description were purchased from Sigma-Aldrich-Fluka unless otherwise reported. However, specific reagents were purchased from other suppliers: 1-(tert-butyldimethylsilyl)-1H-indol-4-ylboronic acid (Combiblocks), pyrazole-4-boronic acid (Boron-Mol), N-(2-methoxyethyl)methylamine (TCl).

The HPLC data provided in the examples described below were obtained as followed:

Method A: HPLC columns: Xbridge™ C8 column 50 mm×4.6 mm at a flow of 2 mL/min with 8 min gradient from 0.1% TFA in H₂O to 0.07% TFA in CH3CN. UV detection (maxplot).
Method B: HPLC columns: BDS C18 column 50 mm×4.6 mm at a flow of 0.8 mL/min with 8 minutes gradient from 0.1% TFA in H₂O to CH₃CN. UV detection (maxplot)
Method C: HPLC columns: Xbridge™ C8 column 30 mm×2.1 mm at a flow of 1 mL/min with 8 min gradient from 10 mM NH₄OAc in H₂O+5% CH3CN to CH₃CN. UV detection (maxplot).
Method D: HPLC columns: Atlantis C₁₈ column 50 mm×4.6 mm at a flow of 1.5 mL/min with 8 minutes gradient from 0.1% HCOOH in H₂O to MeOH. UV detection (maxplot).

The MS data provided in the examples described below were obtained as followed: LC/MS Waters ZMD (ESI).

The NMR data provided in the examples described below were obtained as followed: ¹H-NMR: Bruker DPX-300 MHz or 400 MHz

The microwave chemistry is performed on a single mode microwave reactor Emrys™ Optimiser from Personal Chemistry or on an Initiator from Biotage.

### EXAMPLES

### Intermediate 1

### Dimethyl (2E)-2-[(2,4-dioxo-1,2,3,4-tetrahydropyrimidin-5-yl)amino]but-2-enedioate

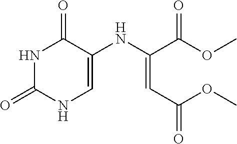

To a suspension of 5-aminouracil (275 g, 2.16 mol, 1 eq.) in dry methanol (5.5 L) was added dropwise dimethyl acetylene dicarboxylate (344 g, 2.42 mol, 1.1 eq.) at room temperature. After the end of the addition, the mixture was stirred at room temperature for 24 hours. The precipitate was filtered off, washed with methanol (500 mL) and dried under vacuum to afford the title compound (430 g, 74%) as a yellow solid.

¹H NMR (400 MHz, DMSO-d₆) δ 11.33 (br s, 1H), 10.82 (br s, 1H), 9.07 (s, 1H), 7.42 (s, 1H), 5.21 (s, 1H), 3.65 (s, 3H), 3.63 (s, 3H). LC/MS (Method B): RT 0.87 min (purity 96%). MS (ES−): 267.6.

### Intermediate 2

### Methyl 2,4,8-trioxo-1,2,3,4,5,8-hexahydropyrido[3,2-d]pyrimidine-6-carboxylate

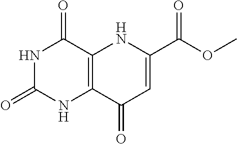

A suspension of intermediate 1 (50 g, 0.182 mol) in Dowtherm® A (1 L) was refluxed for 1 hour. The reaction mixture was cooled to room temperature and diluted with petroleum ether (2 L). The precipitate was filtered, washed with petroleum ether (1 L) and dried under vacuum. The crude product was slurred in DMF (200 mL) and the insoluble material was collected by filtration to afford the title compound as a brown solid. ¹H NMR (400 MHz, DMSO-d₆) δ 12.05 (br s, 1H), 11.57 (br s, 1H), 10.91 (br s, 1H), 7.57 (s, 1H), 3.86 (s, 3H). LC/MS (Method B): RT 0.77 min (purity 83%). MS (ES−): 235.8.

### Intermediate 3

### Methyl 2,4,8-trichloropyrido[3,2-d]pyrimidine-6-carboxylate

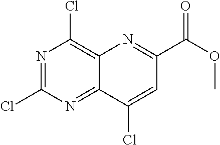

To a suspension of Intermediate 2 (10 g, 42.2 mmol) in phosphorous oxychloride (250 mL) was added dropwise N,N-diethylaniline (10 mL) at room temperature. After the end of the addition, the mixture was refluxed for 18 hours then concentrated in vacuo to ca. 50 mL. The residue was poured onto ice-water (1 L) and the solid was filtered off, washed with water and dried to afford the title compound (10 g, 81%) as a brown solid. ¹H NMR (400 MHz, DMSO-d₆) δ 8.46 (s, 1H), 3.93 (s, 3H). LC/MS (Method B): RT 1.52 min (purity 87%). MS (ES+): 293.1.

### Intermediate 4

### 6-Methylpyrido[3,2-d]pyrimidine-2,4(1H,3H)-dione

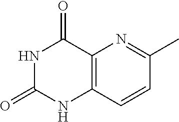

A mixture of 5-aminouracil (3 g; 23.6 mmol; 1 eq.) and crotonaldehyde (2.04 mL; 24.8 mmol; 1.05 eq.) in 20% HCl (20 mL) was stirred at 110° C. for 1 hour. The solution was evaporated in vacuo and the residue precipitated from EtOH to afford the title compound as a black solid. ¹H NMR (300 MHz, DMSO-d₆) δ 8.90-8.50 (m, 2H), 7.80 (d, 1H), 7.51 (d, 1H), 2.50 (s, 3H). MS (ES+): 178.1.

### Intermediate 5

### 2,4-Dichloro-6-methylpyrido[3,2-d]pyrimidine

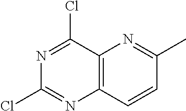

A mixture of Intermediate 4 (1 g; 5.64 mmol; 1 eq.), phosphorous oxychloride (17.3 mL; 113 mmol; 20 eq.) and N,N-diethylaniline (0.84 mL; 5.64 mmol; 1 eq.) was stirred at 130° C. for 4 hours then evaporated in vacuo. The residue was poured onto ice and extracted with DCM. The organic phase was dried over magnesium sulphate and concentrated in vacuo. Purification by column chromatography (petroleum ether/ethyl acetate, 90/10) afforded the title compound as a white solid.

¹H NMR (300 MHz, CDCl₃) δ 8.16 (d, J=8.7 Hz, 1H), 7.75 (d, J=8.7 Hz, 1H), 2.72 (s, 3H). HPLC (Method A): RT 3.21 min (purity 98%).

### Intermediate 6

### 2,4-Dichloro-6-methylpyrido[3,2-d]pyrimidine

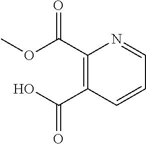

A solution of 2,3-pyridinedicarboxylic anhydride (30 g; 201 mmol; 1 eq.) in MeOH (300 mL) was refluxed for 48 hours then concentrated in vacuo. The crude was dissolved in hot ethyl acetate (200 mL) then allowed to return to room temperature and the precipitate filtered off. The solid was redissolved in hot ethyl acetate, cooled down to room temperature and filtered. The filtrate was concentrated in vacuo to afford the title compound. The first mother liquor was concentrated in vacuo and the residue recrystallized from ethyl acetate to afford the title compound. The two fractions were combined to afford the title compound as a white solid.

¹H NMR (300 MHz, DMSO-d₆) δ 13.80 (br s, 1H) 8.79 (dd, J=4.8, 1.6 Hz, 1H), 8.31 (dd, J=8.0, 1.6 Hz, 1H), 7.69 (dd, J=8.0, 4.8 Hz, 1H), 3.86 (s, 3H).

### Intermediate 7

### 3-(4-Methoxybenzyl)pyrido[3,2-d]pyrimidine-2,4(1H,3H)-dione

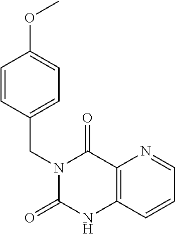

A solution of Intermediate 6 (10.3 g; 56.9 mmol; 1 eq.) and TEA (10.25 ml; 73.9 mmol; 1.3 eq.) in dry THF (206 mL) was cooled to −10° C. Ethyl chloroformate (8.19 ml; 85.3 mmol; 1.5 eq.) was added dropwise over 25 min) and the reaction mixture was stirred at this temperature for 1.5 hour. A solution of sodium azide (6.28 g; 96.7 mmol; 1.7 eq.) in water (103 mL) was then added in one portion. After 1.5 hour at 0° C., the resulting heterogenous mixture was filtered, the two liquid phases were separated and the aqueous phase was extracted with ethyl acetate (3×100 mL). The combined organic layer was dried over magnesium sulphate and concentrated in vacuo. The residue was taken up in toluene (62 mL) and stirred at refluxed for 2 hours. After cooling to room temperature, a solution of 4-methoxybenzylamine (7.36 ml; 56.9 mmol; 1 eq.) in pyridine (103 mL) was added and the reaction mixture was stirred at refluxed for 24 hours. After concentration in vacuo, the residue was washed with ethanol to afford the title compound as a white solid.

¹H NMR (300 MHz, DMSO-d₆) δ 11.54 (br s, 1H), 8.49 (dd, J=4.2, 1.5 Hz, 1H), 7.69-7.57 (m, 2H), 7.30 (d, J=8.7 Hz, 2H), 6.86 (d, J=8.7 Hz, 2H), 5.02 (s, 2H), 3.71 (s, 3H). HPLC (method A): RT 2.10 min (purity 91%).

### Intermediate 8

### Pyrido[3,2-d]pyrimidine-2,4(1H,3H)-dione

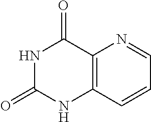

A solution of aluminum chloride (37.7 g; 282.4 mmol; 10 eq.) in anisole (80 mL) was added to a solution of Intermediate 7 (8 g; 28.2 mmol; 1 eq.) in anisole (80 mL) and the resulting mixture was stirred at room temperature for 16 hours. After cooling to 0° C., methanol (250 mL) was added, the mixture was stirred at this temperature for 10 minutes then concentrated in vacuo. The residue was suspended in ethyl acetate (250 mL), stirred for 15 minutes, and filtered. The solid was washed with ethyl acetate (2×250 mL) then suspended in water (400 mL). The mixture was stirred for 15 minutes, the precipitate filtered off, then washed with water and dried to afford the title compound as a white solid.

¹H NMR (300 MHz, DMSO-d₆) δ 11.49 (br s, 1H), 11.22 (br s, 1H), 8.45 (dd, J=4.2, 1.5 Hz, 1H), 7.66-7.54 (m, 2H).

### Intermediate 9

### 2,4-Dichloropyrido[3,2-d]pyrimidine

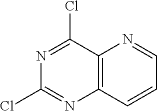

A mixture of Intermediate 8 (3.95 g; 24.2 mmol; 1 eq.), POCl₃ (39.5 mL) and PCl_{S} (20.2 g; 96.8 mmol; 4 eq.) was refluxed for 4 hours then concentrated in vacuo. The residue was taken up in DCM and carefully washed sat. aq. Na₂CO₃. The organic layer was dried over magnesium sulphate and concentrated in vacuo to afford the title compound as a beige solid.

¹H NMR (300 MHz, DMSO-d₆) δ 9.21 (dd, J=4.2, 1.5 Hz, 1H), 8.50 (dd, J=8.7, 1.5 Hz, 1H), 8.16 (dd, J=8.7, 4.26 Hz, 1H).

### Intermediate 10

### 2-Chloro-4-morpholin-4-ylpyrido[3,2-d]pyrimidine

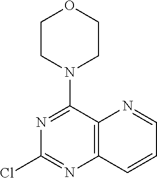

A solution of morpholine (0.22 mL; 2.5 mmol; 1 eq.) in THF (2 mL) was slowly added, at 0° C., to a solution of Intermediate 9 (500 mg; 2.5 mmol; 1 eq.) and N,N-diethylethanamine (0.38 ml; 2.75 mmol; 1.1 eq.) in ACN (5 mL) and THF (20 mL). The reaction mixture was stirred at this temperature for 3 hours then diluted with water. The aqueous layer was extracted with ethyl acetate, dried over magnesium sulphate and concentrated in vacuo. The residue was recrystallized from DCM/ACN to afford the title compound (550 mg, 88%) as a grey solid.

¹H NMR (300 MHz, CDCl₃) δ 8.71 (dd, J=4.2, 1.5 Hz, 1H), 8.09 (dd, J=8.5, 1.5 Hz, 1H), 7.64 (dd, J=8.5, 4.1 Hz, 1H), 5.10-4.20 (m, 4H), 3.94-3.86 (m, 4H). HPLC (Method A): RT 3.06 min (purity 96%).

### Intermediate 11

### 2-Imidazol-1-yl-4-morpholin-4-yl-pyrido[3,2-d]pyrimidine-6-carboxylic acid

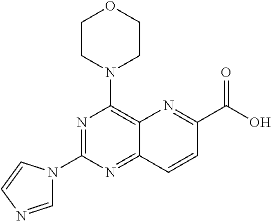

Lithium hydroxide (240 mg; 6 mmol) was added to a solution of Example 16 (1.6 g; 5 mmol) in a mixture of methanol/tetrahydrofurane/water (3:2:1, 100 mL) and the resulting mixture was stirred at 70° C. for 1 hour. The reaction mixture was concentrated in vacuo and the residue was neutralized at 0° C. with 20% aq. citric acid. The precipitate was filtered off, washed with cold water and dried under vacuum to afford the title compound (1.2 g, 78%) as a white solid.

¹H NMR (400 MHz, CDCl₃) δ 9.34 (s, 1H), 8.27 (d, J=8.7 Hz, 1H), 8.12 (s, 1H), 8.03 (d, J=8.7 Hz, 1H), 7.36 (s, 1H), 5.09 (m, 2H), 4.16 (m, 2H), 3.78 (t, J=4.9 Hz, 4H). HPLC (Method A): RT 1.96 min (purity 99%). MS (ES+): 327.0.

### Intermediate 12

### 8-Chloro-2-imidazol-1-yl-4-morpholin-4-yl-pyrido[3,2-d]pyrimidine-6-carboxylic acid

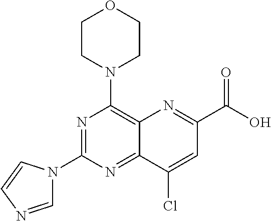

Lithium hydroxide (160 mg; 4 mmol) was added to a solution of Example 3 (1.0 g; 3 mmol) in a mixture of methanol/tetrahydrofurane/water (3:2:1, 50 mL) and the resulting mixture was stirred at 70° C. for 1 hour. The reaction mixture was concentrated in vacuo and the residue was neutralized at 0° C. with 20% aq. citric acid. The precipitate was filtered off, washed with cold water and dried under vacuum to afford the title compound as a white solid.

¹H NMR (400 MHz, DMSO-d₆) δ 8.66 (s, 1H), 8.38 (s, 1H), 7.98 (s, 1H), 7.12 (s, 1H), 5.12 (m, 2H), 4.32 (m, 2H), 3.80 (t, J=4.7 Hz, 4H). HPLC (Method C): RT 1.14 min (purity 99%). MS (ES−): 361.0.

### Intermediate 13

### 8-Chloro-2-imidazol-1-yl-6-(5-methyl-[1,3,4]oxadiazol-2-yl)-4-morpholin-4-yl-pyrido[3,2-d]pyrimidine

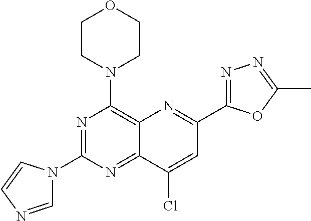

Oxalyl chloride (0.4 mL; 4.22 mmol) was added at 0° C. to a solution of Intermediate 12 (380 mg; 1.05 mmol) and DMF (2 drops) in DCM (3 mL) and the resulting mixture and stirred for 3 hours. After concentration in vacuo, the residue was taken up in DCM (2 mL) and added, at 0° C., to a solution of acetic hydrazide (94 mg; 1.26 mmol) and DIEA (272 mg; 2.11 mmol) in DCM (2 mL). The reaction mixture was stirred at room temperature for 2 hours and concentrated in vacuo. The residue was purified by column chromatography (increasing amount of MeOH in DCM) to give 2-imidazol-1-yl-4-morpholin-4-yl-pyrido[3,2-d]pyrimidine-6-carboxylic acid N′-acetyl-hydrazide (300 mg, 68%). The latter was suspended in phosphorous oxychloride (4 mL) and stirred at 100° C. for 14 hours. The reaction mixture was concentrated in vacuo and the residue quenched with cold water. The solution was neutralized with concentrated sodium hydroxide and extracted with ethyl acetate (2×20 mL), the combined organic phase was washed with brine (2×20 mL), dried over sodium sulfate and concentrated in vacuo. The residue was purified by column chromatography (increasing amount of MeOH in DCM) to afford the title compound as a yellow solid.

¹H NMR (400 MHz, CDCl₃) δ 8.66 (s, 1H), 8.56 (s, 1H), 7.96 (s, 1H), 7.18 (s, 1H), 5.24 (m, 2H), 4.32 (m, 2H), 3.96 (t, J=4.8 Hz, 4H), 2.70 (s, 3H). HPLC (Method A): RT 2.57 min (purity 95%). MS (ES+): 399.0.

### Intermediate 14

### 8-Chloro-2-imidazol-1-yl-4-morpholin-4-yl-6-hydroxymethyl-pyrido[3,2-d]pyrimidine

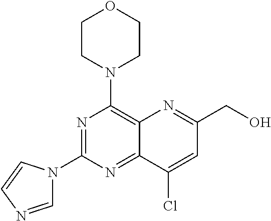

Lithium borohydride (115 mg; 5.3 mmol) was added at 0° C. to a suspension of Example 3 (1.0 g; 2.6 mmol) in ethanol (50 mL) and THF (50 mL) and the reaction mixture was stirred at room temperature for 2 hours. After concentration in vacuo, the residue was triturated in water, filtered off, washed with water and dried to afford the title compound (0.8 g, 86%) as a yellow solid.

¹H NMR (400 MHz, DMSO-d6) δ 8.61 (s, 1H), 7.99 (s, 1H), 7.95 (s, 1H), 7.10 (s, 1H), 5.70 (br s, 1H), 5.10 (m, 4H), 4.64 (d, J=4.6 Hz, 2H), 3.78 (t, J=4.5 Hz, 4H). HPLC (Method A): RT 3.39 min (purity 94%). MS (ES+): 346.9.

### Intermediate 15

### 8-Chloro-2-imidazol-1-yl-6-methoxymethyl-4-morpholin-4-yl-pyrido[3,2-d]pyrimidine

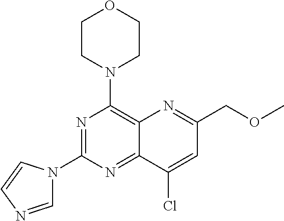

Sodium hydride (60% in mineral oil; 150 mg; 3.8 mmol) was added at 0° C. to a solution of Intermediate 14 (670 mg; 1.9 mmol) in DMF (10 mL) and the reaction mixture was stirred for 30 minutes whereupon methyl iodide (540 mg; 3.8 mmol) was added. The reaction mixture was stirred at room temperature for a further 2 hours then concentrated in vacuo. The residue was purified by column chromatography (increasing amount of MeOH in DCM) to afford the title compound as a yellow solid.

¹H NMR (400 MHz, CDCl₃) δ 8.63 (s, 1H), 7.95 (s, 1H), 7.90 (s, 1H), 7.16 (s, 1H), 4.93 (m, 4H), 4.6222H, s), 4.21-4.34 (2H, m), 16 (s, 2H), 3.93 (t, J=4.8 Hz, 4H), 3.52 (s, 3H). HPLC (Method C): RT 3.09 min (purity 87%). MS (ES+): 361.0.

### Intermediate 16

### Methyl 4-morpholin-4-yl-2-(1H-pyrazol-1-yl)pyrido[3,2-d]pyrimidine-6-carboxylate

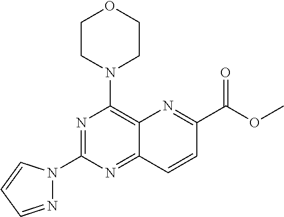

10% Pd on charcoal (400 mg) was added to a solution of Example 4 (1 g; 2.7 mmol) and ammonium formate (1.68 g; 27 mmol) in ethanol and the reaction mixture was stirred at reflux for 16 hours. The suspension was filtered through a short plug of Celite® which was further washed with a mixture of DCM and ethanol (1:1, 100 mL). Concentration in vacuo afforded the title compound along with the corresponding ethyl ester (800 mg) as a yellow solid which was used without further purification for the next step.

### Intermediate 17

### 4-Morpholin-4-yl-2-(1H-pyrazol-1-yl)pyrido[3,2-d]pyrimidine-6-carboxylic acid

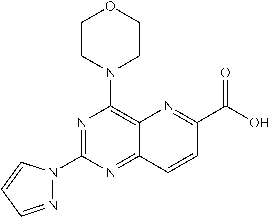

Lithium hydroxide (118 mg; 2.82 mmol) was added to a solution of Intermediate 16 (800 mg) in a mixture of methanol/tetrahydrofurane/water (3:2:1, 50 mL) and the reaction was stirred at 70° C. for 1 hour. After concentration in vacuo, the residue was quenched with cold water and the solution neutralized with aq. citric acid. The precipitate was filtered off, washed with cold water and dried to afford the title compound as a pale yellow solid.

HPLC (Method A): RT 4.03 min (purity 94%). MS (ES+): 327.0.

### Intermediate 18

### 8-Chloro-4-morpholin-4-yl-2-(1H-pyrazol-1-yl)pyrido[3,2-d]pyrimidine-6-carboxylic acid

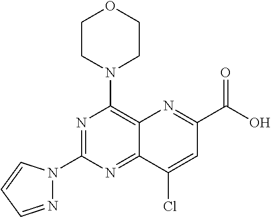

Lithium hydroxide (85 mg; 2.1 mmol) was added to a solution of Example 4 (650 mg; 1.7 mmol) in a mixture of methanol/tetrahydrofurane/water (3:2:1, 50 mL) and the reaction was stirred at 70° C. for 1 hour. After concentration in vacuo, the residue was quenched with cold water and the solution neutralized with aq. citric acid. The precipitate was filtered off, washed with cold water and dried to afford the title compound (520 mg, 76%) as an off white solid.

HPLC (Method D): RT 4.90 min (purity 84%). MS (ES+): 360.9.

### Intermediate 19

### 8-Chloro-6-(3-methyl-1,2,4-oxadiazol-5-yl)-4-morpholin-4-yl-2-(1H-pyrazol-1-yl)pyrido[3,2-d]pyrimidine

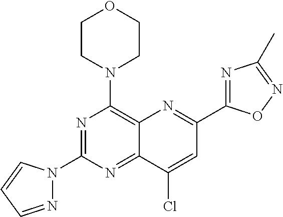

CDI (174 mg; 1.1 mmol) was added to a suspension of Intermediate 18 (190 mg; 0.53 mmol) in DMF (20 mL) and the resulting mixture was stirred at room temperature for 14 hours. After concentration in vacuo, the residue was taken up in DMF (3 mL) and toluene (40 mL) and N-hydroxy-acetamidine (58 mg; 0.79 mmol) was added. The reaction was stirred at reflux for 16 hours using a Dean Stark apparatus then concentrated in vacuo. The residue was purified by column chromatography (increasing amount of MeOH in DCM) to afford the title compound as a yellow solid.

¹H NMR (400 MHz, CDCl₃) δ 8.70 (m, 1H), 8.55 (s, 1H), 7.88 (m, 1H), 6.52 (m, 1H), 5.20 (m, 2H), 4.34 (m, 2H), 3.98 (t, J=4.8 Hz, 4H), 2.54 (s, 3H). HPLC (Method D): RT 5.57 min (purity 77%). MS (ES+): 399.0.

### Intermediate 20

### Methyl 2-(3-Hydroxymethyl-phenyl)-8-thiomethyl-4-morpholin-4-yl-pyrido[3,2-d]pyrimidine-6-carboxylate

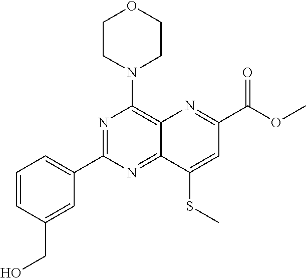

A suspension of Example 2 (200 mg; 0.56 mmol), 3-hydroxy methyl phenylboronic acid (250 mg; 1.6 mmol), Pd(PPh3)4 (64 mg; 0.06 mmol), sodium carbonate (170 mg; 1.6 mmol) in dioxane (12 mL) and water (12 mL) was stirred at 90° C. for 12 hours, filtered through a short plug of Celite® then concentrated in vacuo to afford the title compound as a yellow solid which was used without further purification.

MS (ES+): 427.0.

### Intermediate 21

### 2-[3-(Hydroxymethyl)phenyl]-8-(methylthio)-4-morpholin-4-ylpyrido[3,2-d]pyrimidine-6-carboxylic acid

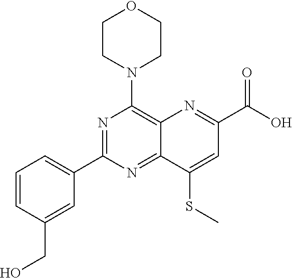

Lithium hydroxide (30 mg; 0.36 mmol) was added to a solution of Intermediate 20 (120 mg; 0.28 mmol) in a mixture of methanol/tetrahydrofurane/water (3:2:1, 20 mL), and the reaction mixture was stirred at 50° C. for 30 min. After concentration in vacuo, the residue was taken up in water and the solution acidified with aq. citric acid. The precipitate was filtered off, washed with water and dried to afford the title compound as a yellow solid

HPLC (Method D): RT 4.16 min (purity 76%). MS (ES+): 413.0.

### Intermediate 22

### Methyl 2-(1H-Indazol-4-yl)-8-thiomethyl-4-morpholin-4-yl-pyrido[3,2-d]pyrimidine-6-carboxylate

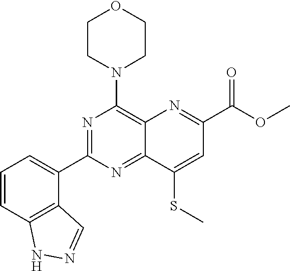

A suspension of Example 2 (200 mg; 0.56 mmol), indazole-4-boronic acid (180 mg; 1.12 mmol), Pd(PPh₃)₄ (64 mg; 0.06 mmol), sodium carbonate (170 mg; 1.6 mmol) in dioxane (12 mL) and water (12 mL) was stirred at 90° C. for 12 hours, filtered through a short plug of Celite® then concentrated in vacuo to afford the title compound as a yellow solid which was used without further purification.

HPLC (Method C): RT 2.47 min (purity 68%). MS (ES+): 437.0.

### Intermediate 23

### 2-(1H-Indazol-4-yl)-8-(methylthio)-4-morpholin-4-ylpyrido[3,2-d]pyrimidine-6-carboxylic acid

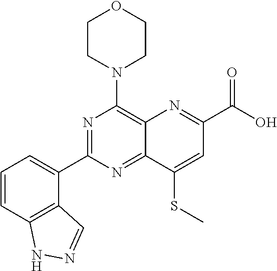

Lithium hydroxide (10 mg; 0.36 mmol) was added to a solution of Intermediate 22 (140 mg; 0.32 mmol) in a mixture of methanol/tetrahydrofurane/water (3:2:1, 20 mL) and the reaction mixture was stirred at 50° C. for 30 min. After concentration in vacuo, the residue was taken up in water and the solution acidified with aq. citric acid. The precipitate was filtered off, washed with water and dried to afford the title compound as a yellow solid.

HPLC (Method D): RT 2.50 min (purity 77%). MS (ES+): 423.0.

### Example 1

### Methyl 2,8-dichloro-4-morpholin-4-yl-pyrido[3,2-d]pyrimidine-6-carboxylate

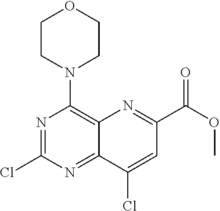

Morpholine (745 mg; 8.55 mmol; 1 eq.) was added, at 0° C., to a suspension of Intermediate 3 (2.5 g; 8.55 mmol; 1 eq.) and DIEA (3.31 g; 25.64 mmol; 3 eq.) in ACN (25 mL) and the reaction mixture was stirred at 0° C. for 2 hours. The solvent was evaporated in vacuo and the residue triturated in methanol. Filtration afforded the title compound (2.3 g, 79%) as a brown solid.

mp 212-213° C. (decomp.). ¹H NMR (300 MHz, DMSO-d₆) δ 8.46 (s, 1H), 4.98 (br s, 2H), 4.06 (br s, 2H), 3.93 (s, 3H), 3.85-3.76 (m, 4H). HPLC (Method A): RT 3.79 min (purity 98%). MS (ES+): 343.2. Anal. calcd. for C₁₃H₁₂Cl₂N₄O₃: C, 45.50; H, 3.52; N, 16.33. Found: C, 45.24; H, 3.66; N, 16.13.

### Example 2

### Methyl 2-chloro-8-methylsulfanyl-4-morpholin-4-yl-pyrido[3,2-d]pyrimidine-6-carboxylate

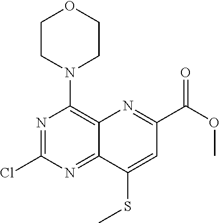

A suspension of sodium thiomethoxide (552 mg; 7.87 mmol; 0.9 eq.) in THF (20 mL) was added to a solution of Example 1 (3 g; 8.74 mmol; 1 eq.) in THF (30 mL). The reaction mixture was stirred at room temperature for 10 hours whereupon sodium thiomethoxide (61 mg; 0.87 mmol; 0.1 eq) was added and the mixture was stirred at room temperature for three hours. Sodium thiomethoxide (61.3 mg; 0.87 mmol; 0.1 eq) was added and the reaction mixture was stirred for another 16 hours. Water (40 mL) was added and the resulting precipitate was filtered off and dried. The residue was triturated in methanol, filtered and dried under vacuum to afford the title compound (2.53 g, 84%) as a yellow solid.

¹H NMR (300 MHz, CDCl₃) δ 7.98 (s, 1H), 5.09 (br s, 2H), 4.18 (br s, 2H), 4.00 (s, 3H), 4.15-3.68 (m, 4H), 2.55 (s, 3H). HPLC (Method A): RT 3.94 min (purity 92%). MS (ES+): 355.2.

### Example 3

### Methyl 8-chloro-2-(1H-imidazol-1-yl)-4-morpholin-4-ylpyrido[3,2-d]pyrimidine-6-carboxylate

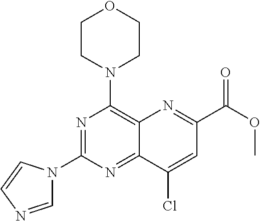

A suspension of Example 1 (343 mg; 1 mmol; 1 eq.), imidazole (75 mg; 1.1 mmol; 1.1 eq.) and K₂CO₃ (276 mg; 2 mmol; 2 eq.) in DMA (2 mL) was stirred at room temperature for 2 hours then at 50° C. for 16 hours. Imidazole (21 mg; 0.31 mmol; 0.31 eq.) and K₂CO₃ (86 mg; 0.62 mmol; 0.62 eq.) were added and the reaction mixture was stirred at 60° C. for a further 16 hours. After dilution with sat. aq. NH₄Cl, the precipitate was filtered off, washed thoroughly with water then MeOH and Et₂O to give a pinkish solid. Recrystallization from DCM/n-pentane afforded the title compound as an off-white solid.

¹H NMR (300 MHz, DMSO-d₆) δ 8.68 (s, 1H), 8.45 (s, 1H), 8.00 (t, J=2.3 Hz, 1H), 7.14 (br t, J=1.0 Hz, 1H), 5.03 (br s, 2H), 4.23 (br s, 2H), 3.94 (s, 3H), 3.82 (br t, J=4.6 Hz, 4H). HPLC (Method A): RT 2.38 min (purity 95%). MS (ES+): 375.2.

### Example 4

### Methyl 8-chloro-4-morpholin-4-yl-2-(1H-pyrazol-1-yl)pyrido[3,2-d]pyrimidine-6-carboxylate

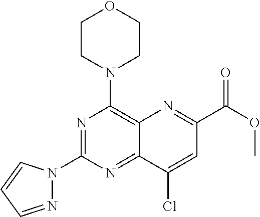

A mixture of Example 1 (164 mg; 0.48 mmol; 1 eq.), pyrazole (65 mg; 0.96 mmol; 2 eq.) and K₂CO₃ (330 mg; 2.39 mmol; 5 eq.) in DMF (3 mL) was stirred at 60° C. for 20 hours. The solvent was evaporated in vacuo and the residue partitioned between sat. aq. NH₄Cl and DCM. The organic phase was dried over MgSO₄, concentrated in vacuo and the residue triturated in DCM. The insoluble material was removed by filtration and the solution evaporated to dryness. The residue was purified by column chromatography (increasing amount of ethyl acetate in c-hexane) to afford the title compound as a yellow foam.

¹H NMR (300 MHz, CDCl₃) δ 8.73 (d, J=2.4 Hz, 1H), 8.46 (s, 1H), 7.93 (d, J=1.2 Hz, 1H), 6.55 (dd, J=2.4, 1.2 Hz, 1H), 5.15 (br s, 2H), 4.39 (br s, 2H), 4.04 (s, 3H), 3.97 (t, J=4.7 Hz, 4H). HPLC (Method A): RT 3.97 min (purity 99%). MS (ES+): 375.2.

### Example 5

### Methyl 4-morpholin-4-yl-2,8-di-1H-pyrrol-1-ylpyrido[3,2-d]pyrimidine-6-carboxylate

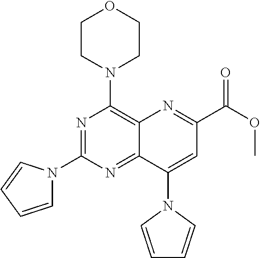

NaH (21 mg; 0.87 mmol; 2 eq.) was added to a solution of pyrrole (61 μL; 0.87 mmol; 2 eq.) in DMA and the resulting mixture was stirred at room temperature for 10 min whereupon a solution of Example 1 (150 mg; 0.44 mmol; 1 eq.) in DMA (10 mL) was added. The reaction mixture was stirred at 120° C. for 30 min (microwave heating) and the solvent was removed in vacuo. Purification mass triggered preparative HPLC (increasing amount of ACN in water as eluent) afforded the corresponding carboxylic acid (34 mg) as a brown solid. To the acid (34 mg; 0.08 mmol; 1 eq.) in MeOH (3 mL), was added thionyl chloride (18 μL; 0.25 mmol; 3 eq.) and the resulting mixture was stirred at room temperature for 3 hours. After evaporation of the solvent, the residue was taken up in DCM, washed with water, dried over magnesium sulfate and concentrated in vacuo to afford the title compound as a yellow solid.

¹H NMR (300 MHz, CDCl₃) δ 8.23 (br s, 1H), 7.78-7.70 (m, 4H), 6.46-6.39 (m, 2H), 6.37-6.29 (m, 2H), 5.20-4.15 (m, 4H), 3.95 (s, 3H), 3.91-3.75 (m, 4H). HPLC (Method A): RT 5.20 min (purity 92%). MS (ES+): 405.2.

### Example 6

### Methyl 8-(methylthio)-4-morpholin-4-yl-2-(3-thienyl)pyrido[3,2-d]pyrimidine-6-carboxylate

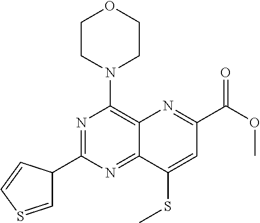

A suspension of Example 2 (150 mg; 0.42 mmol; 1 eq.), 3-thienylboronic acid (60 mg; 0.47 mmol; 1.1 eq.), Pd(PPh₃)₄ (24 mg; 0.02 mmol; 0.05 eq.) and Cs₂CO₃ (413 mg; 1.27 mmol; 3 eq.) in Dioxane (3 mL) was stirred at 90° C. for 7 hours. The reaction mixture was allowed to return to room temperature and was then partitioned between DCM and water. The two phases were separated and the aqueous layer was extracted with DCM (2×). The combined organic phase was dried over sodium sulphate and filtered through a short plug of Celite®. After evaporation of the solvent, purification by column chromatography (DCM/MeOH, 98/2) followed by recrystallization from DCM/n-pentane afforded the title compound as a pale yellow solid.

¹H NMR (300 MHz, DMSO-d₆) δ 8.41 (dd, J=3.1, 1.1 Hz, 1H), 7.93 (s, 1H), 7.82 (dd, J=5.0, 1.1 Hz, 1H), 7.64 (dd, J=5.0, 3.1 Hz, 1H), 4.60 (br s, 4H), 3.92 (s, 3H), 3.81 (br t, J=4.6 Hz, 4H), 2.56 (s, 3H). HPLC (Method A): RT 4.70 min (purity 99%). MS (ES+): 403.2.

### Example 7

### Methyl 8-(methylthio)-4-morpholin-4-yl-2-phenylpyrido[3,2-d]pyrimidine-6-carboxylate

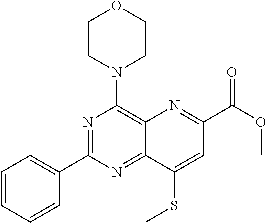

A suspension of phenylboronic acid (344 mg; 2.82 mmol; 1 eq.), Example 2 (1 g; 2.82 mmol; 1 eq.), Cs₂CO₃ (2.75 g; 8.46 mmol; 3 eq.) and Pd(PPh₃)₄ (163 mg; 0.14 mmol; 0.05 eq.) in dioxane (22.5 mL) was stirred at 90° C. for 16 h. DCM was added to the reaction mixture and the organic phase was washed with water then brine, dried over sodium sulphate and filtered through a short plug of Celite®. Solvents were removed under vacuum and the resulting residue was triturated in MeOH, filtered and washed with MeOH then Et₂O to afford the title compound (990 mg, 89%) as a yellow solid.

¹H NMR (300 MHz, CDCl₃) δ8.56-8.53 (m, 2H), 8.02 (s, 1H), 7.49-7.47 (m, 3H), 4.69 (br s, 4H), 4.01 (s, 3H), 3.99-3.92 (m, 4H), 2.59 (s, 3H). HPLC (Method A): RT 5.06 min (purity 96%). MS (ES+): 397.3.

### Example 8

### Methyl 2-(1H-indol-4-yl)-8-(methylthio)-4-morpholin-4-ylpyrido[3,2-d]pyrimidine-6-carboxylate

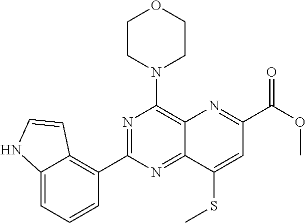

To a suspension of Example 2 (80 mg; 0.23 mmol; 1 eq.), 1-(tert-butyldimethylsilyl)-1H-indol-4-ylboronic acid (68 mg; 0.25 mmol; 1.1 eq.) and sodium carbonate (72 mg; 0.68 mmol; 3) in toluene (2.5 mL), EtOH (1.5 mL) and water (0.7 ml) was added Pd(PPh₃)₂Cl₂ (8 mg; 0.01 mmol; 0.05 eq.) and the reaction mixture was stirred at 120° C. for 1 h (microwave heating). The solution was diluted with 5% MeOH in DCM and filtered through a short of Celite®, dried over magnesium sulphate and evaporated in vacuo. The residue was taken up in MeOH (5 mL), one drop of conc. H₂SO₄ was added and the mixture was heated at reflux for 16 hours. The solution was concentrated in vacuo, taken up in DCM and washed with water. The organic layer was dried over magnesium sulfate, filtered and evaporated in vacuo. The residue was purified by column chromatography using increasing amount of ethyl acetate in c-hexane to afford the title compound as a yellow solid.

¹H NMR (300 MHz, DMSO-d₆) δ 11.33 (br s, 1H), 8.30 (dd, J=7.6, 0.9 Hz, 1H), 8.02 (s, 1H), 7.78 (t, J=2.1 Hz, 1H), 7.60 (d, J=8.0 Hz, 1H), 7.53 (t, J=2.7 Hz, 1H), 7.24 (t, J=7.8 Hz, 1H), 3.90 (s, 3H), 3.88-3.83 (m, 4H), 3.32 (s, 2H), 2.65 (s, 3H), 1.39 (s, 2H). HPLC (Method A): RT 5.23 min (purity 96%). MS (ES+): 436.1 (ES−): 434.2

### Example 9

### Methyl 2-(1-methyl-1H-pyrazol-4-yl)-8-(methylthio)-4-morpholin-4-ylpyrido[3,2-d]pyrimidine-6-carboxylate

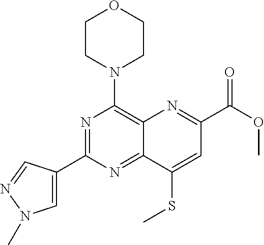

To a suspension of Example 2 (110 mg; 0.31 mmol; 1 eq.), 1-methyl-4-(4,4,5,5-tetramethyl-1,3,2-dioxaborolan-2-yl)-1H pyrazole (71 mg; 0.34 mmol; 1.1 eq.) and cesium carbonate (303 mg; 0.93 mmol; 3 eq.) in dioxane (5.00 ml) was added Pd(PPh₃)₄ (18 mg; 0.02 mmol; 0.05 eq.) and the reaction mixture was stirred at 150° C. for 1 h (microwave heating). The suspension was filtered and the solvent was evaporated in vacuo. The residue was purified by column chromatography using increasing amount of ethyl acetate in c-hexane followed by trituration in MeOH to afford the title compound as a beige solid.

¹H NMR (300 MHz, DMSO-d₆) δ 8.35 (s, 1H), 8.01 (s, 1H), 7.91 (s, 1H), 4.90-4.36 (br s, 4H), 3.90 (s, 6H), 3.78 (br s, 4H), 2.54 (s, 3H). HPLC (Method A): RT 3.25 min (purity 95%). MS (ES+): 401.1.

### Example 10

### Methyl 8-(methylthio)-4-morpholin-4-yl-2-(1H-pyrazol-4-yl)pyrido[3,2-d]pyrimidine-6-carboxylate

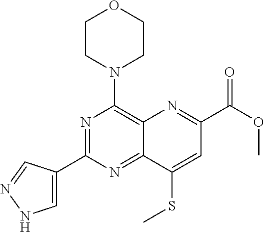

To a suspension of Example 2 (110 mg; 0.31 mmol; 1 eq.), pyrazole-4-boronic acid (38 mg; 0.34 mmol; 1.1 eq.) and cesium carbonate (303 mg; 0.93 mmol; 3 eq.) in dioxane (5.00 ml) was added Pd(PPh₃)₄ (18 mg; 0.02 mmol; 0.05 eq.) and the reaction mixture was stirred at 150° C. for 1 h (microwave heating). The suspension was filtered and the solvent was evaporated in vacuo. The residue was purified by column chromatography using increasing amount of ethyl acetate in c-hexane then 50% MeOH in DCM to afford the title compound as a yellow solid.

¹H NMR (300 MHz, DMSO-d₆) δ 8.66 (s, 1H), 7.95 (s, 1H), 7.83 (s, 1H), 6.57 (s, 1H), 3.91 (s, 3H), 3.88 (br s, 4H), 2.57 (s, 3H), 3.31 (s, 4H). HPLC (Method A): RT 3.48 min (purity 99%). MS (ES+): 387.1.

### Example 11

### Methyl 8-(methylthio)-4-morpholin-4-yl-2-pyridin-4-ylpyrido[3,2-d]pyrimidine-6-carboxylate. HCl

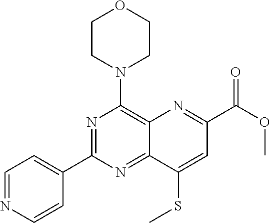

To a suspension of Example 2 (60 mg; 0.17 mmol; 1 eq.), 4-pyridineboronic acid (23 mg; 0.19 mmol; 1.1 eq.), and cesium carbonate (165 mg; 0.51 mmol; 3 eq.) in dioxane (5 mL) was added Pd(PPh₃)₄ (10 mg; 0.01 mmol; 0.05 eq.) and the reaction mixture was stirred at 150° C. for 1 hour (microwave heating). The suspension was filtered through a short plug of Celite® and the solvent was evaporated in vacuo. The residue was purified by preparative HPLC (increasing amount of 0.1% TFA in CH₃CN, in 0.1% TFA in water). Fractions containing the compound were combined, 1M HCl (0.1 mL) was added and the solution was freeze dried to afford the title compound as an orange solid.

¹H NMR (300 MHz, DMSO-d₆) δ 8.97 (d, J=6.5 Hz, 2H), 8.65 (d, J=6.5 Hz, 2H), 8.01 (s, 1H), 5.40-4.10 (m, 4H), 3.98 (s, 3H), 3.84 (br t, J=4.5 Hz, 4H), 2.62 (s, 3H). HPLC (Method A): RT 2.64 min (purity 96%). MS (ES+): 398.1.

### Example 12

### Methyl 2-(1H-imidazol-1-yl)-8-(methylthio)-4-morpholin-4-ylpyrido[3,2-d]pyrimidine-6-carboxylate

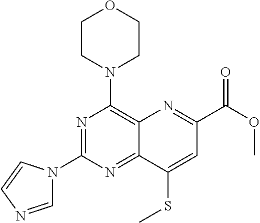

A mixture of Example 2 (378 mg; 1.07 mmol; 1 eq.), imidazole (145 mg; 2.13 mmol; 2 eq.) and potassium carbonate (736 mg; 5.33 mmol; 5 eq.) in DMA (5 mL) was stirred at room temperature for 2 hours then at 60° C. for 2 hours. The reaction mixture was allowed to return to room temperature and poured into sat. aq. NH₄Cl. The precipitate was filtered, washed with water and dried under vacuum. Recrystallization from DCM/n-pentane afforded the title compound as an off-white solid.

¹H NMR (300 MHz, DMSO-d₆) δ 8.60 (t, J=1.1 Hz, 1H), 7.93 (s, 1H), 7.92 (t, J=1.3 Hz, 1H), 7.12 (m, 1H), 5.05 (br s, 2H), 4.19 (br s, 2H), 3.93 (s, 3H), 3.81 (br t, J=4.7 Hz, 4H), 2.57 (s, 3H). HPLC (Method A): RT1.99 min (purity 97%). MS (ES+): 387.2.

### Example 13

### Methyl 2-chloro-8-(methylsulfonyl)-4-morpholin-4-ylpyrido[3,2-d]pyrimidine-6-carboxylate

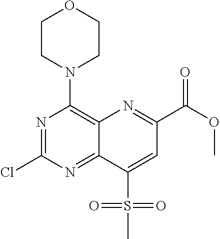

m-CPBA (97.3 mg; 0.56 mmol; 2 eq.) was added to a mixture of Example 2 (100 mg; 0.28 mmol; 1 eq.) and NaHCO₃ (118.4 mg; 1.41 mmol; 5 eq.) in DCM (10 mL) and water (5 mL) and the resulting mixture was stirred at room temperature for 7 hours, whereupon m-CPBA (30 mg; 0.17 mmol; 0.62 eq.) was added. The reaction mixture was stirred for a further 16 hours. m-CPBA (30 mg; 0.17 mmol; 0.62 eq.) was added and the reaction mixture was stirred for a further 4 hours. The two phases were separated and the aqueous layer was extracted with DCM (2×). The combined organic phase was washed with sat. aq. NaHCO₃ then brine, dried over sodium sulphate and concentrated in vacuo. Recrystallization from DCM/n-pentane afforded the title compound as a bright yellow solid.

¹H NMR (300 MHz, DMSO-d₆) δ 8.62 (s, 1H), 4.96 (br s, 2H), 4.11 (br s, 2H), 3.97 (s, 3H), 3.81 (t, J=5.1 Hz, 4H), 3.58 (s, 3H). HPLC (Method A): RT 3.29 min (purity 98%). MS (ES+): 387.2.

### Example 14

### Methyl 2-methyl-8-(methylthio)-4-morpholin-4-ylpyrido[3,2-d]pyrimidine-6-carboxylate

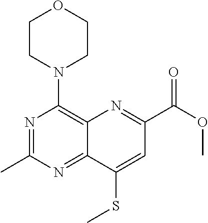

A suspension of Example 2 (500 mg; 1.41 mmol; 1 eq.), methylboronic acid (126 mg; 2.11 mmol; 1.5 eq.), Pd(PPh₃)₄ (81 mg; 0.07 mmol; 0.05 eq.) and Cs₂CO₃ (1.38 g; 4.23 mmol; 3 eq.) in dioxane (10 mL) was stirred at 90° C. for 14 hours. The reaction mixture was allowed to return to room temperature and was then partitioned between DCM and water. The two phases were separated and the aqueous layer was extracted with DCM (2×). The combined organic phase was dried over sodium sulphate and filtered through a short plug of Celite®. After evaporation of the solvent, purification by column chromatography (DCM then DCM/MeOH, 98/2) followed by recrystallization from ethyl acetate afforded the title compound as a yellow solid.

¹H NMR (300 MHz, DMSO-d₆) δ 7.93 (s, 1H), 4.49 (br s, 4H), 3.92 (s, 3H), 3.77 (br t, J=4.5 Hz, 4H), 2.53 (s, 3H), 2.50 (s, 3H). HPLC (Method A): RT1.98 min (purity 88%). MS (ES+): 335.3.

### Example 15

### Methyl 2-(1H-imidazol-1-yl)-4,8-dimorpholin-4-ylpyrido[3,2-d]pyrimidine-6-carboxylate

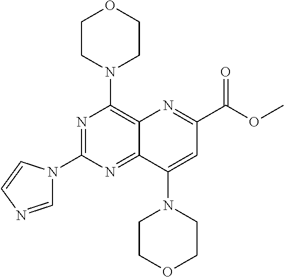

A mixture of Example 3 (150 mg; 0.4 mmol; 1 eq.) and morpholine (0.8 mL) was stirred at 75° C. for 4 hours then diluted with 5% aq. AcOH. The precipitate was filtered, washed thoroughly with water and dried. The residue was recrystallized from DCM/MeOH then washed successively with MeOH, Et₂O and n-pentane to afford the title compound as a beige solid.

¹H NMR (300 MHz, DMSO-d₆) δ 8.57 (br s, 1H), 7.89 (t, J=1.2 Hz, 1H), 7.56 (s, 1H), 7.12 (br s, 1H), 4.49 (br s, 2H), 3.90 (br s, 2H), 3.89-3.84 (m, 4H), 3.84-3.76 (m, 4H), 3.67-3.59 (m, 4H). HPLC (Method A): RT 2.28 min (purity 96%). MS (ES+): 426.2.

### Example 16

### Methyl 2-(1H-imidazol-1-yl)-4-morpholin-4-ylpyrido[3,2-d]pyrimidine-6-carboxylate

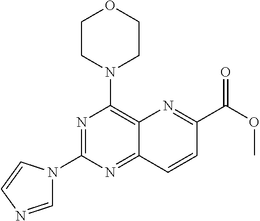

A mixture of Example 3 (100 mg; 0.27 mmol; 1 eq.), ammonium formate (336 mg; 5.34 mmol; 20 eq.) and 10% Pd/C (30 mg) in EtOH (20 mL) was stirred at reflux for 45 minutes then concentrated in vacuo. The residue was taken up in 5% MeOH in DCM and filtered through a short plug of Celite® which was further washed with 5% MeOH in DCM. The solution was concentrated in vacuo and the residue was recrystallized from MeOH to afford the title compound as an off-white solid.

¹H NMR (300 MHz, DMSO-d₆) δ 8.66 (br s, 1H), 8.32 (d, J=8.7 Hz, 1H), 8.20 (d, J=8.7 Hz, 1H), 7.99 (t, J=1.3 Hz, 1H), 7.13-7.10 (m, 1H), 5.07 (br s, 2H), 4.21 (br s, 2H), 3.93 (s, 3H), 3.83 (br t, J=4.7 Hz, 4H). HPLC (Method A): RT1.70 min (purity 97%). MS (ES+): 341.2.

### Example 17

### [8-(Methylthio)-4-morpholin-4-yl-2-(3-thienyl)pyrido[3,2-d]pyrimidin-6-yl]methanol

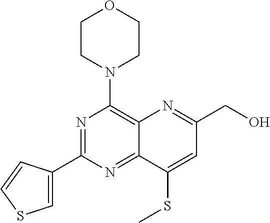

A mixture of Example 6 (73 mg; 0.18 mmol; 1 eq.) and lithium borohydride (4 mg; 0.18 mmol; 1 eq.) in THF (1 mL) and EtOH (1 mL) was stirred at room temperature for 2 hours whereupon lithium borohydride (16 mg; 0.72 mmol; 4 eq.) was added. The reaction mixture was stirred at room temperature for a further 3 hours and diluted with water. The aqueous layer was extracted with DCM (3×), the combined organic phase dried over magnesium sulphate and concentrated in vacuo. Crystallization from DCM/n-pentane afforded the title compound as an off-white solid.

¹H NMR (300 MHz, DMSO-d₆) δ8.33 (dd, J=3.0, 1.1 Hz, 1H), 7.82 (dd, J=5.0, 1.2, Hz 1H), 7.62 (dd, J=5.0, 3.1 Hz, 1H), 7.59 (s, 1H), 5.59 (t, J=6.0 Hz, 1H), 4.63 (d, J=5.9 Hz, 2H), 4.49 (br s, 4H), 3.79 (br t, J=4.8 Hz, 4H), 2.51 (s, 3H). HPLC (Method A): RT 3.50 min (purity 98%). MS (ES+): 375.2.

### Example 18

### Methyl 4-morpholin-4-yl-2-phenylpyrido[3,2-d]pyrimidine-6-carboxylate. Trifluoroacetate salt

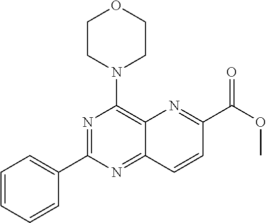

A solution of Example 7 (50 mg; 0.10 mmol; 1 eq.) in MeOH (15 mL) was passed through a Raney Nickel cartridge in a H-Cube apparatus (1 mL/min; room temperature; full H₂ mode). The solvent was removed in vacuo and the residue was purified by preparative HPLC (increasing amount of 0.1% TFA in CH₃CN, in 0.1% TFA in water) to afford the title compound as a white fluffy solid.

¹H NMR (300 MHz, DMSO-d₆) δ 8.50-8.43 (m, 2H), 8.37 (d, J=8.7 Hz, 1H), 8.32 (d, J=8.7 Hz, 1H), 7.60-7.51 (m, 3H), 4.66 (br s, 4H), 3.94 (s, 3H), 3.85 (br t, J=4.8 Hz, 4H). HPLC (Method A): RT 2.54 min (purity 100%). MS (ES+): 351.2.

### Example 19

### Methyl 2-(1H-imidazol-1-yl)-8-(methylsulfonyl)-4-morpholin-4-ylpyrido[3,2-d]pyrimidine-6-carboxylate

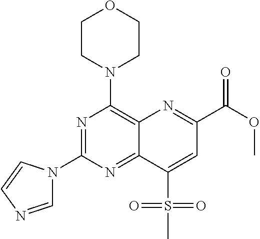

A suspension of Example 13 (160 mg; 0.41 mmol; 1 eq.), imidazole (34 mg; 0.5 mmol; 1.2 eq.) and K₂CO₃ (114 mg; 0.83 mmol; 2 eq.) in DMA (1 mL) was stirred at room temperature for 16 hours. The reaction mixture was then diluted with sat. aq. NH₄Cl and the precipitate filtered off. The solid was washed successively with water, MeOH and Et₂O and dried under vacuum. Purification by column chromatography (DCM/MeOH, 98/2) afforded the title compound as a pale yellow solid.

¹H NMR (300 MHz, DMSO-d₆) δ 8.70 (s, 1H), 8.63 (s, 1H), 8.00 (s, 1H), 7.17 (s, 1H), 5.01 (br s, 2H), 4.28 (br s, 2H), 3.98 (s, 3H), 3.88-3.77 (m, 4H), 3.63 (s, 3H). HPLC (Method A): RT 1.72 min (purity 100%). MS (ES+): 419.2.

### Example 20

### [2-(1H-Imidazol-1-yl)-4-morpholin-4-ylpyrido[3,2-d]pyrimidin-6-yl]methanol

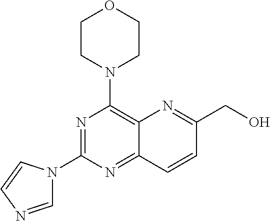

Lithium borohydride (29 mg; 1.32 mmol; 5 eq.) was carefully added to a suspension of Example 16 (90 mg; 0.26 mmol; 1 eq.) in MeOH (15 mL) and the resulting mixture was stirred at 60° C. for 24 hours. The solution was concentrated in vacuo and the residue partitioned between DCM and water. The aqueous layer was extracted twice with DCM and the combined organic phase washed with brine, dried over magnesium sulphate and concentrated in vacuo. The residue was purified by column chromatography (DCM/MeOH, 96/4) followed by crystallization from Et₂O to afford the title compound as a white solid.

¹H NMR (300 MHz, DMSO-d₆) δ 8.62 (t, J=1.0 Hz, 1H), 8.12 (d, J=8.7 Hz, 1H), 7.97 (t, J=1.3 Hz, 1H), 7.88 (d, J=8.7 Hz, 1H), 7.11-7.08 (m, 1H), 5.63 (t, J=6.0 Hz, 1H), 4.67 (d, J=6.0 Hz, 2H), 4.56 (br s, 4H), 3.80 (br t, J=4.7 Hz, 4H). HPLC (Method A): RT1.43 min (purity 97%). MS (ES+): 313.1, (ES−): 311.2.

### Example 21

### 2-(1H-Imidazol-1-yl)-N,N-dimethyl-4-morpholin-4-ylpyrido[3,2-d]pyrimidine-6-carboxamide

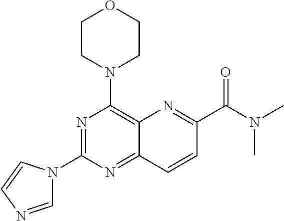

To a solution of Example 16 (800 mg; 2.35 mmol; 1 eq.) in THF (3 mL) was added dropwise 1M NaOH (3 mL; 3 mmol; 1.28 eq.) and the resulting mixture was stirred at room temperature for 2 hours. The THF was evaporated in vacuo and the residue diluted with water. The pH was made acidic with 1M HCl and the solution washed successively with DCM and ethyl acetate. The aqueous layer was cooled down to 4° C. until precipitation. The solid was collected by filtration, washed with water then Et₂O to give the corresponding acid (480 mg, 63%) as a white solid. The acid (150 mg; 0.46 mmol; 1 eq.) was taken up in DCM (6 mL) and EDC (132 mg; 0.69 mmol; 1.5 eq.), 1-hydroxybenzotriazole (93 mg; 0.69 mmol; 1.5 eq.) and dimethylamine (2M in THF; 460 μL; 0.92 mmol; 2 eq.) were added. The reaction mixture was stirred at room temperature for 48 hours then diluted with DCM and washed successively with water and sat. aq. NH₄Cl. The organic phase was dried over magnesium sulphate and concentrated in vacuo to afford the title compound (124 mg, 76%) as a white solid.

¹H NMR (300 MHz, DMSO-d₆) δ 8.66 (t, J=1.0 Hz, 1H), 8.16 (d, J=8.7 Hz, 1H), 7.99 (t, J=1.3 Hz, 1H), 7.95 (d, J=8.7 Hz, 1H), 7.11 (br t, J=1.2 Hz, 1H), 5.13-4.04 (m, 4H), 3.81 (br t, J=4.8 Hz, 4H), 3.05 (s, 3H), 3.04 (s, 3H). HPLC (Method A): RT1.65 min (purity 98%). MS (ES+): 354.2.

### Example 22

### 2-(1H-Imidazol-1-yl)-N-methyl-4-morpholin-4-ylpyrido[3,2-d]pyrimidine-6-carboxamide

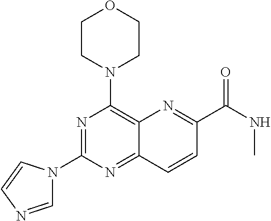

To a solution of Example 16 (800 mg; 2.35 mmol; 1 eq.) in THF (3 mL) was added dropwise 1M NaOH (3 mL; 3 mmol; 1.28 eq.) and the resulting mixture was stirred at room temperature for 2 hours. The THF was evaporated in vacuo and the residue diluted with water. The pH was made acidic with 1M HCl and the solution washed successively with DCM and ethyl acetate. The aqueous layer was cooled down to 4° C. until precipitation. The solid was collected by filtration, washed with water then Et₂O to give the corresponding acid (480 mg, 63%) as a white solid. The acid (150 mg; 0.46 mmol; 1 eq.) was taken up in DCM (6 mL) and EDC (132 mg; 0.69 mmol; 1.5 eq.), 1-hydroxybenzotriazole (93 mg; 0.69 mmol; 1.5 eq.) and methylamine (2M in THF; 460 μL; 0.92 mmol; 2 eq.) were added. The reaction mixture was stirred at room temperature for 48 hours then diluted with DCM and washed successively with water and sat. aq. NH₄Cl. The organic phase was dried over magnesium sulfate and concentrated in vacuo to afford the title compound (109 mg, 70%) as a white solid.

¹H NMR (300 MHz, DMSO-d₆) δ 8.66 (br s, 1H), 8.31 (d, J=8.6 Hz, 1H), 8.30-8.29 (m, 1H), 8.20 (d, J=8.6 Hz, 1H), 7.99 (br s, 1H), 7.11 (br s, 1H), 5.10-4.01 (m, 4H), 3.82 (br s, 4H), 2.89 (br d, J=4.0 Hz, 3H). HPLC (Method A): RT1.60 min (purity 99%). MS (ES+): 340.1 (ES−): 338.2.

### Example 23

### 2-(1H-Imidazol-1-yl)-4-morpholin-4-ylpyrido[3,2-d]pyrimidine-6-carboxamide

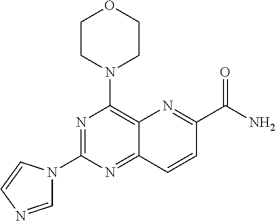

To a solution of Example 16 (800 mg; 2.35 mmol; 1 eq.) in THF (3 mL) was added dropwise 1M NaOH (3 mL; 3 mmol; 1.28 eq.) and the resulting mixture was stirred at room temperature for 2 hours. The THF was evaporated in vacuo and the residue diluted with water. The pH was made acidic with 1M HCl and the solution washed successively with DCM and ethyl acetate. The aqueous layer was cooled down to 4° C. until precipitation. The solid was collected by filtration, washed with water then Et₂O to give the corresponding acid (480 mg, 63%) as a white solid. The acid (170 mg; 0.52 mmol; 1 eq.) was taken up in DCM (6 mL) and EDC (150 mg; 0.78 mmol; 1.5 eq.), 1-hydroxybenzotriazole (106 mg; 0.78 mmol; 1.5 eq.) and ammonia (0.5M in dioxane; 5.2 mL; 2.6 mmol; 5 eq.) were added. The reaction was stirred at room temperature for 16 hours then diluted with DCM and washed successively with water and sat. aq. NH₄Cl. The organic phase was dried over magnesium sulfate and concentrated in vacuo. The residue was taken up in 10% MeOH in DCM and filtered through a short plug of alumina to afford the title compound as a white solid.

¹H NMR (300 MHz, DMSO-d₆) δ 8.66 (s, 1H), 8.33 (d, J=8.7 Hz, 1H), 8.20 (d, J=8.7 Hz, 1H), 8.00 (s, 1H), 7.86 (br s, 1H), 7.76 (br s, 1H), 7.11 (s, 1H), 4.85-4.07 (m, 4H), 3.82 (br s, 4H). HPLC (Method A): RT1.38 min (purity 98%). MS (ES+): 326.1 (ES−) 324.1.

### Example 24

### 1-[2-(1H-Imidazol-1-yl)-4-morpholin-4-ylpyrido[3,2-d]pyrimidin-6-yl]-N,N-dimethylmethanamine

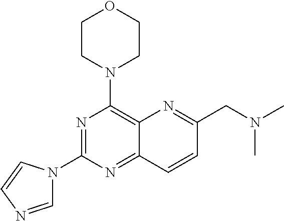

Methanesulfonyl chloride (44 mg; 0.38 mmol; 1.2 eq.) was added to a solution of Example 20 (100 mg; 0.32 mmol; 1 eq.) and DIEA (276 μl; 1.6 mmol; 5 eq.) in DCM (6 mL) and the resulting mixture was stirred at room temperature for 2 hours. Dimethylamine (2M in THF; 480 μL; 0.96 mmol; 3 eq.) was added and the reaction mixture was stirred at room temperature for 16 hours. The solution was diluted with DCM and washed with 5% aq. NaHCO₃. The organic phase was dried over magnesium sulphate and concentrated in vacuo. The residue was purified by mass triggered preparative HPLC (increasing amount of ACN in water as eluent) to afford the title compound as a white solid.

¹H NMR (300 MHz, DMSO-d₆) δ 8.62 (t, J=1.1 Hz, 1H), 8.08 (d, J=8.6 Hz, 1H), 7.97 (t, J=1.4 Hz, 1H), 7.84 (t, J=8.6 Hz, 1H), 7.10 (t, J=1.3 Hz, 1H), 5.26-4.09 (m, 4H), 3.80 (t, J=4.9 Hz, 4H), 3.65 (s, 2H), 2.22 (s, 6H). HPLC (Method A): RT1.19 min (purity 89%). MS (ES+): 340.2.

### Example 25

### 2-(1H-Imidazol-1-yl)-6-(methoxymethyl)-4-morpholin-4-ylpyrido[3,2-d]pyrimidine

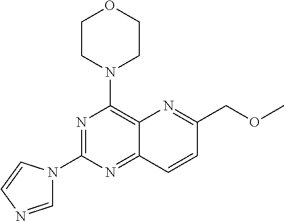

Methanesulfonyl chloride (44 mg; 0.38 mmol; 1.2 eq.) was added to a solution of Example 20 (100 mg; 0.32 mmol; 1 eq.) and DIEA (276 μl; 1.6 mmol; 5 eq.) in DCM (6 mL) and the resulting mixture was stirred at room temperature for 2 hours. The solvent was evaporated in vacuo and the residue taken up in MeOH (3 mL). The reaction mixture was stirred for 2 hours at 130° C. (microwave heating). The solvent was evaporated in vacuo and the residue diluted with DCM. The organic phase was washed with 5% aq. NaHCO₃ then 0.1M HCl, dried over magnesium sulphate and concentrated in vacuo. The residue was purified by mass triggered preparative HPLC (increasing amount of ACN in water as eluent) to afford the title compound as a white solid.

¹H NMR (300 MHz, DMSO-d₆) δ 8.63 (t, J=1.0 Hz, 1H), 8.12 (d, J=8.7 Hz, 1H), 7.98 (t, J=1.3 Hz, 1H), 7.82 (d, J=8.7 Hz, 1H), 7.10 (t, J=1.2 Hz, 1H), 5.14-4.09 (m, 4H), 4.63 (s, 2H), 3.80 (br t, J=4.7 Hz, 4H), 3.40 (s, 3H). HPLC (Method A): RT 2.18 min (purity 100%). MS (ES+): 327.2.

### Example 26

### 2-(1H-Imidazol-1-yl)-6-[(methylthio)methyl]-4-morpholin-4-ylpyrido[3,2-d]pyrimidine

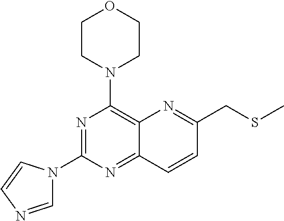

Methanesulfonyl chloride (44 mg; 0.38 mmol; 1.2 eq.) was added to a solution of Example 20 (100 mg; 0.32 mmol; 1 eq.) and DIEA (276 μl; 1.6 mmol; 5 eq.) in DCM (6 mL) and the resulting mixture was stirred at room temperature for 2 hours. Sodium thiomethoxide (67 mg; 0.96 mmol; 3 eq.) was added and the reaction mixture was stirred at room temperature for 16 hours. Sodium thiomethoxide (67 mg; 0.96 mmol; 3 eq.) was added and the reaction mixture was stirred at room temperature for a further 8 hours. The solution was diluted with DCM, washed with 5% aq. NaHCO₃ then 0.1 M HCl, dried over magnesium sulphate and concentrated in vacuo to afford the title compound (82 mg, 75%) as a yellow solid.

¹H NMR (300 MHz, DMSO-d₆) δ 8.77 (s, 1H), 8.09 (d, J=8.6 Hz, 1H), 8.02 (br s, 1H), 7.84 (d, J=8.6 Hz, 1H), 7.18 (s, 1H), 5.00-4.19 (m, 4H), 3.95 (s, 2H), 3.80 (t, J=4.5 Hz, 4H), 2.03 (s, 3H). HPLC (Method A): RT 2.69 min (purity 91%). MS (ES+): 343.2 (ES−): 341.2.

### Example 27

### N′-{[2-(1H-Imidazol-1-yl)-4-morpholin-4-ylpyrido[3,2-d]pyrimidin-6-yl]methyl}-N,N-dimethylethane-1,2-diamine

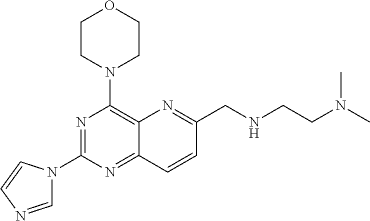

Methanesulfonyl chloride (44 mg; 0.38 mmol; 1.2 eq.) was added to a solution of Example 20 (100 mg; 0.32 mmol; 1 eq.) and DIEA (276 μl; 1.6 mmol; 5 eq.) in DCM (6 mL) and the resulting mixture was stirred at room temperature for 2 hours. 2-Dimethylaminoethylamine (105 μl; 0.96 mmol; 3 eq.) was added and the reaction mixture was stirred at room temperature for 16 hours, then concentrated in vacuo. The residue was purified by mass triggered preparative HPLC (increasing amount of ACN in water as eluent) to afford the title compound as a white solid.

¹H NMR (300 MHz, DMSO-d₆) δ 8.61 (s, 1H), 8.07 (d, J=8.1 Hz, 1H), 7.97 (s, 1H), 7.83 (d, J=8.2 Hz, 1H), 7.09 (s, 1H), 4.85-4.27 (m, 4H), 3.94 (s, 2H), 3.83-3.78 (br s, 4H), 2.61 (t, J=6.3 Hz, 2H), 2.33 (t, J=6.2 Hz, 2H), 2.11 (s, 6H). HPLC (Method A): RT 1.08 min (purity 99%). MS (ES+): 383.3.

### Example 28

### [2-(1H-Imidazol-1-yl)-4,8-dimorpholin-4-ylpyrido[3,2-d]pyrimidin-6-yl]methanol

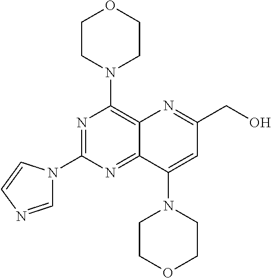

Lithium borohydride (14 mg; 0.65 mmol; 5 eq) was added to a suspension of Example 15 (56 mg; 0.13 mmol; 1 eq.) in THF (2 mL) and EtOH (2 mL) and the resulting mixture was stirred at room temperature for 16 hours then diluted with water. Extraction with DCM (3×), drying over magnesium sulfate and concentration in vacuo gave a yellow solid. Purification by column chromatography (DCM/MeOH, 94/6) followed by trituration in Et₂O afforded the title compound as an off-white solid.

¹H NMR (300 MHz, DMSO-d₆) δ 8.55 (t, J=1.0 Hz, 1H), 7.88 (t, J=1.3 Hz, 1H), 7.17 (s, 1H), 7.11-7.09 (m, 1H), 5.51 (t, J=5.9 Hz, 1H), 4.56 (d, J=5.9 Hz, 2H), 4.48 (br s, 4H), 3.87 (br t, J=4.3 Hz, 4H), 3.77 (br t, J=4.6 Hz, 4H), 3.56 (br t, J=4.5 Hz, 4H). HPLC (Method A): RT 1.33 min (purity 95%). MS (ES+): 398.2

### Example 29

### N-[2-(Dimethylamino)ethyl]-8-{[2-(dimethylamino)ethyl]amino}-2-(1H-imidazol-1-yl)-4-morpholin-4-ylpyrido[3,2-d]pyrimidine-6-carboxamide

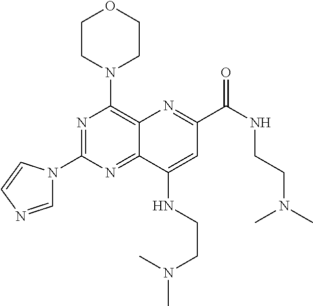

A mixture of Example 3 (200 mg; 0.53 mmol; 1 eq.) and 2-dimethylaminoethylamine (294 μl; 2.67 mmol; 5 eq.) in Dioxane (3 mL) was stirred at 150° C. for 15 minutes (microwave heating). 2-Dimethylaminoethylamine (294 μl; 2.67 mmol; 5 eq.) was added and the reaction mixture was stirred at 150° C. for 25 min then evaporated to dryness. The residue was taken up in DCM and filtered through a SPE-NH₂ column. After concentration in vacuo, the residue was crystallized from Et₂O to afford the title compound as a white solid.

¹H NMR (300 MHz, DMSO-d₆) δ 8.78 (t, J=1.0 Hz, 1H), 8.13 (t, J=1.3 Hz, 1H), 8.10 (t, J=5.3 Hz, 1H), 7.48 (t, J=5.8 Hz, 1H), 7.28 (s, 1H), 7.10 (t, J=1.2 Hz, 1H), 4.68-4.30 (m, 4H), 3.81 (t, J=4.5 Hz, 4H), 3.47-3.36 (m, 4H), 2.54 (t, J=6.5 Hz, 2H), 2.43 (t, J=6.2 Hz, 2H), 2.24 (s, 6H), 2.21 (s, 6H). HPLC (Method A): RT 1.31 min (purity 95%). MS (ES+): 483.3.

### Example 30

### Methyl 8-(dimethylamino)-2-(1H-imidazol-1-yl)-4-morpholin-4-ylpyrido[3,2-d]pyrimidine-6-carboxylate

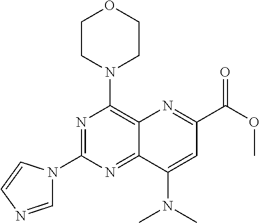

A mixture of Example 3 (100 mg; 0.27 mmol; 1 eq.) and dimethylamine (2M in THF; 4 mL; 8 mmol; 30 eq.) was stirred at 80° C. in a sealed tube for 16 hours and then evaporated to dryness. The residue was taken up in DCM, washed with sat. aq. NaHCO₃, dried over sodium sulphate and concentrated in vacuo. Recrystallization from DCM/n-pentane afforded the title compound as an off-white solid.

¹H NMR (300 MHz, DMSO-d₆) δ 8.58 (br s, 1H), 7.92 (t, J=1.2 Hz, 1H), 7.36 (s, 1H), 7.11 (br s, 1H), 4.54 (br s, 4H), 3.88 (s, 3H), 3.79 (br t, J=4.7 Hz, 4H), 3.33 (s, 6H). HPLC (Method A): RT 2.49 min (purity 99%). MS (ES+): m/z=384.2.

### Example 31

### 2-(1H-Imidazol-1-yl)-8-[(2-methoxyethyl)(methyl)amino]-4-morpholin-4-ylpyrido[3,2-d]pyrimidine-6-carboxylate

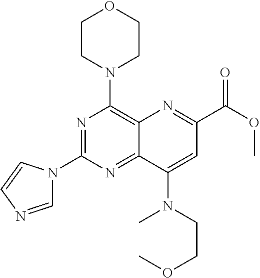

A suspension of Example 3 (100 mg; 0.27 mmol; 1 eq.) in N-(2-methoxyethyl)methylamine (0.5 mL) was stirred at 120° C. for 4 hours. The reaction mixture was diluted with water, acidified to pH 5 with AcOH and extracted with DCM (3×). The combined organic phase was washed with brine, dried over magnesium sulfate and concentrated in vacuo. The residue was taken up in MeOH (20 mL), 2 drops of conc. H₂SO₄ were added and the solution stirred at reflux for 3 hours. The solution was concentrated in vacuo to ca. 5 mL and diluted with DCM. The solution was washed with sat. aq. NaHCO₃ then brine, dried over magnesium sulfate and evaporated to dryness. Purification by column chromatography (DCM/MeOH, 97/3) followed by crystallization from Et₂O afforded the title compound as a pale yellow solid.

¹H NMR (300 MHz, DMSO-d₆) δ 8.57 (t, J=1.0 Hz, 1H), 7.91 (t, J=1.3 Hz, 1H), 7.39 (s, 1H), 7.12-7.10 (m, 1H), 4.50 (br s, 4H), 4.20 (t, J=6.1 Hz, 2H), 3.89 (s, 3H), 3.79 (br t, J=4.7 Hz, 4H), 3.71 (t, J=6.1 Hz, 2H), 3.26 (s, 3H), 3.18 (s, 3H). HPLC (Method A): RT 2.08 min (purity 96%). MS (ES+): 428.3

### Example 32

### Methyl 2-(1H-imidazol-1-yl)-8-(4-methylpiperazin-1-yl)-4-morpholin-4-ylpyrido[3,2-d]pyrimidine-6-carboxylate

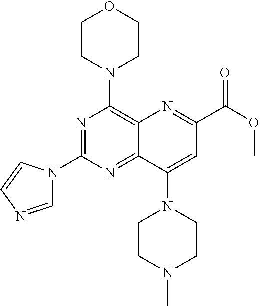

A mixture of Example 3 (200 mg; 0.53 mmol; 1 eq.) and 1-methylpiperazine (0.30 mL; 2.67 mmol; 5 eq.) in Dioxane (2 mL) was stirred at 150° C. for 15 minutes (microwave heating) then evaporated to dryness. The residue was partitioned between water and DCM and the two phases separated. The aqueous layer was extracted twice with DCM, the combined organic phase was dried over sodium sulfate and concentrated in vacuo. Recrystallization from DCM/Et₂O/n-pentane followed by recrystallization from ethyl acetate/n-pentane afforded the title compound as a pale yellow solid.

¹H NMR (300 MHz, DMSO-d₆) δ 8.57 (br s, 1H), 7.89 (t, J=1.2 Hz, 1H), 7.54 (s, 1H), 7.13 (br s, 1H), 4.85 (br s, 2H), 4.27 (br, s, 2H), 3.90 (s, 3H), 3.80 (br t, J=4.5 Hz, 4H), 3.64 (br s, 4H), 2.58 (br t, J=4.5 Hz, 4H), 2.27 (s, 3H). HPLC (Method A): RT 1.23 min (purity 95%). MS (ES+): 439.3.

### Example 33

### Methyl 2-(1H-imidazol-1-yl)-8-[(2-methoxyethyl)amino]-4-morpholin-4-ylpyrido[3,2-d]pyrimidine-6-carboxylate

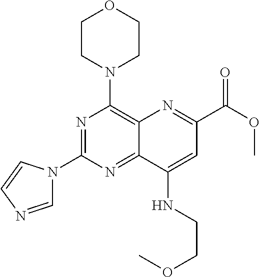

A mixture of Example 3 (120 mg; 0.32 mmol; 1 eq.) and 2-methoxyethylamine (138 μl; 1.6 mmol; 5 eq.) in Dioxane (3 mL) was stirred at 150° C. for 1 hour (microwave heating) then evaporated to dryness. The residue was partitioned between sat. aq. NH₄Cl sat and DCM and the two phases separated. The aqueous layer was extracted twice with DCM, the combined organic phase dried over sodium sulfate and concentrated in vacuo. Purification by column chromatography using increasing amount of MeOH in ethyl acetate afforded the title compound as a yellow solid

¹H NMR (300 MHz, DMSO-d₆) δ 8.84 (t, J=1.0 Hz, 1H), 8.19 (t, J=1.3 Hz, 1H), 7.62-7.55 (m, 1H), 7.31 (s, 1H), 7.10 (t, J=1.3 Hz, 1H), 5.30-4.79 (m, 2H), 4.44-4.08 (m, 2H), 3.87 (s, 3H), 3.78 (t, J=4.2 Hz, 4H), 3.61-3.52 (m, 4H), 3.29 (s, 3H). HPLC (Method A): RT 2.55 min (purity 97%). MS (ES+): 414.2 (ES−) 412.3.

### Example 34

### 2-Chloro-6-methyl-4-morpholin-4-ylpyrido[3,2-d]pyrimidine

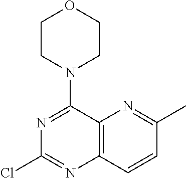

Morpholine (0.1 mL; 1.17 mmol; 1 eq.) was added slowly, at 0° C., to a solution of Intermediate 5 (250 mg; 1.17 mmol; 1 eq.) and triethylamine (0.49 mL; 3.5 mmol; 3 eq.) in ACN (10 mL) and the reaction mixture was stirred at room temperature for 48 hours. After addition of water, the solution was neutralized with NH₄Cl and extracted with DCM. The organic phase was dried over magnesium sulphate and concentrated in vacuo. Purification by column chromatography (petroleum ether/ethyl acetate, 80/20) afforded the title compound as a white solid.

¹H NMR (300 MHz, CDCl₃) δ 8.01 (d, J=8.7 Hz, 1H), 7.51 (d, J=8.7 Hz, 1H), 5.05-4.36 (m, 4H), 3.90 (br t, J=4.9 Hz, 4H), 2.68 (s, 3H). HPLC (Method A): RT 3.39 min (purity 96%). MS (ES+): 265.1.

### Example 35

### 6-Methyl-4-morpholin-4-yl-2-phenylpyrido[3,2-d]pyrimidine

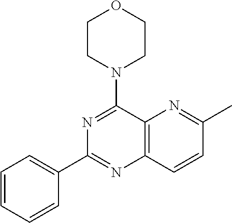

A mixture of Example 34 (200 mg; 0.76 mmol; 1 eq.), phenylboronic acid (101 mg; 0.83 mmol; 1.1 eq.), Pd(PPh₃)₄ (44 mg; 0.04 mmol; 0.05 eq.) and Cs₂CO₃ (739 mg; 2.27 mmol; 3 eq.) in dioxane (10 mL) was stirred at 150° C. for 1 hour (microwave heating). The reaction mixture was filtered through a short plug of Celite® and the solvent evaporated in vacuo until precipitation occurred. The solid was filtered to afford the title compound as a white solid.

¹H NMR (300 MHz, DMSO-d₆) δ 8.47-8.41 (m, 2H), 8.10 (d, J=8.6 Hz, 1H), 7.69 (d, J=8.6 Hz, 1H), 7.51-7.46 (m, 3H), 4.51 (br s, 4H), 3.82 (t, J=4.9 Hz, 4H), 2.63 (s, 3H). HPLC (Method A): RT 2.61 min (purity 99%). MS (ES+): 307.2.

### Example 36

### 2-(1H-Imidazol-1-yl)-6-methyl-4-morpholin-4-ylpyrido[3,2-d]pyrimidine

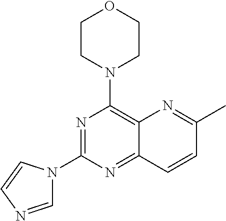

A mixture of Example 34 (300 mg; 1.13 mmol; 1 eq.), imidazole (154 mg; 2.27 mmol; 2 eq.) and potassium carbonate (783 mg; 5.67 mmol; 5 eq.) in DMA (10 mL) was stirred at 40° C. for 16 hours, then at 105° C. for 24 hours. The solvent was evaporated in vacuo and the residue taken up in EtOAc. The organic phase was washed with 5% aq. citric acid then brine, dried over magnesium sulphate and concentrated in vacuo. Purification by column chromatography (increasing amount of ethyl acetate in c-hexane) afforded the title compound as a white solid.

¹H NMR (300 MHz, DMSO-d₆) δ 8.61 (t, J=1.0 Hz, 1H), 8.01 (d, J=8.6 Hz, 1H), 7.96 (t, J=1.3 Hz, 1H), 7.69 (d, J=8.6 Hz, 1H), 7.09 (t, J=1.2 Hz, 1H), 4.56 (br s, 4H), 3.80 (br t, J=4.6 Hz, 4H), 2.62 (s, 3H). HPLC (Method A): RT 2.22 min (purity 100%). MS (ES+): 297.1.

### Example 37

### 2-(1H-Imidazol-1-yl)-4-morpholin-4-ylpyrido[3,2-d]pyrimidine

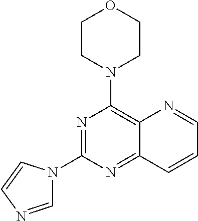

A mixture of Intermediate 10 (150 mg; 0.6 mmol; 1 eq.), imidazole (81 mg; 1.2 mmol; 2 eq.) and potassium carbonate (248 mg; 1.8 mmol; 3 eq.) in DMA (3 mL) was heated at 80° C. for 16 hours. The solvent was evaporated in vacuo and the residue diluted with water. The product was extracted with ethyl acetate, dried over magnesium sulfate and concentrated in vacuo. Crystallization from DCM/Et₂O afforded the title compound as a white solid.

¹H NMR (300 MHz, CDCl₃) δ 8.67 (dd, J=4.2, 1.7 Hz, 1H), 8.63 (d, J=1.0 Hz, 1H), 8.08 (dd, J=8.5, 1.7 Hz, 1H), 7.93 (t, J=1.4 Hz, 1H), 7.63 (dd, J=8.5, 4.2 6 Hz, 1H), 7.17 (t, J=1.2 Hz, 1H), 4.80-4.45 (m, 4H), 3.94 (br t, J=4.9 Hz, 4H). HPLC (Method A): RT 2.35 min (purity 96%). MS (ES+): 283.2.

### Example 38

### Methyl 2-(4-methoxyphenyl)-8-(methylthio)-4-morpholin-4-ylpyrido[3,2-d]pyrimidine-6-carboxylate

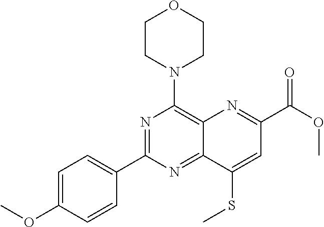

A suspension of 4-methoxyphenylboronic acid (43 mg; 0.28 mmol; 1 eq.), Example 2 (100 mg; 0.28 mmol; 1 eq.), cesium carbonate (275 mg; 0.85 mmol; 3 eq.) and Pd(PPh₃)₄ (16 mg; 0.01 mmol; 0.05 eq.) in dioxane (2.25 mL) was stirred at 90° C. for 16 h. After evaporation of the solvent, the residue was taken up in DCM and filtered through a short pad of Celite®. The solution was washed with water then brine, dried over sodium sulphate and concentrated in vacuo. The residue was triturated in MeOH, filtered and washed with MeOH then Et₂O to afford the title compound as a yellow solid.

¹H NMR (300 MHz, CDCl₃) δ 8.50 (d, J=9.0 Hz, 2H), 8.01 (s, 1H), 6.99 (d, J=9.0 Hz, 1H), 4.67 (br s, 4H), 4.01 (s, 3H), 3.93 (br t, J=6.0 Hz, 4H), 3.89 (s, 3H), 2.58 (s, 3H). HPLC (Method A): RT 4.84 min (purity 98%). MS (ES+): 427.3.

### Example 39

### 2-Imidazol-1-yl-6-(3-methyl-[1,2,4]oxadiazol-5-yl)-4-morpholin-4-yl-pyrido[3,2-d]pyrimidine

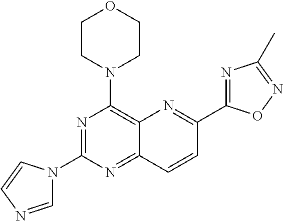

To a solution of Intermediate 11 (150 mg; 0.46 mmol) in DMF (2 mL) was added CDI (140 mg; 0.86 mmol) and the resulting mixture was stirred at room temperature for 16 hours. The reaction mixture was concentrated in vacuo and the residue taken up in toluene (5 mL). To this solution, N-hydroxy acetamidine (100 mg; 1.38 mmol) was added and the reaction mixture was refluxed for 48 hours with a Dean-Stark apparatus. After concentration in vacuo, the residue was purified by column chromatography (increasing amount of MeOH in DCM) to afford the title compound as an off white solid.

1H NMR (400 MHz, DMSO-d₆) δ 8.67 (s, 1H), 8.47 (d, J=8.7 Hz, 1H), 8.28 (d, J=8.7 Hz, 1H), 8.00 (s, 1H), 7.12 (s, 1H), 5.12 (m, 2H), 4.24 (m, 2H), 3.84 (t, J=4.7 Hz, 4H), 2.47 (s, 3H). HPLC (Method A): RT 2.74 min (purity 98%). MS (ES+): 365.1

### Example 40

### 2-Imidazol-1-yl-6-(3-methoxymethyl-[1,2,4]oxadiazol-5-yl)-4-morpholin-4-yl-pyrido[3,2-d]pyrimidine

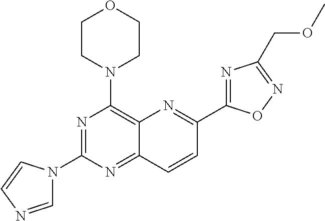

To a solution of Intermediate 11 (150 mg; 0.46 mmol) in DMF (2 mL) was added CDI (140 mg; 0.86 mmol) and the resulting mixture was stirred at room temperature for 16 hours. The reaction mixture was concentrated in vacuo and the residue taken up in toluene (5 mL). To this solution, N-hydroxy-2-methoxy-acetamidine (140 mg; 1.38 mmol) was added and the reaction mixture was refluxed for 72 hours with a dean-stark apparatus. After concentration in vacuo, the residue was purified by column chromatography (increasing amount of MeOH in DCM) to afford the title compound as a yellow solid.

¹H NMR (400 MHz, DMSO-d₆) δ 8.68 (s, 1H), 8.50 (d, J=8.8 Hz, 1H), 8.29 (d, J=8.8 Hz, 1H), 8.00 (s, 1H), 7.12 (s, 1H), 5.07 (m, 2H), 4.67 (s, 2H), 4.23 (m, 2H), 3.86-3.83 (t, J=4.5 Hz, 4H), 3.39 (s, 3H). HPLC (Method A): RT 2.70 min (purity 96%). MS (ES+): 395.2

### Example 41

### 8-Chloro-2-imidazol-1-yl-6-(3-methyl-[1,2,4]oxadiazol-5-yl)-4-morpholin-4-yl-pyrido[3,2-d]pyrimidine

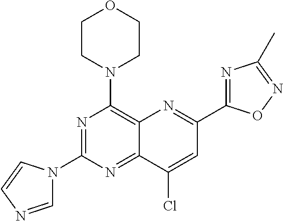

To a solution of Intermediate 12 (600 mg; 1.66 mmol) in a mixture of DCM and DMF (1:10, 10 mL), CDI (540 mg; 3.30 mmol) was added and the resulting mixture was stirred for 16 hours. The reaction mixture was concentrated in vacuo and the residue was suspended in toluene (100 mL). N-hydroxy-acetamidine (180 mg; 2.49 mmol) was added and the resulting mixture was refluxed for 48 hours with a Dean-Stark apparatus. After concentration in vacuo, the residue was purified by column chromatography (increasing amount of MeOH in DCM) to afford the title compound as a yellow solid.

1H NMR (400 MHz, DMSO-d₆) δ 8.73 (s, 1H), 8.57 (s, 1H), 7.98 (s, 1H), 7.21 (s, 1H), 5.16 (m, 2H), 4.27 (m, 2H), 3.97 (t, J=4.7 Hz, 4H), 2.55 (s, 3H). HPLC (Method A): RT 3.07 min (purity 97%). MS (ES+): 399.0.

### Example 42

### 2-Imidazol-1-yl-6-(3-methyl-[1,2,4]oxadiazol-5-yl)-4-morpholin-4-yl-8-(2-methoxy-ethyl)methylamino)-pyrido[3,2-d]pyrimidine

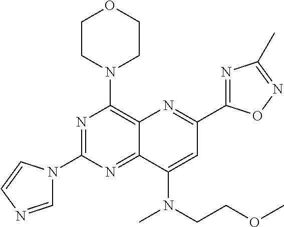

A mixture of Example 41 (30 mg; 0.075 mmol) and N-(2-ethoxyethyl)methylamine (10 mg; 0.11 mmol) in DIEA (0.5 mL) and water (0.2 mL) was stirred at 170° C. for 30 minutes (microwave heating). After concentration in vacuo, the crude was purified by preparative HPLC (from 0.1% TFA in H2O to acetonitrile). The residue was taken up in CHCl3 and washed with aq. NaHCO3, dried over magnesium sulfate and concentrated in vacuo to afford the title compound as a yellow solid.

¹H NMR (400 MHz, CDCl₃) δ 8.56 (s, 1H), 7.85 (s, 1H), 7.57 (s, 1H), 7.16 (s, 1H), 4.98 (m, 4H), 4.31 (t, J=5.5 Hz, 2H), 3.94 (m, 4H), 3.81 (t, J=5.1 Hz, 2H), 3.39 (s, 3H), 3.30 (s, 3H), 2.53 (s, 3H). HPLC (Method A): RT 3.29 min (purity 96%). MS (ES+): 452.3.

### Example 43

### 8-Dimethylamino-2-Imidazol-1-yl-6-(3-methyl-[1,2,4]oxadiazol-5-yl)-4-morpholin-4-yl-pyrido[3,2-d]pyrimidine

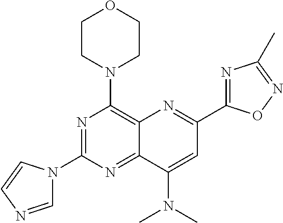

A mixture of Example 41 (75 mg; 0.19 mmol) and N,N-dimethylamine.HCl (76 mg; 0.19 mmol) in DIEA (1 mL) and water (0.5 mL) was stirred at 170° C. for 1 hour (microwave heating). After concentration in vacuo, the crude was purified by column chromatography (increasing amount of MeOH in DCM) to afford the title compound as a yellow solid.

1H NMR (400 MHz, CDCl₃) δ 8.64 (s, 1H), 7.88 (s, 1H), 7.55 (s, 1H), 7.20 (s, 1H), 4.98 (m, 4H), 3.95 (t, J=3.9 Hz, 4H), 3.44 (s, 6H), 2.52 (s, 3H). HPLC (Method A): RT 3.16 min (purity 99%). MS (ES+): 408.1.

### Example 44

### 2-Imidazol-1-yl-6-(3-methyl-[1,2,4]oxadiazol-5-yl)-8-thiomethyl-4-morpholin-4-yl-pyrido[3,2-d]pyrimidine

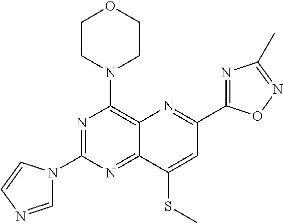

To a solution of Example 41 (600 mg; 1.5 mmol) in DMF (10 mL), sodium thiomethoxide (120 mg; 1.8 mmol) was added and the resulting mixture was stirred at room temperature for 30 minutes. After concentration in vacuo, the residue was purified by column chromatography to afford the title compound as a yellow solid.

¹H NMR (400 MHz, CDCl₃) δ 8.61 (s, 1H), 8.07 (s, 1H), 7.91 (s, 1H), 7.16 (s, 1H), 5.21 (m, 2H), 4.30 (m, 2H), 3.95 (t, J=4.88 Hz, 4H), 2.62 (s, 3H), 2.54 (s, 3H). HPLC (Method A): RT 3.16 min (purity 97%). MS (ES+): 411.0.

### Example 45

### 2-Imidazol-1-yl-6-(5-methyl-[1,3,4]oxadiazol-2-yl)-4-morpholin-4-yl-pyrido[3,2-d]pyrimidine

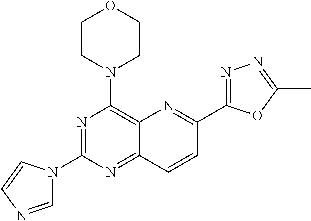

Oxalyl chloride (35 mg; 0.28 mmol) was added at 0° C. to a solution of Intermediate 11 (45 mg; 0.14 mmol) in DCM (3 mL) and DMF (2 drops) and the reaction mixture was stirred for 2 hours. After concentration in vacuo, the residue was taken up in DCM (2 mL) and added at 0° C. to a solution of acetic hydrazide (12 mg; 0.17 mmol) and DIEA (36 mg; 0.28 mmol) in DCM (2 mL). The reaction mixture was stirred at room temperature for 16 hours then concentrated in vacuo. The residue was purified by column chromatography (increasing amount of MeOH in DCM) to give 2-Imidazol-1-yl-4-morpholin-4-yl-pyrido[3,2-d]pyrimidine-6-carboxylic acid N′-acetyl-hydrazide (40 mg, 76%) as a yellow solid. The latter was suspended in phosphorous oxychloride (1 mL) and stirred at 100° C. in a sealed tube for 14 hours. After concentration in vacuo, the residue was poured into ice cold water and neutralized with aq. NaOH. The solution was extracted with ethyl acetate and the organic phase washed with brine, dried over magnesium sulfate and concentrated in vacuo. The residue was purified by column chromatography (increasing amount of MeOH in DCM) to afford the title compound as a brown solid.

¹H NMR (400 MHz, CDCl₃) δ 8.62 (s, 1H), 8.46 (d, J=8.8 Hz, 1H), 8.19 (d, J=8.8 Hz, 1H), 7.92 (s, 1H), 7.17 (s, 1H), 5.12 (m, 2H), 4.38 (m, 2H), 3.97 (t, J=4.9 Hz, 4H), 2.70 (s, 3H). HPLC (Method A): RT 2.30 min (purity 93%). MS (ES+): 365.1.

### Example 46

### 2-Imidazol-1-yl-6-(5-methyl-[1,3,4]oxadiazol-2-yl)-4-morpholin-4-yl-8-[(2-methoxy-ethyl)methylamino]-pyrido[3,2-d]pyrimidine

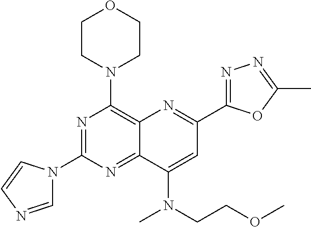

A mixture of Intermediate 13 (40 mg; 0.10 mmol) and N-(2-methoxyethyl)methylamine (13 mg, 0.15 mmol) in DIEA (1 mL) and water (0.5 mL) was stirred at 170° C. for 30 minutes (microwave heating). After concentration in vacuo, the crude was purified by preparative HPLC (from 0.1% TFA in H2O to acetonitrile). The residue was taken up in CHCl3 and washed with aq. NaHCO3, dried over magnesium sulfate and concentrated in vacuo to afford the title compound as an off-white solid.

1H NMR (400 MHz, CDCl₃) δ 8.59 (s, 1H), 7.86 (s, 1H), 7.60 (s, 1H), 7.17 (s, 1H), 4.60 (m, 4H), 4.30 (t, J=5.9 Hz, 2H), 3.94 (t, J=4.9 Hz, 4H), 3.82 (t, J=5.8 Hz, 2H), 3.39 (s, 3H), 3.29 (s, 3H), 2.68 (s, 3H). HPLC (Method A): RT 2.79 min (purity 91%). MS (ES+): 452.3.

### Example 47

### 2-Imidazol-1-yl-6-(5-methyl-[1,3,4]oxadiazol-2-yl)-4-morpholin-4-yl-8-dimethyl amino-pyrido[3,2-d]pyrimidine

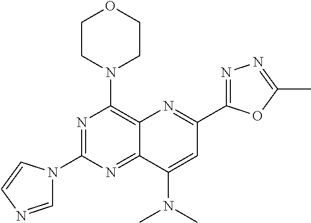

A mixture of Intermediate 13 (65 mg; 0.16 mmol) and N,N-dimethylamine.HCl (40 mg; 0.49 mmol) in DIEA (1 mL) and water (0.5 mL) was stirred at 170° C. for 1 hour (microwave heating). After concentration in vacuo, the crude was purified by column chromatography (increasing amount of MeOH in DCM) to afford the title compound as a yellow solid.

¹H NMR (400 MHz, CDCl₃) δ 8.58 (s, 1H), 7.86 (s, 1H), 7.58 (s, 1H), 7.16 (s, 1H), 4.53 (m, 4H), 3.94 (t, J=4.6 Hz, 4H), 3.43 (s, 6H), 2.67 (s, 3H). HPLC (Method A): RT 2.69 min (purity 96%). MS (ES+): 408.1.

### Example 48

### 2-Imidazol-1-yl-6-(5-methyl-[1,3,4]oxadiazol-2-yl)-4-morpholin-4-yl-8-thiomethyl-pyrido[3,2-d]pyrimidine

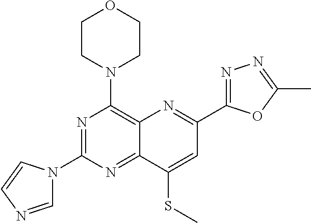

To a solution of Intermediate 13 (75 mg; 0.11 mmol) in DMF (5 mL), sodium thiomethoxide (16 mg; 0.22 mmol) was added and the resulting mixture was stirred at 90° C. for 1 hour. After concentration in vacuo, the residue was taken up in ethyl acetate, washed with water then brine, dried over sodium sulfate and concentrated in vacuo. Purification by column chromatography (increasing amount of MeOH in DCM) afforded the title compound as a yellow solid.

1H NMR (400 MHz, CDCl₃) δ 8.70 (s, 1H), 8.13 (s, 1H), 7.95 (s, 1H), 7.20 (s, 1H), 5.12 (m, 2H), 4.32 (m, 2H), 3.95 (t, J=4.7 Hz, 4H), 2.70 (s, 3H), 2.62 (s, 3H). HPLC (Method A): RT 2.68 min (purity 94%). MS (ES+): 411.0.

### Example 49

### 8-Dimethylamino-2-Imidazol-1-yl-6-methoxymethyl-4-morpholin-4-yl-pyrido[3,2-d]pyrimidine

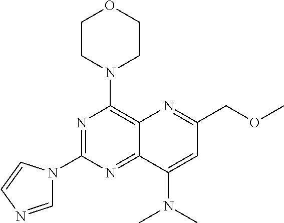

A mixture of Intermediate 15 (120 mg; 0.33 mmol) and N,N-dimethylamine.HCl (135 mg; 0.6 mmol) in DIEA (1 mL) and water (2 mL) was stirred at 170° C. for 30 minutes (microwave heating). After concentration in vacuo, the crude was purified by column chromatography (increasing amount of MeOH in DCM) to afford the title compound as an off white solid.

¹H NMR (400 MHz, CDCl3) δ 8.57 (s, 1H), 7.86 (s, 1H), 7.14 (s, 1H), 6.91 (s, 1H), 4.53 (m, 6H), 3.9022H, s), 4.21-4.34 (2H, m), 16 (t, J=4.6 Hz, 4H), 3.50 (s, 3H), 3.33 (s, 6H). HPLC (Method A): RT 2.45 min (purity 96%). MS (ES+): 370.3.

### Example 50

### 2-Imidazol-1-yl-6-methoxymethyl-8-thiomethyl-4-morpholin-4-yl-pyrido[3,2-d]pyrimidine

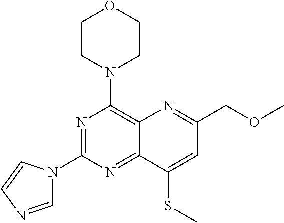

A mixture of Intermediate 15 (100 mg; 0.28 mmol) and sodium thiomethoxide (38 mg; 0.55 mmol) in DMF (3 mL) and THF (3 mL) was stirred at room temperature for 1 hour then concentrated in vacuo. The residue was purified by column chromatography (increasing amount of MeOH in DCM) to afford the title compound as an off white solid.

¹H NMR (400 MHz, DMSO-d₆) δ 8.56 (s, 1H), 7.90 (s, 1H), 7.48 (s, 1H), 7.09 (s, 1H), 5.11 (m, 2H), 4.56 (s, 2H), 4.20 (m, 2H), 3.78 (t, J=4.5 Hz, 4H), 3.39 (s, 3H), 2.51 (s, 3H). HPLC (Method A): RT 3.01 min (purity 93%). MS (ES+): 373.0.

### Example 51

### 6-(3-Methyl-1,2,4-oxadiazol-5-yl)-4-morpholin-4-yl-2-(1H-pyrazol-1-yl)pyrido[3,2-d]pyrimidine

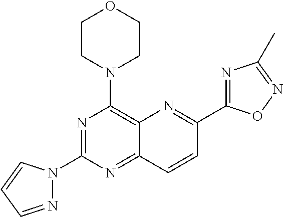

To a solution of Intermediate 17 (150 mg; 0.46 mmol) in DMF (5 mL), CDI (152 mg; 0.92 mmol) was added and the resulting mixture was stirred for 14 hours. The reaction mixture was concentrated in vacuo and the residue was suspended in toluene (40 mL). N-Hydroxy-acetamidine (51 mg; 0.69 mmol) was added and the resulting mixture was refluxed for 16 hours with a Dean-Stark apparatus. After concentration in vacuo, the residue was purified by column chromatography (increasing amount of MeOH in DCM) to afford the title compound as an off-white solid.

¹H NMR (400 MHz, CDCl₃) δ 8.60 (d, J=2.6 Hz, 1H), 8.43 (d, J=8.8 Hz, 1H), 8.35 (d, J=8.8 Hz, 1H), 7.87 (m, 1H), 6.51 (m, 1H), 5.18 (m, 2H), 4.48 (m, 2H), 3.98 (t, J=4.8 Hz, 4H), 2.54 (s, 3H). HPLC (Method A): RT 3.28 min (purity 96%). MS (ES+): 365.0.

### Example 52

### 6-[3-(Methoxymethyl)-1,2,4-oxadiazol-5-yl]-4-morpholin-4-yl-2-(1H-pyrazol-1-yl)pyrido[3,2-d]pyrimidine

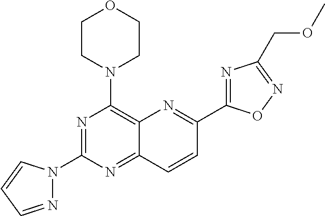

To a solution of Intermediate 17 (150 mg; 0.46 mmol) in DMF (5 mL), CDI (152 mg; 0.92 mmol) was added and the resulting mixture was stirred for 14 hours. The reaction mixture was concentrated in vacuo and the residue was suspended in toluene (40 mL). N-hydroxy-2-methoxy-acetamidine (61 mg; 0.69 mmol) was added and the resulting mixture was refluxed for 16 hours with a Dean-Stark apparatus. After concentration in vacuo, the residue was purified by column chromatography (increasing amount of MeOH in DCM) to afford the title compound as an off-white solid.

¹H NMR (400 MHz, CDCl₃) δ 8.60 (d, J=2.6 Hz, 1H), 8.50 (d, J=8.8 Hz, 1H), 8.35 (d, J=8.8 Hz, 1H), 7.87 (s, 1H), 6.51 (d, J=1.5 Hz, 1H), 5.19 (m, 2H), 4.71 (s, 2H), 4.32 (m, 2H), 3.98 (t, J=4.5 Hz, 4H), 3.56 (s, 3H). HPLC (Method A): RT 3.31 min (purity 97%). MS (ES+): 395.0.

### Example 53

### 8-[(2-Methoxy-ethyl)-methylamino]-6-(3-methyll-[1,2,4]oxadiazol-5-yl)-4-morpholin-4-yl-2-pyrazol-1-yl-pyrido[3,2-d]pyrimidine

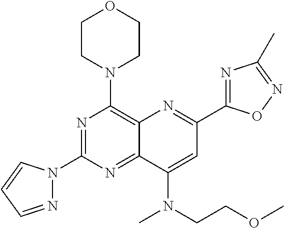

A mixture of Intermediate 19 (100 mg; 0.25 mmol) and N-(2-methoxyethyl)methylamine (45 mg, 0.50 mmol) in DI EA (1 mL) and water (0.5 mL) was stirred at 170° C. for 30 minutes (microwave heating). After concentration in vacuo, the crude was purified by column chromatography (increasing amount of MeOH in DCM) to afford the title compound as a yellow solid.

¹H NMR (400 MHz, CDCl₃) δ 8.56 (d, J=2.2 Hz, 1H), 7.82 (m, 1H), 7.56 (s, 1H), 6.48 (m, 1H), 5.00 (m, 4H), 4.37 (t, J=5.8 Hz, 2H), 3.96 (t, J=4.8 Hz, 4H), 3.84 (t, J=5.7 Hz, 2H), 3.36 (s, 3H), 3.29 (s, 3H), 2.52 (s, 3H). HPLC (Method A): RT 4.20 min (purity 96%). MS (ES+): 452.3.

### Example 54

### 8-Dimethylamino-[6-(3-methyl-[1,2,4]oxadiazol-5-yl)-4-morpholin-4-yl-2-pyrazol-1-yl-pyrido[3,2-d]pyrimidine

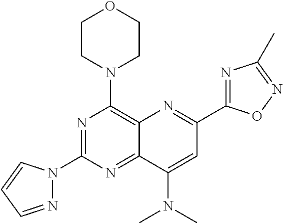

A mixture of Intermediate 19 (50 mg; 0.12 mmol) and N,N-dimethylamine.HCl (51 mg; 0.63 mmol) in DIEA (1 mL) and water (0.5 mL) was stirred at 170° C. for 30 minutes (microwave heating). After concentration in vacuo, the crude was purified by column chromatography (increasing amount of MeOH in DCM) to afford the title compound as an off-white solid.

¹H NMR (400 MHz, CDCl₃) δ 8.58 (d, J=2.6 Hz, 1H), 7.83 (s, 1H), 7.54 (s, 1H), 6.48 (t, J=1.7 Hz, 1H), 4.59 (m, 4H), 3.96 (t, J=4.9 Hz, 4H), 3.46 (s, 6H), 2.52 (s, 3H). HPLC (Method A): RT 4.11 min (purity 96%). MS (ES+): 408.1.

### Example 55

### 6-(3-Methyl-[1,2,4]oxadiazol-5-yl)-8-thiomethyl-4-morpholin-4-yl-2-pyrazol-1-yl-pyrido[3,2-d]pyrimidine

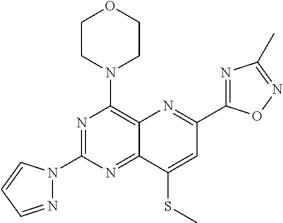

To a solution of Intermediate 19 (50 mg; 0.12 mmol) in DMF (5 mL), sodium thiomethoxide (13 mg; 0.17 mmol) was added and the resulting mixture was stirred at 90° C. for 8 hours. After concentration in vacuo, the residue was taken up in DCM, washed with water then brine, dried over sodium sulfate and concentrated in vacuo. Purification by column chromatography (increasing amount of MeOH in DCM) afforded the title compound as a yellow solid.

¹H NMR (400 MHz, CDCl₃) δ 8.68 (d, J=2.6 Hz, 1H), 8.09 (s, 1H), 7.86 (s, 1H), 6.50 (t, J=1.6 Hz, 1H), 5.20 (m, 2H), 4.33 (m, 2H), 3.96 (t, J=4.7 Hz, 4H), 2.63 (s, 3H), 2.54 (s, 3H). HPLC (Method A): RT 4.10 min (purity 98%). MS (ES+): 411.0.

### Example 56

### 2-(3-Hydroxymethyl-phenyl)-8-thiomethyl-4-morpholin-4-yl-pyrido[3,2-d]pyrimidine-6-carboxylic acid dimethylamide

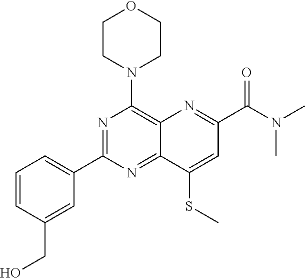

Dimethylamine (2M solution in THF; 5 mL) was added at 0° C. to a mixture of Intermediate 21 (90 mg; 0.22 mmol), triethylamine (44 mg; 0.44 mmol), EDC (68 mg; 0.44 mmol) and HOBt (58 mg; 0.44 mmol) in DCM (5 mL) and the reaction mixture was stirred at room temperature for 18 hours. After concentration in vacuo, the residue was purified by column chromatography (increasing amount of MeOH in DCM) to afford the title compound as a white solid.

¹H NMR (400 MHz, CDCl₃) δ 8.51 (s, 1H), 8.48 (m, 1H), 7.63 (s, 1H), 7.51 (m, 2H), 4.82 (s, 2H), 4.59 (m, 4H), 3.91 (t, J=4.6 Hz, 4H), 3.20 (s, 3H), 3.15 (s, 3H), 2.55 (s, 3H). HPLC (Method A): RT 3.64 min (purity 96%). MS (ES+): 440.0.

### Example 57

### 2-(1H-Indazol-4-yl)-8-thiomethyl-4-morpholin-4-yl-pyrido[3,2-d]pyrimidine-6-carboxylic acid dimethylamide

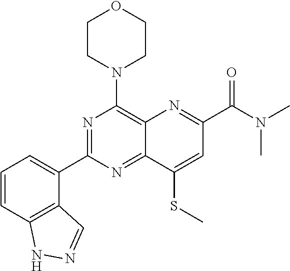

Dimethylamine (2M solution in THF; 5 mL) was added at 0° C. to a mixture of Intermediate 23 (70 mg; 0.16 mmol), triethylamine (34 mg; 0.33 mmol), EDC (52 mg; 0.33 mmol) and HOBt (44 mg; 0.33 mmol) in DCM (5 mL) and the reaction mixture was stirred at room temperature for 18 hours. After concentration in vacuo, the residue was purified by column chromatography (increasing amount of MeOH in DCM) to afford the title compound as an off-white solid.

¹H NMR (400 MHz, CDCl₃) δ 9.38 (s, 1H), 8.40 (d, J=7.2 Hz, 1H), 7.66 (m, 2H), 7.52 (m, 1H), 4.61 (m, 4H), 3.93 (t, J=4.4 Hz, 4H), 3.21 (s, 3H), 3.16 (s, 3H), 2.60 (s, 3H). HPLC (Method A): RT 3.80 min (purity 95%). MS (ES+): 450.0.

### Example 58

### 2-Imidazol-1-yl-6-methoxymethyl-4-morpholin-4-yl-8-morpholin-4-ylmethyl-pyrido[3,2-d]pyrimidine

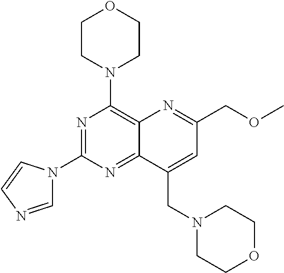

A mixture of Intermediate 15 (80 mg; 0.22 mmol), potassium(morpholin-4-yl)-methyltrifluoroborate (69 mg; 0.33 mmol), X-Phos (3 mg; 0.007 mmol), potassium carbonate (60 mg; 0.44 mmol) and palladium acetate (1 mg; 0.004 mmol) in dioxane (1 mL) and water (1 mL) was stirred at 120° C. for 2 hours (microwave heating). After concentration in vacuo, the residue was purified by column chromatography (increasing amount of MeOH in DCM) to afford the title compound as a yellow solid.

¹H NMR (400 MHz, CD₃OD) δ 9.96 (s, 1H), 8.60 (s, 1H), 8.15 (s, 1H), 7.74 (s, 1H), 5.16 (m, 2H), 4.93 (s, 2H), 4.72 (s, 2H), 4.36 (m, 2H), 3.91 (t, J=4.7 Hz, 8H), 3.54 (s, 3H), 3.47 (m, 4H). HPLC (Method A): RT 3.07 min (purity 99%). MS (ES+): 426.2.

Examples 59-72 may be prepared starting from the suitable intermediates described above and following similar procedures as for examples 1 to 58.

### Example 73

### Biological Assays

The efficacy of compounds of the invention in inhibiting the PI3K induced-lipid phosphorylation may be tested in the following binding assay. The assay combines the scintillation proximity assay technology (SPA, Amersham) with the capacity of neomycin (a polycationic antibiotic) to bind phospholipids with high affinity and specificity. The Scintillation Proximity Assay is based on the properties of weakly emitting isotopes (such as ³H, ¹²⁵I, ³³P). Coating SPA beads with neomycin allows the detection of phosphorylated lipid substrates after incubation with recombinant PI3K and radioactive ATP in the same well, by capturing the radioactive phospholipids to the SPA beads through their specific binding to neomycin. To a 96 wells MTP containing 10 μl of the test compound of Formula (I) (solubilized in 10% DMSO; to yield a concentration of 100, 25, 5.0, 1.25, 0.312, 0.078, 0.0195, 0.00488, 0.00122 and 0.0003 μM of the test compound), the following assay components are added: 1) 10 μL of lipid micelles 2) 20 mL of Kinase buffer ([³³P]γATP162 μM/300 nCi, MgCl₂ 2.5 mM, DTT 2.5 mM, Na₃VO₄ 25 μM in Hepes 40 mM, pH 7.4) and 3) 10 μL (100 ng) of Human recombinant GST-PI3K (in Hepes 40 mM, pH 7.4, ethylenglycol 4%). After incubation at room temperature for 120 minutes, with gentle agitation, the reaction is stopped by addition of 200 μL of a solution containing 250 μg of neomycin-coated PVT SPA beads, ATP 60 mM and EDTA 6.2 mM in PBS. The assay is further incubated at room temperature for 60 minutes with gentle agitation to allow binding of phospholipids to neomycin-SPA beads. After precipitation of the neomycin-coated PVT SPA beads for 5 minutes at 1500×g, radioactive Ptdlns(3)P is quantified by scintillation counting in a Wallac MicroBeta™ plate counter.

The values indicated in Table I below refer to the IC₅₀ (μM) with respect to PI3K, i.e. the amount necessary to achieve 50% inhibition of said target. Said values show a considerable inhibitory potency of pyridopyrimidine compounds with regard to PI3K.

Examples of inhibitory activities for compounds according to the invention are set out in Table I below.

TABLE I

| Example | PI3K |
|---|---|
| No | IC₅₀ (μM) |
| E-1 | 12.44 |
| E-2 | 3.53 |
| E-3 | 0.81 |
| E-4 | 1.65 |
| E-5 | 8.06 |
| E-6 | 1.83 |
| E-7 | 2.84 |
| E-8 | 1.27 |
| E-9 | 1.21 |
| E-10 | 1.27 |
| E-11 | 5.72 |
| E-12 | 0.35 |
| E-13 | 2.31 |
| E-14 | 6.86 |
| E-15 | 0.67 |
| E-16 | 0.73 |
| E-17 | 3.88 |
| E-18 | 0.43 |
| E-19 | 0.57 |
| E-20 | 3.99 |
| E-21 | 2.97 |
| E-22 | 1.10 |
| E-23 | 1.83 |
| E-24 | 3.20 |
| E-25 | 0.83 |
| E-26 | 3.76 |
| E-27 | 1.89 |
| E-28 | 2.98 |
| E-29 | 1.76 |
| E-30 | 0.42 |
| E-31 | 1.42 |
| E-32 | 1.01 |
| E-33 | 1.29 |
| E-34 | 8.61 |
| E-35 | 1.84 |
| E-36 | 0.95 |
| E-37 | 5.28 |
| E-38 | 11.44 |
| E-39 | 0.80 |
| E-40 | 0.64 |
| E-41 | 3.29 |
| E-42 | — |
| E-43 | 0.59 |
| E-44 | 3.53 |
| E-45 | 0.74 |
| E-46 | 0.42 |
| E-47 | 0.23 |
| E-48 | 0.32 |
| E-49 | 1.16 |
| E-50 | 2.31 |
| E-51 | 0.50 |
| E-52 | 0.83 |
| E-53 | 2.51 |
| E-54 | 0.48 |
| E-55 | 0.22 |
| E-56 | 0.14 |
| E-57 | 0.86 |
| E-58 | 8.45 |

1-15. (canceled)

16. A compound of Formula (I) 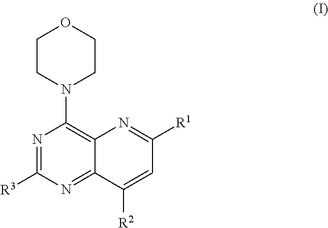
Wherein
R¹ denotes H, perfluoroalkyl, —NH₂, —NA₂, A, —NH-A, —NH—(CH₂)ₚ-A, —SO-A, SO₂-A, —COOR^{T}, —(CH₂)ₚ—OR^{T}, —(CH₂)ₚ—SR^{T}, —COA, —CO-Het, —CO—N(H)₂₋ₘ(A)ₘ; —SO—N(H)₂₋ₘ(A)ₘ, SO₂—N(H)₂₋ₘ(A)ₘ, —(CH₂)ₚ—N(H)₂₋ₘ(A)ₘ, —CO—NH—(CH₂)ₚ—N(H)₂₋ₘ(A)ₘ, —(CH₂)ₚ—NH—(CH₂)ₚ—N(H)₂₋ₘ(A)ₘ, Ar, or Het;
R² denotes H, Hal, CF₃, A, Ar, Het, SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, or —(CH₂)ₚHet, —(CH₂)ₚ—N(H)₂₋ₘ(A)ₘ;
R³ denotes Hal, Ar, OA, SA, —SOA, —SO₂A, —NH—SO₂A, CF₃, —CN, A, or —NH—SO₂Ar, or if at least one of R¹ or R² are different from H, R³ also denotes Het;
R^{T} denotes H, A, Ar, or Het;
Ar denotes a monocyclic or bicyclic, aromatic carbocyclic ring having 6 to 14 carbon atoms, which is unsubstituted or monosubstituted, disubstituted or trisubstituted by, Hal, CF₃, OCF₃, NO₂, CN, perfluoroalkyl, A, OA, NH₂, COH, CONH₂, —NHCOA, —NHSO₂A, —NHSO₂—N(H)₂₋ₘ(A)ₘ, N(H)_{1-q}A_{q}COA, N(H)_{1-q}A_{q}SO₂—N(H)₂₋ₘ(A)ₘ, —N(H)_{1-q}A_{q}CON(H)₂₋ₘ(A)ₘ, —COOA, —SO₂A, —SO₂N(H)₂₋ₘ(A)ₘ, —SO₂Het, or —(CH₂)ₚ—N(H)₂₋ₘ(A)ₘ, —(CH₂)ₚ—OR^{T}, or disubstituted or trisubstituted by OH and 1 or 2 of above described substituents;
Het denotes a monocyclic or bicyclic saturated, unsaturated or aromatic heterocyclic ring having 1, 2, 3 or 4 N, O and/or S atoms which is unsubstituted or monosubstituted, disubstituted or trisubstituted by alkyl having 1 to 8 carbon atoms, alkoxy having 1 to 8 carbon atoms, Hal, CF₃, OCF₃, NO₂, CN, perfluoroalkyl, A, OA, OH, NH₂, COH, CONH₂, —NHCOA, —NHSO₂A, —NHSO₂—N(H)₂₋ₘ(A)ₘ, N(H)_{1-q}A_{q}COA, N(H)_{1-q}A_{q}SO₂—N(H)₂₋ₘ(A)ₘ, —N(H)_{1-q}A_{q}CON(H)₂₋ₘ(A)ₘ, —COOA, —SO₂A, —SO₂N(H)₂₋ₘ(A)ₘ, —SO₂Het, —(CH₂)ₚ—N(H)₂₋ₘ(A)ₘ, or —(CH₂)ₚ—OR^{T};
m denotes 0, 1 or 2;
p denotes 0, 1, 2, 3 or 4;
q denotes 0 or 1;
A is a branched or linear alkyl having 1 to 12 C-atoms, wherein one or more, H-atoms may be replaced by Hal, Ar, Het, OR⁶, —CN, —COOalkyl or N(R⁶)₂ and wherein one or more, non-adjacent CH₂-groups, excluding the carbon atom which is linked to the rest of the molecule, may be replaced by O, NR⁶ or S and/or by —CH═CH— or —C≡C— groups, or denotes cycloalkyl or cycloalkylalkylene having 3-7 ring C atoms; and
R⁶ is H, A, —(CH₂)ₚ—N(H)₂₋ₘ(A)ₘ, —(CH₂)ₚ—OA or CH₂NH₂,
with the proviso that the following compound is excluded: 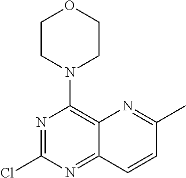
and pharmaceutically acceptable solvates, tautomers, salts and stereoisomers thereof.

17. The compound of claim 16 according to Formula (I-a): 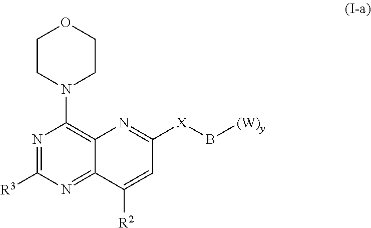
wherein
R², R³, m and p are as defined above;
X denotes CO, CS, or CH₂;
B denotes O, N, S, SO, SO₂ or a bond;
W denotes H, A, —(CH₂)ₚ—N(H)₂₋ₘ(A)ₘ, —(CH₂)ₚ—OA; or —(CH₂)ₚNH₂; and
y is 1 or 2
and pharmaceutically acceptable solvates, tautomers, salts and stereoisomers thereof.

18. The compound of claim 16 according to Formula (I-e): 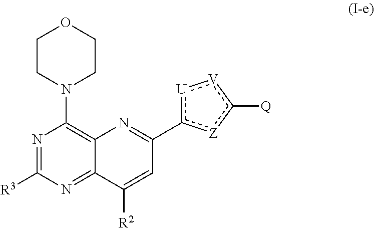
wherein R² is as defined above;
R³ is Het;
U, V and Z are independently of one another CH, O, S or N; [symbol]
is a single or a double bond; and
Q is H, Hal, CF₃, A; SA, OA, OH, —SOA, —SO₂A, —OCO-A, —N(H)₂₋ₘ(A)ₘ, —NH—(CH₂)ₚ—N(H)₂₋ₘ(A)ₘ, —NA-(CH₂)ₚ—OR^{T}, —NH—(CH₂)ₚ—OA, —(CH₂)ₚHet, —(CH₂)ₚ—OR^{T}, or —(CH₂)ₚ—NR^{T}, Wherein R^{T}, m and p are as defined in claim 16,
and pharmaceutically acceptable solvates, tautomers, salts and stereoisomers thereof.

19. The compound of Formula (I) of claim 16, wherein R³ is selected from methyl, NMe₂, NEt₂, —NH(CH₂)₃—CH₃, —O(CH₂)₂—NMe₂, SMe, OMe, CN, Cl,

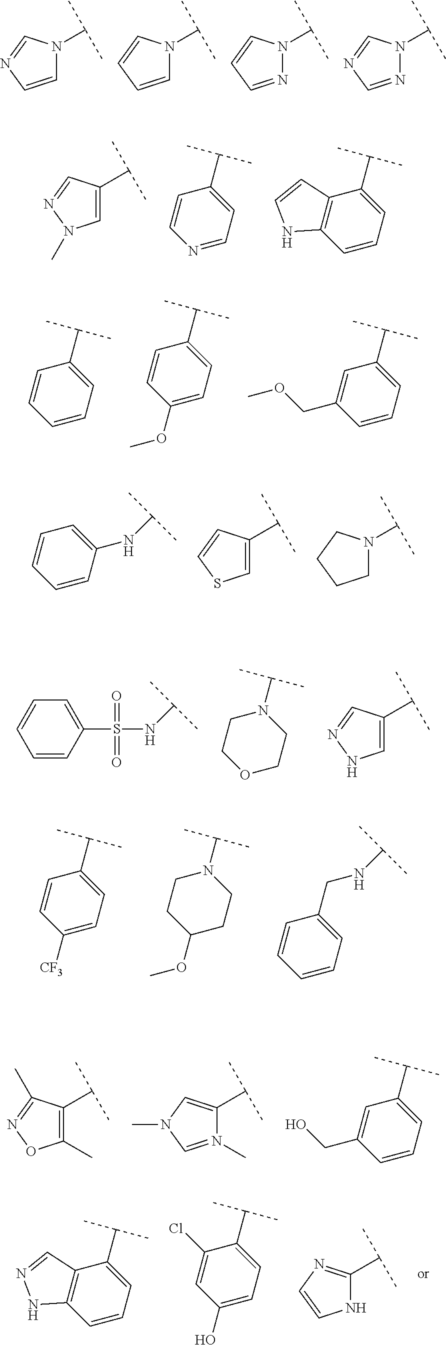

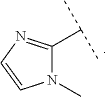

20. The compound of Formula (I) of claim 16 wherein R² is selected from H, NH—(CH₂)₂—NMe₂, —NMe₂, —NMe(CH₂)₂OMe, Cl, —SMe, —SO₂Me, Ph, —CH₂—NH—(CH₂)₂—NMe₂, —NH—(CH₂)₂—OMe, —CH₂—NMe₂,

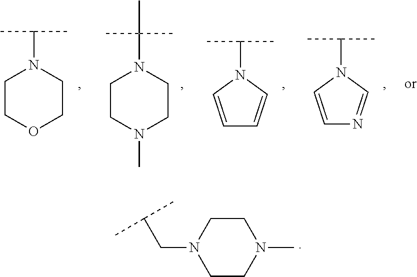

21. The compound of Formula (I) of claim 16, wherein R¹ is selected from H, —CH₃, Et, —CH₂OH, —CH₂OMe, —CH₂OCH(CH₃)₂, —CH₂NMe₂, —CH₂NHMe, —CH₂SMe, —CH₂SO₂Me, —CH₂—(NH)—(CH₂)₂—NMe₂, —CO—NH—(CH₂)₂—NMe₂, —CONMe₂, —CONHMe, —CONH₂, —CO₂Me, —CO₂Et, —CO₂H, 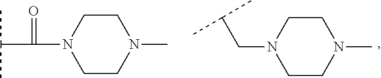
or a group selected from the following:

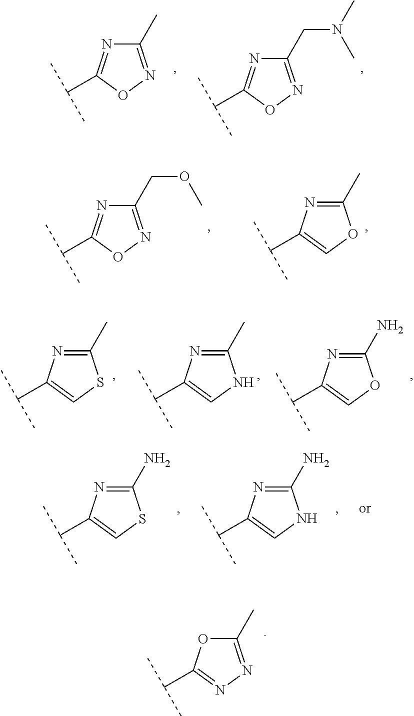

22. The compound of Formula (I) of claim 16 selected from:

| Example | Structure |
|---|---|
| E-2 | 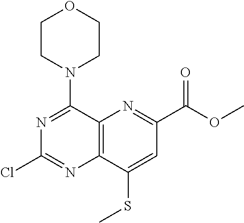 |
| E-3 | 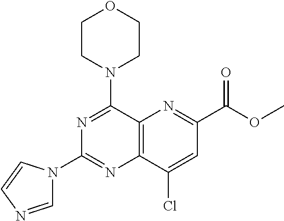 |
| E-4 | 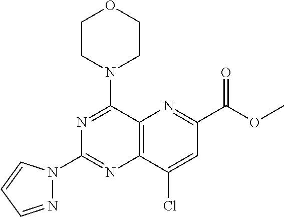 |
| E-5 | 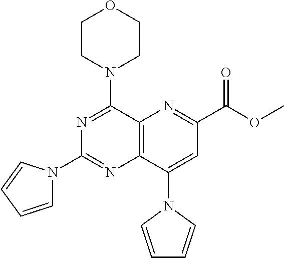 |
| E-6 | 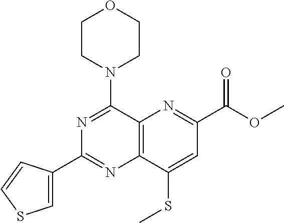 |
| E-7 | 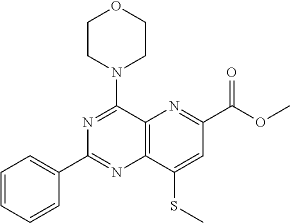 |
| E-8 | 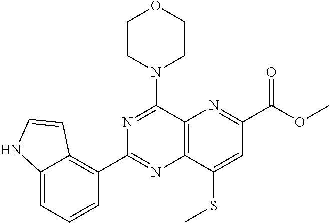 |
| E-9 | 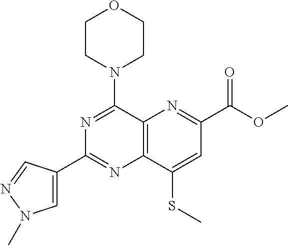 |
| E-10 | 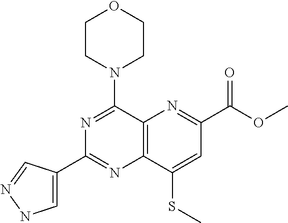 |
| E-11 | 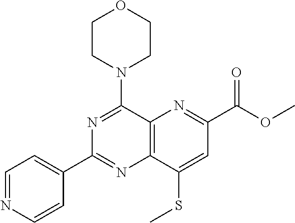 |
| E-12 | 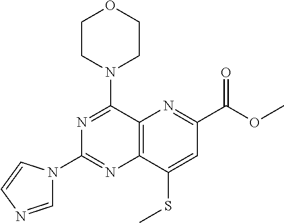 |
| E-13 | 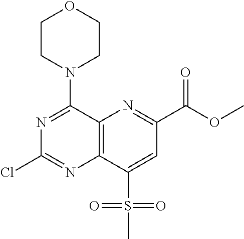 |
| E-14 | 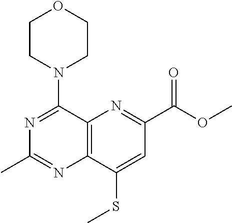 |
| E-15 | 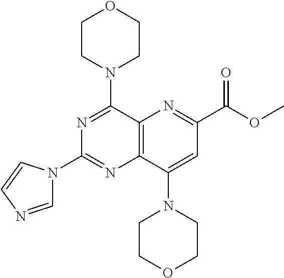 |
| E-16 | 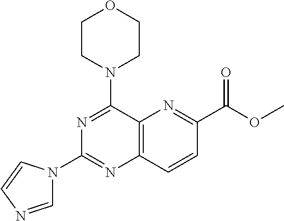 |
| E-17 | 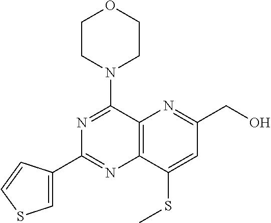 |
| E-18 | 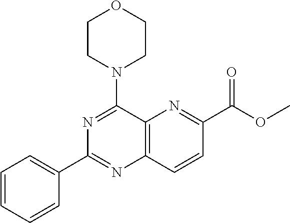 |
| E-19 | 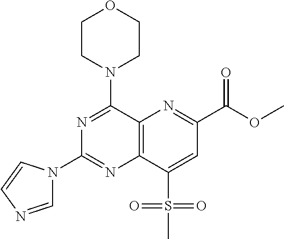 |
| E-20 | 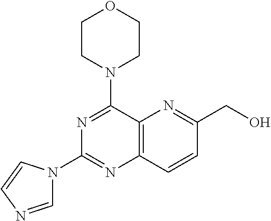 |
| E-21 | 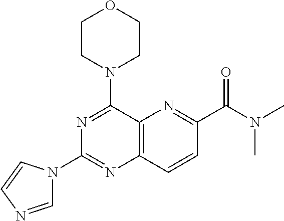 |
| E-22 | 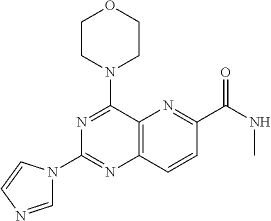 |
| E-23 | 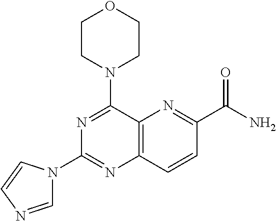 |
| E-24 | 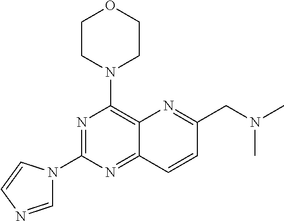 |
| E-25 | 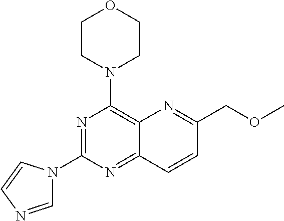 |
| E-26 | 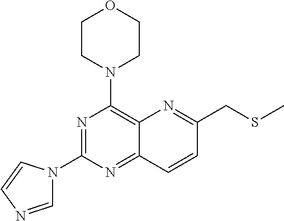 |
| E-27 | 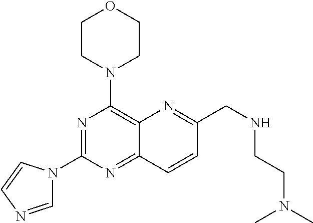 |
| E-28 | 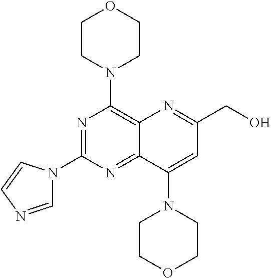 |
| E-29 | 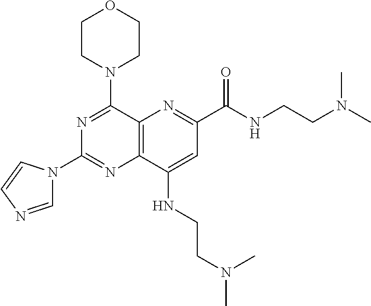 |
| E-30 | 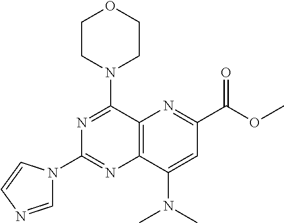 |
| E-31 | 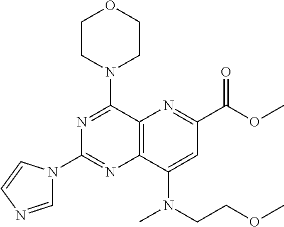 |
| E-32 | 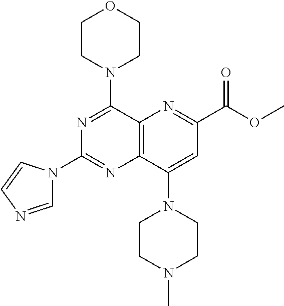 |
| E-33 | 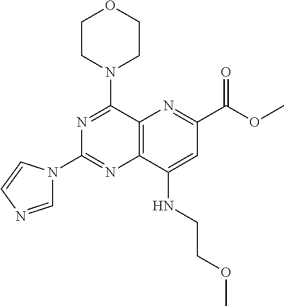 |
| E-35 | 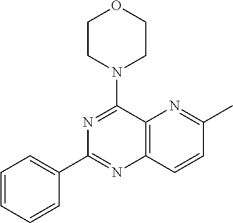 |
| E-36 | 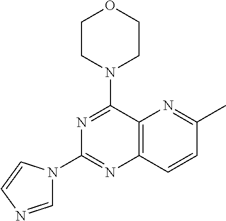 |
| E-37 | 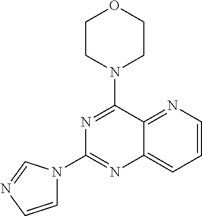 |
| E-38 | 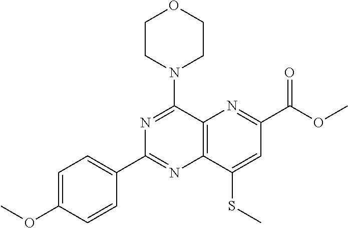 |
| E-39 | 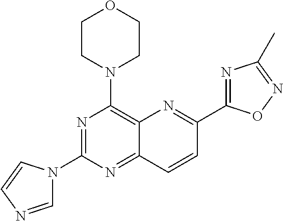 |
| E-40 | 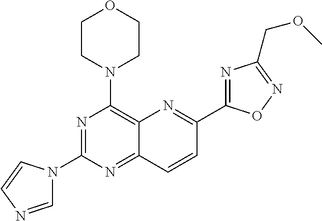 |
| E-41 | 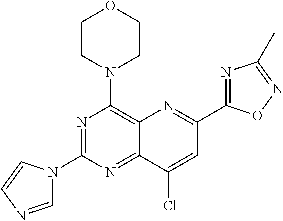 |
| E-42 | 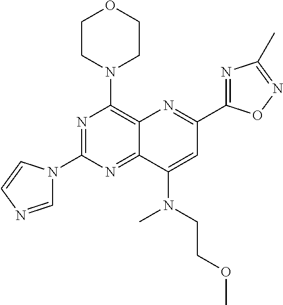 |
| E-43 | 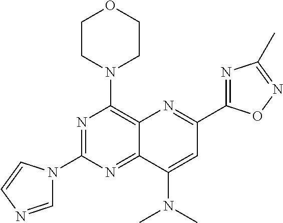 |
| E-44 | 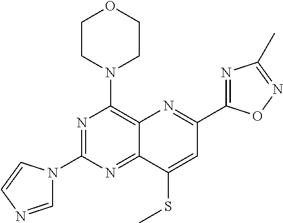 |
| E-45 | 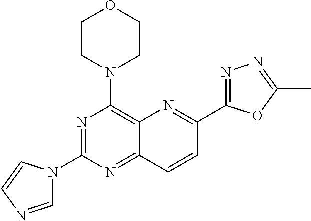 |
| E-46 | 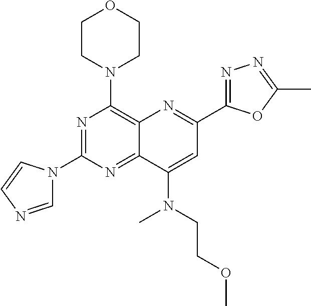 |
| E-47 | 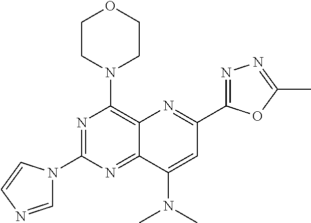 |
| E-48 | 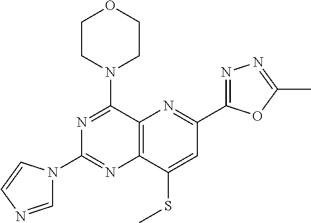 |
| E-49 | 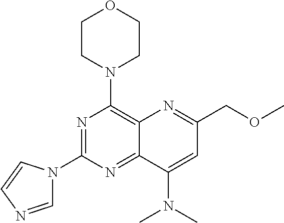 |
| E-50 | 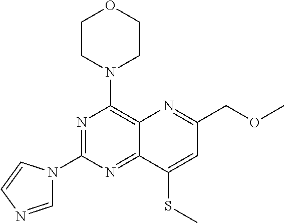 |
| E-51 | 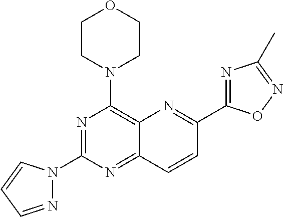 |
| E-52 | 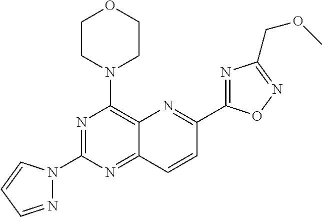 |
| E-53 | 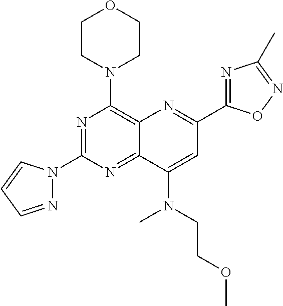 |
| E-54 | 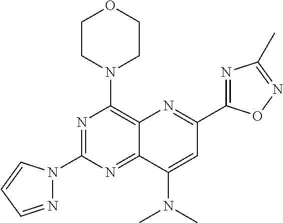 |
| E-55 | 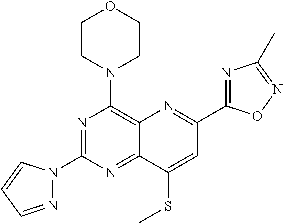 |
| E-56 | 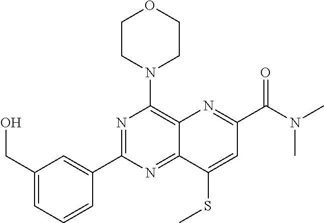 |
| E-57 | 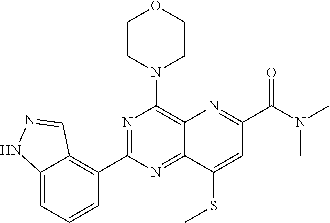 |
| E-58 | 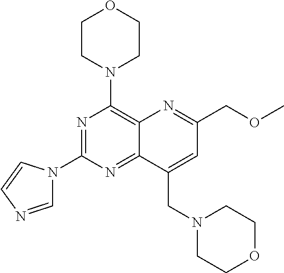 |
| E-59 | 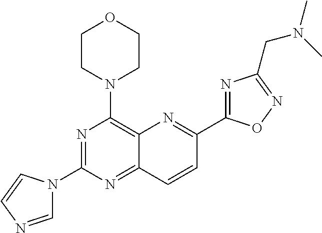 |
| E-60 | 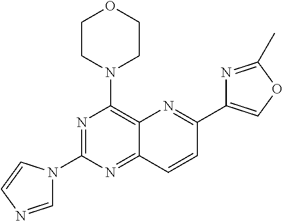 |
| E-61 | 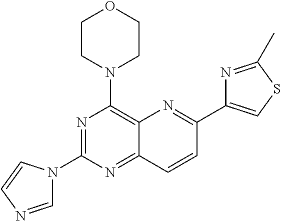 |
| E-62 | 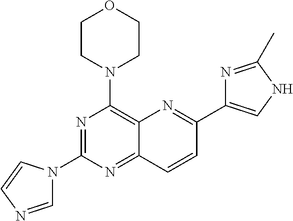 |
| E-63 | 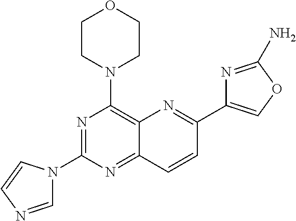 |
| E-64 | 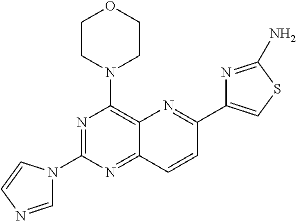 |
| E-65 | 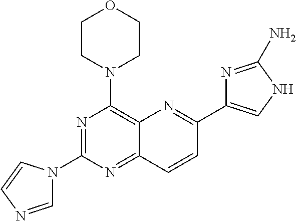 |
| E-66 | 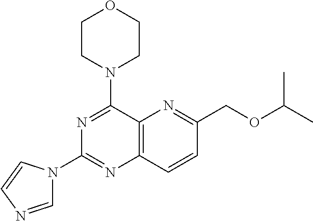 |
| E-67 | 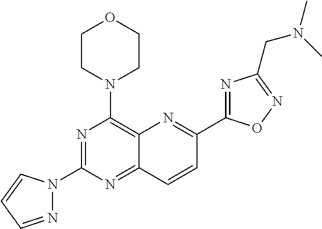 |
| E-68 | 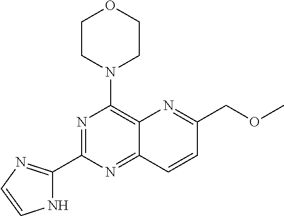 |
| E-69 | 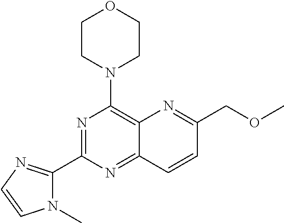 |
| E-70 | 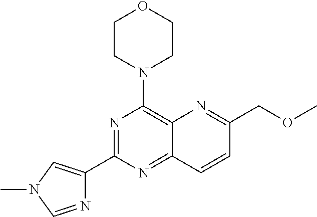 |
| E-71 | 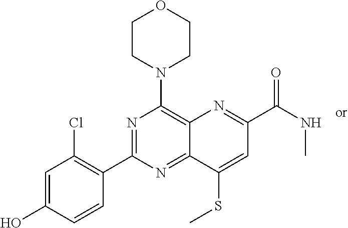 |
| E-72 | 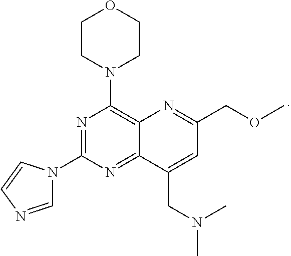 |

23. A method of treating diseases associated with Phosphoinositide 3-kinases disorders comprising the administration of a compound of claim 16 to a subject having said disorder.

24. The method of claim 23, wherein the disease is cancer, autoimmune disorder or multiple sclerosis.

25. The method of claim 23, wherein the disease is selected from the group consisting of amyotrophic lateral sclerosis (ALS), systemic lupus erythematosus, chronic rheumatoid arthritis, inflammatory bowel disease, psoriasis, autoimmune myositis, Wegener's granulomatosis, ichthyosis, bone marrow or organ transplant rejection, graft-versus-host disease, Hashimoto's thyroiditis, myasthenia gravis, uveitis, posterior uveitis, rheumatic fever, inflammatory and hyperproliferative skin diseases, atopic dermatitis, contact dermatitis, greata, keratoconjunctivitis, autoimmune hemolytic anemia, agranulocytosis, cutaneous T cell lymphoma, chronic lymphocytic leukemia, arteriosclerosis, atherosclerosis, aortitis syndrome, polyarteritis nodosa, lung cancer, carcinogenesis, metastasis of carcinoma and hypobaropathy, disease caused by histamine or leukotriene-C₄ release, autoimmune hepatitis, primary biliary cirrhosis, and Parkison disease.

26. A pharmaceutical composition comprising at least one compound of claim 16.

27. The pharmaceutical composition of claim 26, wherein said compound is combined with at least one further medicament used in the treatment of multiple sclerosis.

28. The pharmaceutical composition of claim 26, wherein said compound is combined with at least one further immunomodulating agent.

29. A process for producing a compound of Formula (I) of claim 16 comprising the transformation of the hydroxy group of compounds of Formula A into a leaving group 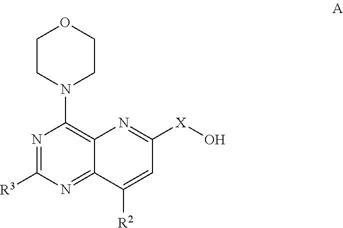
wherein R² and R³ are are as defined in claim 16 and X denotes —CH₂—.

30. A process for producing a compound of Formula (I) of claim 16, wherein R¹ is CO₂(C1-C8)alkyl or H and R² is Hal or H, comprising the reaction of the morpholine with intermediate M, 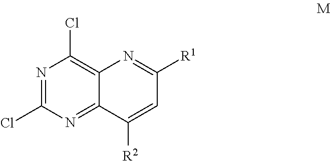
wherein R¹ is CO₂(C1-C8)alkyl or H and R² is Hal or H.
